(19) United States
(12) Patent Application Publication
(10) Pub. No.: US 2016/0371432 A1
(43) Pub. Date: Dec. 22, 2016

(54) METHODS FOR ALLELE CALLING AND PLOIDY CALLING

(71) Applicant: Natera, Inc., San Carlos, CA (US)

(72) Inventors: Matthew Rabinowitz, San Francisco, CA (US); George Gemelos, San Francisco, CA (US); Milena Banjevic, San Carlos, CA (US); Allison Ryan, Belmont, CA (US); Joshua Sweetkind-Singer, San Jose, CA (US)

(73) Assignee: Natera, Inc., San Carlos, CA (US)

(21) Appl. No.: 15/257,836

(22) Filed: Sep. 6, 2016

Related U.S. Application Data
(63) Continuation-in-part of application No. 13/846,111, filed on Mar. 18, 2013.
Application No. 13/846,111 is a continuation of application No. 13/057,350, filed on Mar. 29, 2011, now abandoned.
Application No. 13/057,350 is a continuation-in-part of application No. 12/994,260, filed on Dec. 20, 2010.
(60) Provisional application No. 61/137,851, filed on Aug. 4, 2008.
(60) Provisional application No. 61/188,343, filed on Aug. 8, 2008.
(60) Provisional application No. 61/194,854, filed on Oct. 1, 2008.
(60) Provisional application No. 61/198,690, filed on Nov. 7, 2008.
(60) Provisional application No. 61/128,961, filed on May 27, 2008.
(60) Provisional application No. 61/188,343, filed on Aug. 8, 2008.

(51) Int. Cl. G06F 19/24; G06F 19/22; G06F 19/18; C12Q 1/68

(52) U.S. Cl. CPC G06F 19/24; C12Q 1/6883; G06F 19/22; G06F 19/18; C12Q 2600/156

(57) ABSTRACT

Disclosed herein is a system and method for making allele calls, and for determining the ploidy state, in one or a small set of cells, or where a limited quantity of genetic data is available. Poorly or incorrectly measured base pairs, missing alleles and missing regions are reconstructed and the haplotypes are determined using expected similarities between the target genome and the knowledge of the genomes of genetically related individuals. In one embodiment, incomplete genetic data from an embryonic cell are reconstructed at a plurality of loci using the genetic data from both parents, and possibly one or more sperm and/or sibling embryos. In another embodiment, the chromosome copy number can be determined using the same input data. In another embodiment, these determinations are made for embryo selection during IVF, for non-invasive prenatal diagnosis, or for making phenotypic predictions.

### CROSS-REFERENCE TO RELATED APPLICATIONS

This application is a continuation-in-part of U.S. Utility application Ser. No. 13/846,111, filed on Mar. 18, 2013, which is a continuation of U.S. Utility application Ser. No. 13/057,350, filed Mar. 29, 2011, which is a national phase filing under 35 U.S.C. §371 of International Application No. PCT/US2009/052730, filed on Aug. 4, 2009. International Application No. PCT/US2009/052730 claims the benefit of U.S. Provisional Application Ser. No. 61/137,851, filed Aug. 4, 2008, U.S. Provisional Application Ser. No. 61/188,343, filed Aug. 8, 2008, U.S. Provisional Application Ser. No. 61/194,854, filed Oct. 1, 2008, and U.S. Provisional Application Ser. No. 61/198,690, filed Nov. 7, 2008. This application is also a continuation-in-part of U.S. Utility application Ser. No. 12/994,260, filed on Dec. 20, 2010, which is a national phase filing under 35 U.S.C. 371 of International Application No. PCT/US2009/045335, filed on May 27, 2009, and claims the benefit of U.S. Provisional Application Ser. No. 61/128,961, filed May 27, 2008, and U.S. Provisional Application Ser. No. 61/188,343, filed Aug. 8, 2008. The entirety of these applications are hereby incorporated herein by reference for the teachings therein.

### FIELD

The present disclosure relates generally to the field of acquiring and manipulating high fidelity genetic data for medically predictive purposes.

### BACKGROUND

In 2006, across the globe, roughly 800,000 in vitro fertilization (IVF) cycles were run. Of the roughly 150,000 cycles run in the US, about 10,000 involved pre-implantation genetic diagnosis (PGD). Current PGD techniques are unregulated, expensive and highly unreliable: error rates for screening disease-linked loci or aneuploidy are on the order of 10%, each screening test costs roughly $5,000, and a couple is typically forced to choose between testing aneuploidy, which afflicts roughly 50% of IVF embryos, or screening for disease-linked loci, for the single cell. There is a great need for an affordable technology that can reliably determine genetic data from a single cell in order to screen in parallel for aneuploidy, monogenic diseases such as Cystic Fibrosis, and susceptibility to complex disease phenotypes for which the multiple genetic markers are known through whole-genome association studies.

Most PGD today focuses on high-level chromosomal abnormalities such as aneuploidy and balanced translocations with the primary outcomes being successful implantation and a healthy baby. The other main focus of PGD is for genetic disease screening, with the primary outcome being a healthy baby not afflicted with a genetically heritable disease for which one or both parents are carriers. In both cases, the likelihood of the desired outcome is enhanced by excluding genetically suboptimal embryos from transfer and implantation in the mother.

The process of PGD during IVF currently involves extracting a single cell from the roughly eight cells of an early-stage embryo for analysis. Isolation of single cells from human embryos, while highly technical, is now routine in IVF clinics. Both polar bodies and blastomeres have been isolated with success. The most common technique is to remove single blastomeres from day 3 embryos (6 or 8 cell stage). Embryos are transferred to a special cell culture medium (standard culture medium lacking calcium and magnesium), and a hole is introduced into the zona pellucida using an acidic solution, laser, or mechanical techniques. The technician then uses a biopsy pipette to remove a single blastomere with a visible nucleus. Features of the DNA of the single (or occasionally multiple) blastomere are measured using a variety of techniques. Since only a single copy of the DNA is available from one cell, direct measurements of the DNA are highly error-prone, or noisy. There is a great need for a technique that can correct, or make more accurate, these noisy genetic measurements.

Normal humans have two sets of 23 chromosomes in every diploid cell, with one copy coming from each parent. Aneuploidy, the state of a cell with extra or missing chromosome(s), and uniparental disomy, the state of a cell with two of a given chromosome which both originate from one parent, are believed to be responsible for a large percentage of failed implantations and miscarriages, and some genetic diseases. When only certain cells in an individual are aneuploid, the individual is said to exhibit mosaicism. Detection of chromosomal abnormalities can identify individuals or embryos with conditions such as Down syndrome, Klinefelter's syndrome, and Turner syndrome, among others, in addition to increasing the chances of a successful pregnancy. Testing for chromosomal abnormalities is especially important as the age of a potential mother increases: between the ages of 35 and 40 it is estimated that between 40% and 50% of the embryos are abnormal, and above the age of 40, more than half of the embryos are like to be abnormal. The main cause of aneuploidy is nondisjunction during meiosis. Maternal nondisjunction constitutes approximately 88% of all nondisjunction of which about 65% occurs in meiosis I and 23% in meiosis II. Common types of human aneuploidy include trisomy from meiosis I nondisjunction, monosomy, and uniparental disomy. In a particular type of trisomy that arises in meiosis II nondisjunction, or M2 trisomy, an extra chromosome is identical to one of the two normal chromosomes. M2 trisomy is particularly difficult to detect. There is a great need for a better method that can detect many or all types of aneuploidy at most or all of the chromosomes efficiently and with high accuracy, including a method that can differentiate not only euploidy from aneuploidy, but also that can differentiate different types of aneuploidy from one another.

Karyotyping, the traditional method used for the prediction of aneuploidy and mosaicism is giving way to other more high-throughput, more cost effective methods such as Flow Cytometry (FC) and fluorescent in situ hybridization (FISH). Currently, the vast majority of prenatal diagnoses use FISH, which can determine large chromosomal aberrations and PCR/electrophoresis, and which can determine a handful of SNPs or other allele calls. One advantage of FISH is that it is less expensive than karyotyping, but the technique is complex and expensive enough that generally a small selection of chromosomes are tested (usually chromosomes 13, 18, 21, X, Y; also sometimes 8, 9, 15, 16, 17, 22); in addition, FISH has a low level of specificity. Roughly seventy-five percent of PGD today measures high-level chromosomal abnormalities such as aneuploidy using FISH with error rates on the order of 10-15%. There is a great demand for an aneuploidy screening method that has a higher throughput, lower cost, and greater accuracy.

The number of known disease associated genetic alleles is over 380 according to OMIM and steadily climbing. Consequently, it is becoming increasingly relevant to analyze multiple positions on the embryonic DNA, or loci, that are associated with particular phenotypes. A clear advantage of pre-implantation genetic diagnosis over prenatal diagnosis is that it avoids some of the ethical issues regarding possible choices of action once undesirable phenotypes have been detected. A need exists for a method for more extensive genotyping of embryos at the pre-implantation stage.

There are a number of advanced technologies that enable the diagnosis of genetic aberrations at one or a few loci at the single-cell level. These include interphase chromosome conversion, comparative genomic hybridization, fluorescent PCR, mini-sequencing and whole genome amplification. The reliability of the data generated by all of these techniques relies on the quality of the DNA preparation. Better methods for the preparation of single-cell DNA for amplification and PGD are therefore needed and are under study. All genotyping techniques, when used on single cells, small numbers of cells, or fragments of DNA, suffer from integrity issues, most notably allele drop out (ADO). This is exacerbated in the context of in-vitro fertilization since the efficiency of the hybridization reaction is low, and the technique must operate quickly in order to genotype the embryo within the time period of maximal embryo viability. There exists a great need for a method that alleviates the problem of a high ADO rate when measuring genetic data from one or a small number of cells, especially when time constraints exist.

### SUMMARY

Methods of embryo comparison and characterization are disclosed herein. According to aspects illustrated herein, there is provided a method for comparing embryos, the method including: obtaining one or more cells from each embryo in a set of embryos; determining one or more characteristic of each obtained cell; and estimating a likelihood that each embryo will develop as desired, based on the one or more characteristic of the one or more cells which were obtained from that embryo.

According to aspects illustrated herein, there is provided a method of characterizing an embryo for insertion into a uterus, the method including: selecting at least one characteristic; determining a first at least one characteristic of at least one cell from an embryo; using the determined first characteristic, predicting a probability of a second cell from the embryo having a second characteristic; and characterizing the embryo based on the predicted probability.

In an embodiment of the present disclosure, the method is used to determine which embryos have the best chance of developing into healthy babies if those embryos are transferred to a receptive uterus. In an embodiment of the present disclosure, the method is used to increase implantation rates, and thus possibly decreasing the number of IVF cycles necessary to achieve a successful pregnancy. In an embodiment of the present disclosure, the method provides a means to group the embryos into groups, wherein each group is defined by at least one characteristic, each group may contain zero, one or more embryos, and wherein the likelihood that each embryo in a particular group will develop as desired is estimated based on the at least one characteristic. In an embodiment of the present disclosure, the method provides a means to relatively characterizing the embryos. In this embodiment, the relative characterization may include raking the embryos based on the estimated likelihood of that embryo developing as desired. In this embodiment, once relative probabilities have been determined, embryos can be ranked, and a more informed choice can be made as to which embryos to transfer. In an embodiment, the relative characterization of embryos may include ranking the embryos based on the estimated likelihood of that embryo developing as desired. In an embodiment, the ranking may be performed to select at least one embryo to insert into a uterus. In an embodiment, the method further comprises inserting an embryo into a uterus.

In an embodiment, the present disclosure provides a method that may determine which embryos are more or less likely to result in the birth of a healthy baby, based on one or more characteristics of the embryo. This may be done by categorizing embryos into different groups, or ‘bins’, where those groups have statistically different chances of developing as desired and resulting in a successful pregnancy. The bins may then be ranked by probability, and by transferring the embryos calculated to be most likely to develop as desired, an IVF clinician can maximize the chance that an IVF patient will have a healthy baby as a result of a given IVF cycle. In an embodiment, some of the characteristics used for making decisions regarding transfer of embryos may include embryo morphology, the presence or absence of aneuploidy, and the presence or absence of one or more disease-linked genes. In an embodiment, the method may be employed to rank embryos by grouping different types of aneuploidy that correlate with higher and lower potential implantation rates. In an embodiment, the type of aneuploidy may be a characteristic used to group embryos.

In an embodiment, the present disclosure may provide a method to distinguish meiosis I/II errors from mitotic errors, and to use this knowledge to rank the embryos by the likelihood that they will implant and carry to term.

The present disclosure may employ mathematical correlations between the likelihood of an embryo to implant and carry to term and aneuploidy characteristics identified in a specific embryo. Such aneuploidy characteristics may include the parental origin of a trisomy, the identity of the aneuploid chromosome, and/or the number of aneuploid chromosomes in a cell. An embodiment may use a wide range of additional correlations to differentiate and rank embryos based on their likelihood to implant and carry to term.

The systems, methods, and techniques of the present disclosure may be used in conjunction with embryo screening in the context of IVF, or prenatal testing procedures, in the context of non-invasive prenatal diagnosis. The systems, methods, and techniques of the present disclosure may lead to increasing the probability that the embryos generated by in vitro fertilization are successfully implanted. The embodiments of the present disclosure may also be used to increase the probability that an implanted embryo is carried through the full gestation period, and result in the birth of a healthy baby. In some embodiments, the systems, methods, and techniques of the present disclosure may be employed to decrease the probability that the embryos and fetuses obtained by in vitro fertilization and are implanted and gestated are at risk for a chromosomal, congenital or other genetic disorder.

In one embodiment of the present disclosure, the disclosed method enables the reconstruction of incomplete or noisy genetic data, including the determination of the identity of individual alleles, haplotypes, sequences, insertions, deletions, repeats, and the determination of chromosome copy number on a target individual, all with high fidelity, using secondary genetic data as a source of information. While the disclosure focuses on genetic data from human subjects, and more specifically on as-yet not implanted embryos or developing fetuses, as well as related individuals, it should be noted that the methods disclosed apply to the genetic data of a range of organisms, in a range of contexts. The techniques described for cleaning genetic data are most relevant in the context of pre-implantation diagnosis during in-vitro fertilization, prenatal diagnosis in conjunction with amniocentesis, chorion villus biopsy, fetal tissue sampling, and non-invasive prenatal diagnosis, where a small quantity of fetal genetic material is isolated from maternal blood. The use of this method may facilitate diagnoses focusing on inheritable diseases, chromosome copy number predictions, increased likelihoods of defects or abnormalities, as well as making predictions of susceptibility to various disease- and non-disease phenotypes for individuals to enhance clinical and lifestyle decisions.

In an embodiment of the present disclosure, a method for determining a ploidy state of at least one chromosome in a target individual includes obtaining genetic data from the target individual and from one or more related individuals; creating a set of at least one ploidy state hypothesis for each of the chromosomes of the target individual; using one or more expert techniques to determine a statistical probability for each ploidy state hypothesis in the set, for each expert technique used, given the obtained genetic data; combining, for each ploidy state hypothesis, the statistical probabilities as determined by the one or more expert techniques; and determining the ploidy state for each of the chromosomes in the target individual based on the combined statistical probabilities of each of the ploidy state hypotheses.

In an embodiment of the present disclosure, a method for determining an allelic state in a set of alleles, in a target individual, and from one or both parents of the target individual, and optionally from one or more related individuals includes obtaining genetic data from the target individual, and from the one or both parents, and from any related individuals; creating a set of at least one allelic hypothesis for the target individual, and for the one or both parents, and optionally for the one or more related individuals, where the hypotheses describe possible allelic states in the set of alleles; determining a statistical probability for each allelic hypothesis in the set of hypotheses given the obtained genetic data; and determining the allelic state for each of the alleles in the set of alleles for the target individual, and for the one or both parents, and optionally for the one or more related individuals, based on the statistical probabilities of each of the allelic hypotheses.

In an embodiment of the present disclosure, a method for determining a ploidy state of at least one chromosome in a target individual includes obtaining genetic data from the target individual, and from both parent of the target individual, and from one or more siblings of the target individual, wherein the genetic data includes data relating to at least one chromosome; determining a ploidy state of the at least one chromosome in the target individual and in the one or more siblings of the target individual by using one or more expert techniques, wherein none of the expert techniques requires phased genetic data as input; determining phased genetic data of the target individual, and of the parents of the target individual, and of the one or more siblings of the target individual, using an informatics based method, and the obtained genetic data from the target individual, and from the parents of the target individual, and from the one or more siblings of the target individual that were determined to be euploid at that chromosome; and redetermining the ploidy state of the at least one chromosome of the target individual, using one or more expert techniques, at least one of which requires phased genetic data as input, and the determined phased genetic data of the target individual, and of the parents of the target individual, and of the one or more siblings of the target individual.

In an embodiment of the present disclosure, the method makes use of knowledge of the genetic data of the target embryo, the genetic data from mother and the father such as diploid tissue samples, and possibly genetic data from one or more of the following: sperm from the father, haploid samples from the mother or blastomeres from that same or other embryos derived from the mother's and father's gametes, together with the knowledge of the mechanism of meiosis and the imperfect measurement of the target embryonic DNA, in order to reconstruct, in silico, the embryonic DNA at the location of key loci with a high degree of confidence. In one aspect of the present disclosure, genetic data derived from other related individuals, such as other embryos, brothers and sisters, grandparents or other relatives can also be used to increase the fidelity of the reconstructed embryonic DNA. In one embodiment of the present disclosure, these genetic data may be used to determine the ploidy state at one or more chromosomes on the individual. In one aspect of the present disclosure, each of the set of genetic data measured from a set of related individuals is used to increase the fidelity of the other genetic data. It is important to note that in one aspect of the present disclosure, the parental and other secondary genetic data allows the reconstruction not only of SNPs that were measured poorly, but also of insertions, deletions, repeats, and of SNPs or whole regions of DNA that were not measured at all. In another aspect of the present disclosure, the genetic data of the target individual, along with the secondary genetic data of related individuals, is used to determine the ploidy state, or copy number, at one, several, or all of the chromosomes of the individual.

In an embodiment of the present disclosure, the fetal or embryonic genomic data, with or without the use of genetic data from related individuals, can be used to detect if the cell is aneuploid, that is, where the wrong number of a chromosome is present in a cell, or if the wrong number of sexual chromosomes are present in the cell. The genetic data can also be used to detect for uniparental disomy, a condition in which two of a given chromosome are present, both of which originate from one parent. This is done by creating a set of hypotheses about the potential states of the DNA, and testing to see which hypothesis has the highest probability of being true given the measured data. Note that the use of high throughput genotyping data for screening for aneuploidy enables a single blastomere from each embryo to be used both to measure multiple disease-linked loci as well as to screen for aneuploidy.

In an embodiment of the present disclosure, the direct measurements of the amount of genetic material, amplified or unamplified, present at a plurality of loci, can be used to detect for monosomy, uniparental disomy, matched trisomy, unmatched trisomy, tetrasomy, and other aneuploidy states. One embodiment of the present disclosure takes advantage of the fact that under some conditions, the average level of amplification and measurement signal output is invariant across the chromosomes, and thus the average amount of genetic material measured at a set of neighboring loci will be proportional to the number of homologous chromosomes present, and the ploidy state may be called in a statistically significant fashion. In another embodiment, different alleles have a statistically different characteristic amplification profiles given a certain parent context and a certain ploidy state; these characteristic differences can be used to determine the ploidy state of the chromosome.

In an embodiment of the present disclosure, the ploidy state, as determined by one aspect of the present disclosure, may be used to select the appropriate input for an allele calling embodiment of the present disclosure. In another aspect of the present disclosure, the phased, reconstructed genetic data from the target individual and/or from one or more related individuals may be used as input for a ploidy calling aspect of the present disclosure. In one embodiment of the present disclosure, the output from one aspect of the present disclosure may be used as input for, or to help select appropriate input for other aspects of the present disclosure in an iterative process.

It will be recognized by a person of ordinary skill in the art, given the benefit of this disclosure, that various aspects and embodiments of this disclosure may implemented in combination or separately.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows cumulative distribution function curves for a disomic chromosome. The cumulative distribution function curves are shown for each of the parental contexts.

FIGS. 2A-2D show cumulative distribution function curves for chromosomes with varying ploidy states. FIG. 2A shows a cumulative distribution function curve for a disomic chromosome. FIG. 2B shows a cumulative distribution function curve for a nullisomic chromosome. FIG. 2C shows a cumulative distribution function curve for a monosomic chromosome. FIG. 2D shows a cumulative distribution function curve for a maternal trisomic chromosome. The relationship between cumulative distribution function curves for different parent contexts vary with the ploidy state.

FIG. 3 shows a hypothesis distribution of various ploidy states using the Whole Chromosome Mean technique disclosed herein. Monosomic, disomic and trisomic ploidy states are shown.

FIGS. 4A-4B show a distribution of the genetic data of each of the parents using the Presence of Parents technique disclosed herein. FIG. 4A shows a distribution where genetic data from each parent is present. FIG. 4B shows a distribution where genetic data from each parent is absent.

FIG. 5 shows that distributions of the genetic measurements of the father vary when genetic data is present and non-present using the Presence of Parents technique.

FIG. 6 shows a plot of a set of Single Nucleotide Polymorphisms. A normalized intensity of one channel output is plotted against the other.

FIG. 7 shows a plot of a set of Single Nucleotide Polymorphisms. A normalized intensity of one channel output is plotted against the other.

FIGS. 8A-8C show curve fits for allelic data for different ploidy hypotheses. FIG. 8A shows curve fits for allelic data for five different ploidy hypotheses using the Kernel method disclosed herein. FIG. 8B shows curve fits for allelic data for five different ploidy hypotheses using a Gaussian Fit disclosed herein. FIG. 8C shows a histogram of the measured allelic data from one context, AA|BB−BB|AA.

FIG. 9 shows a graphical representation of meiosis.

FIGS. 10A-10B show the actual hit rate versus allele call confidence for large bins. FIG. 10A shows the average actual hit rate graphed against a predicted confidence. FIG. 10B shows the relative population of the bin.

FIGS. 11A and 11B show the actual hit rate versus allele call confidence for small bins. FIG. 11A shows the average actual hit rate graphed against a predicted confidence. FIG. 11B shows the relative population of the bin.

FIGS. 12A and 12B show allele confidence plotted along a chromosome to determine a location of a crossover. FIG. 12A shows the allele call confidences for a set of alleles located along one chromosome, as averaged over a set of neighboring alleles. The sets or alleles using different methods. FIG. 12B shows a location of a crossover along the chromosome.

FIG. 13 shows an embodiment of a statistical model for the creation of mosaicism.

FIG. 14 shows embodiments of meiosis I nondisjunction, Meoisis II nondisjunction and mitotic errors.

FIGS. 15A-15B show embodiments of CDF plots for chromosomes under disomy (FIG. 15A) and unmatched trisomy (FIG. 15B).

FIG. 16 shows embodiments of a histogram of improvement in rates of normal embryo selection.

FIG. 17 shows embodiments of a probability of a blastomere being diploid based on ploidy state of biopsied cell.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The embodiments of the present disclosure are not all limited in its application to the details of construction and the arrangement of components set forth in the following description or illustrated in the drawings. Embodiments of the present disclosure are capable of being arranged in other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the present disclosure are described below with reference to illustrative embodiments. It should be understood that reference to these illustrative embodiments is not made to limit aspects of the present disclosure in any way. Instead, illustrative embodiments are used to aid in the description and understanding of various aspects of the present disclosure. Therefore, the following description is intended to be illustrative, not limiting.

The embodiments of the present disclosure may include a method for comparing embryos comprising: obtaining one or more cells from each embryo in a set of embryos; determining one or more characteristic of each obtained cell; and estimating the likelihood that each embryo will develop as desired, based on the one or more characteristic of the one or more cells which were obtained from the embryo. The embodiments of the present disclosure may include a method of characterizing an embryo for insertion into a uterus, comprising: selecting at least one characteristic; determining a first at least one characteristic of at least one cell from an embryo; using the determined first characteristic, predicting a probability of a second cell from the embryo having a second characteristic; and characterizing the embryo based on the predicted probability.

In an embodiment of the present disclosure, the method may be able to differentiate embryos that may have been shown to be aneuploid. Typically, such embryos are either discarded or else they are implanted without regard to the type of aneuploidy detected, except in the exclusion of aneuploidy that can lead to a trisomic birth. In an embodiment, the embryos may be ranked in terms of their relative likelihood to develop as desired. In an embodiment, the embryos may be selected based on the relative likelihood that the embryos may result in a normal birth. One advantage of some embodiments of this method may be to increase in the success rate of IVF cycles where this method is utilized. For example, when this embodiment was applied to an empirical data set, the embryo ranking method resulted in improvements of implantation rates of 50-80% as compared to random selection of aneuploid embryos. See Table 6.

In any of the above embodiments, more that one cell from each embryo may be used to determine the one or more characteristics of the cells in order to estimate the likelihood of the embryo developing as desired. When more than one cell is analyzed, the determining step can be performed on the group of cells from each embryo at a time. Alternatively, the determining step can be performed on single cells from each embryo in parallel or sequence for each more than one cell from each embryo.

In an embodiment, the one or more characteristic may include at least one genetic condition. In an embodiment, the one or more characteristic may include at least one physical characteristic. In an embodiment, the determination of a genetic condition may be done using an informatics based method, such as PARENTAL SUPPORT™. In an embodiment, the at least one genetic condition may include the determination of the ploidy state of the one or more cells. In this embodiment, the ploidy state may be initially determined to be euploid or aneuploid. In an embodiment, the one or more characteristic may include the determination of the subcharacteristic or type of aneuploidy found in the one or more cells. In any embodiment, the one or more characteristic may include at least one of: (i) ploidy state; (ii) any trisomies being UCA or MCA; (iii) parental origin of any aneuploidy; (iv) a physical characteristic of an embryo; (v) a presence or absence of a disease-linked gene; (vi) a count of any aneuploid chromosomes; (vii) a chromosomal identity of any aneuploid chromosomes; and (viii) any other genetic condition not listed above.

Some examples of the types of aneuploidy criteria described herein that may be used to group or rank embryos include: maternal vs. paternal trisomies, matching vs. unmatching copy errors, the number of chromosomes that are aneuploid, and/or the identity of the aneuploid chromosome(s). Empirical information indicates that embryos with maternal trisomies are less likely to develop properly, and that cells with aneuploidy at certain chromosomes are more likely to develop as desired. In addition, embryos with more chromosomes that test positive for aneuploidy are less likely to develop as desired. Theoretical explanations may account for the tendency of embryos with matching copy errors being more likely to develop as desired than those with unmatching copy errors.

In an embodiment, embryos displaying certain criteria may be excluded from possible insertion into a uterus a priori due to the detection, in at least one of the one or more cells from the embryo(s) to be excluded, of at least one of: (i) a viable trisomy; (ii) a viable uniparental disomy (UPD); (iii) an undesired disease-linked gene; and (iv) poor physical characteristics of an embryo. In this embodiment, any characteristic that would result in an embryo not developing “as desired” can be used to exclude an embryo from further grouping, ranking or further characterization. In an embodiment, any chromosomal abnormality may be used to exclude an embryo from possible insertion into a uterus.

In an embodiment of the present disclosure, the genetic state of a cell or set of cells can be determined. Copy number calling is the concept of determining the number and identity of chromosomes in a given cell, group of cells, or set of deoxyribonucleic acid (DNA). Allele calling is the concept of determining the allelic state of a given cell, group of cells, or set of DNA, at a set of alleles, including Single Nucleotide Polymorphisms (SNPs), insertions, deletions, repeats, sequences, or other base pair information. The present disclosure allows the determination of aneuploidy, as well as allele calling, from a single cell, or other small set of DNA, provided the genome of at least one or both parents are available. Some aspects of the present disclosure use the concept that within a set of related individuals there will be sets of DNA that are nearly identical, and that using the measurements of the genetic data along with a knowledge of mechanism of meiosis, it is possible to determine the genetic state of the relevant individuals, by inference, with greater accuracy that may be possible using the individual measurements alone. This is done by determining which segments of chromosomes of related individuals were involved in gamete formation and, when necessary, where crossovers may have occurred during meiosis, and therefore which segments of the genomes of related individuals are expected to be nearly identical to sections of the target genome. This may be particularly useful in the case of preimplantation genetic diagnosis, or prenatal diagnosis, wherein a limited amount of DNA is available, and where the determination of the ploidy state of a target, an embryo or fetus in these cases, has a high clinical impact.

There are many possible mathematical techniques to determine the aneuploidy state from a set of target genetic data. Some of these techniques are discussed in this disclosure, but other techniques could be used equally well. In one embodiment of the present disclosure, both qualitative and/or quantitative data may be used. In one embodiment of the present disclosure, parental data may be used to infer target genome data that may have been measured poorly, incorrectly, or not at all. In one embodiment, inferred genetic data from one or more individual can be used to increase the likelihood of the ploidy state being determined correctly. In one embodiment of the present disclosure, a plurality of techniques may be used, each of which are able to rule out certain ploidy states, or determine the relative likelihood of certain ploidy states, and the probabilities of those predictions may be combined to produce a prediction of the ploidy state with higher confidence that is possible when using one technique alone. A confidence can be computed for each chromosomal call made.

DNA measurements, whether obtained by sequencing techniques, genotyping arrays, or any other technique, contain a degree of error. The relative confidence in a given DNA measurement is affected by many factors, including the amplification method, the technology used to measure the DNA, the protocol used, the amount of DNA used, the integrity of the DNA used, the operator, and the freshness of the reagents, just to name a few. One way to increase the accuracy of the measurements is to use informatics based techniques to infer the correct genetic state of the DNA in the target based on the knowledge of the genetic state of related individuals. Since related individuals are expected to share certain aspect of their genetic state, when the genetic data from a plurality of related individuals is considered together, it is possible to identify likely errors in the measurements, and increase the accuracy of the knowledge of the genetic states of all the related individuals. In addition, a confidence may be computed for each call made.

In some aspects of the present disclosure, the target individual is an embryo, and the purpose of applying the disclosed method to the genetic data of the embryo is to allow a doctor or other agent to make an informed choice of which embryo(s) should be implanted during IVF. In another aspect of the present disclosure, the target individual is a fetus, and the purpose of applying the disclosed method to genetic data of the fetus is to allow a doctor or other agent to make an informed choice about possible clinical decisions or other actions to be taken with respect to the fetus.

### DEFINITIONS

SNP (Single Nucleotide Polymorphism) may refer to a single nucleotide that may differ between the genomes of two members of the same species. The usage of the term should not imply any limit on the frequency with which each variant occurs.

To call a SNP may refer to the act of making a decision about the true state of a particular base pair, taking into account the direct and indirect evidence.

Sequence may refer to a DNA sequence or a genetic sequence. It may refer to the primary, physical structure of the DNA molecule or strand in an individual.

Locus may refer to a particular region of interest on the DNA of an individual, which may refer to a SNP, the site of a possible insertion or deletion, or the site of some other relevant genetic variation. Disease-linked SNPs may also refer to disease-linked loci.

Allele may refer to the genes that occupy a particular locus.

To call an allele may refer to the act of determining the genetic state at a particular locus of DNA. This may involve calling a SNP, a plurality of SNPs, or determining whether or not an insertion or deletion is present at that locus, or determining the number of insertions that may be present at that locus, or determining whether some other genetic variant is present at that locus.

Correct allele call may refer to an allele call that correctly reflects the true state of the actual genetic material of an individual.

To clean genetic data may refer to the act of taking imperfect genetic data and correcting some or all of the errors or fill in missing data at one or more loci. In the context of this disclosure, this may involve using the genetic data of related individuals and the method described herein.

To increase the fidelity of allele calls may refer to the act of cleaning genetic data with respect to a set of alleles.

Imperfect genetic data may refer to genetic data with any of the following: allele dropouts, uncertain base pair measurements, incorrect base pair measurements, missing base pair measurements, uncertain measurements of insertions or deletions, uncertain measurements of chromosome segment copy numbers, spurious signals, missing measurements, other errors, or combinations thereof.

Noisy genetic data may refer to imperfect genetic data, also called incomplete genetic data.

Uncleaned genetic data may refer to genetic data as measured, that is, where no method has been used to correct for the presence of noise or errors in the raw genetic data; also called crude genetic data.

Confidence may refer to the statistical likelihood that the called SNP, allele, set of alleles, or determined number of chromosome segment copies correctly represents the real genetic state of the individual.

Ploidy calling, also “chromosome copy number calling”, or “copy number calling” (CNC), may be the act of determining the quantity and chromosomal identity of one or more chromosomes present in a cell.

Aneuploidy may refer to the state where the wrong number of chromosomes are present in a cell. In the case of a somatic human cell it may refer to the case where a cell does not contain 22 pairs of autosomal chromosomes and one pair of sex chromosomes. In the case of a human gamete, it may refer to the case where a cell does not contain one of each of the 23 chromosomes. When referring to a single chromosome, it may refer to the case where more or less than two homologous chromosomes are present.

Ploidy State may be the quantity and chromosomal identity of one or more chromosomes in a cell.

Chromosomal identity may refer to the referent chromosome number. Normal humans have 22 types of numbered autosomal chromosomes, and two types of sex chromosomes. It may also refer to the parental origin of the chromosome. It may also refer to a specific chromosome inherited from the parent. It may also refer to other identifying features of a chromosome.

The State of the Genetic Material or simply “genetic state” may refer to the identity of a set of SNPs on the DNA, it may refer to the phased haplotypes of the genetic material, and it may refer to the sequence of the DNA, including insertions, deletions, repeats and mutations. It may also refer to the ploidy state of one or more chromosomes, chromosomal segments, or set of chromosomal segments.

Allelic Data may refer to a set of genotypic data concerning a set of one or more alleles. It may refer to the phased, haplotypic data. It may refer to SNP identities, and it may refer to the sequence data of the DNA, including insertions, deletions, repeats and mutations. It may include the parental origin of each allele.

Allelic State may refer to the actual state of the genes in a set of one or more alleles. It may refer to the actual state of the genes described by the allelic data.

Matched copy error, also ‘matching chromosome aneuploidy’, or ‘MCA’ may be a state of aneuploidy where one cell contains two identical or nearly identical chromosomes. This type of aneuploidy may arise during the formation of the gametes in mitosis, and may be referred to as a mitotic non-disjunction error.

Unmatched copy error, also “Unique Chromosome Aneuploidy” or “UCA” may be a state of aneuploidy where one cell contains two chromosomes that are from the same parent, and that may be homologous but not identical. This type of aneuploidy may arise during meiosis, and may be referred to as a meiotic error.

Mosaicism may refer to a set of cells in an embryo, or other individual that are heterogeneous with respect to their ploidy state.

Homologous Chromosomes may be chromosomes that contain the same set of genes that may normally pair up during meiosis.

Identical Chromosomes may be chromosomes that contain the same set of genes, and for each gene they have the same set of alleles that are identical, or nearly identical.

Allele Drop Out or “ADO” may refer to the situation where one of the base pairs in a set of base pairs from homologous chromosomes at a given allele is not detected.

Locus Drop Out or “LDO” may refer to the situation where both base pairs in a set of base pairs from homologous chromosomes at a given allele are not detected.

Homozygous refer to having similar alleles as corresponding chromosomal loci.

Heterozygous may refer to having dissimilar alleles as corresponding chromosomal loci.

Chromosomal Region may refer to a segment of a chromosome, or a full chromosome.

Segment of a Chromosome may refer to a section of a chromosome that can range in size from one base pair to the entire chromosome.

Chromosome may refer to either a full chromosome, or also a segment or section of a chromosome.

Copies may refer to the number of copies of a chromosome segment may refer to identical copies, or it may refer to non-identical, homologous copies of a chromosome segment wherein the different copies of the chromosome segment contain a substantially similar set of loci, and where one or more of the alleles are different. Note that in some cases of aneuploidy, such as the M2 copy error, it is possible to have some copies of the given chromosome segment that are identical as well as some copies of the same chromosome segment that are not identical.

Haplotype is a combination of alleles at multiple loci that are transmitted together on the same chromosome. Haplotype may refer to as few as two loci or to an entire chromosome depending on the number of recombination events that have occurred between a given set of loci. Haplotype can also refer to a set of single nucleotide polymorphisms (SNPs) on a single chromatid that are statistically associated.

Haplotypic Data also called ‘phased data’ or ‘ordered genetic data;’ may refer to data from a single chromosome in a diploid or polyploid genome, i.e., either the segregated maternal or paternal copy of a chromosome in a diploid genome.

Phasing may refer to the act of determining the haplotypic genetic data of an individual given unordered, diploid (or polyploidy) genetic data. It may refer to the act of determining which of two genes at an allele, for a set of alleles found on one chromosome, are associated with each of the two homologous chromosomes in an individual.

Phased Data may refer to genetic data where the haplotype been determined.

Phased Allele Call Data may refer to allelic data where the allelic state, including the haplotype data, has been determined. In one embodiment, phased parental allele call data, as determined by an informatics based method, may be used as obtained genetic data in a ploidy calling aspect of the present disclosure.

Unordered Genetic Data may refer to pooled data derived from measurements on two or more chromosomes in a diploid or polyploid genome, e.g., both the maternal and paternal copies of a particular chromosome in a diploid genome.

Genetic data ‘in’, ‘of’, ‘at’, ‘from’ or ‘on’ an individual may refer to the data describing aspects of the genome of an individual. It may refer to one or a set of loci, partial or entire sequences, partial or entire chromosomes, or the entire genome.

Hypothesis may refer to a set of possible ploidy states at a given set of chromosomes, or a set of possible allelic states at a given set of loci. The set of possibilities may contain one or more elements.

Copy number hypothesis, also ‘ploidy state hypothesis,’ may refer to a hypothesis concerning how many copies of a particular chromosome are in an individual. It may also refer to a hypothesis concerning the identity of each of the chromosomes, including the parent of origin of each chromosome, and which of the parent's two chromosomes are present in the individual. It may also refer to a hypothesis concerning which chromosomes, or chromosome segments, if any, from a related individual correspond genetically to a given chromosome from an individual.

Allelic Hypothesis may refer to a possible allelic state for a given set of alleles. A set of allelic hypotheses may refer to a set of hypotheses that describe, together, all of the possible allelic states in the set of alleles. It may also refer to a hypothesis concerning which chromosomes, or chromosome segments, if any, from a related individual correspond genetically to a given chromosome from an individual.

Target Individual may refer to the individual whose genetic data is being determined. In one context, only a limited amount of DNA is available from the target individual. In one context, the target individual is an embryo or a fetus. In some embodiments, there may be more than one target individual. In some embodiments, each child, embryo, fetus or sperm that originated from a pair of parents may be considered target individuals.

Related Individual may refer to any individual who is genetically related to, and thus shares haplotype blocks with, the target individual. In one context, the related individual may be a genetic parent of the target individual, or any genetic material derived from a parent, such as a sperm, a polar body, an embryo, a fetus, or a child. It may also refer to a sibling or a grandparent.

Sibling may refer to any individual whose parents are the same as the individual in question. In some embodiments, it may refer to a born child, an embryo, or a fetus, or one or more cells originating from a born child, an embryo, or a fetus. A sibling may also refer to a haploid individual that originates from one of the parents, such as a sperm, a polar body, or any other set of haplotypic genetic matter. An individual may be considered to be a sibling of itself.

Parent may refer to the genetic mother or father of an individual. An individual will typically have two parents, a mother and a father. A parent may be considered to be an individual.

Parental context may refer to the genetic state of a given SNP, on each of the two relevant chromosomes for each of the two parents of the target.

Develop as desired, also ‘develop normally,’ may refer to a viable embryo implanting in a uterus and resulting in a pregnancy. It may also refer to the pregnancy continuing and resulting in a live birth. It may also refer to the born child being free of chromosomal abnormalities. It may also refer to the born child being free of other undesired genetic conditions such as disease-linked genes. The term ‘develop as desired’ encompasses anything that may be desired by parents or healthcare facilitators. In some cases, ‘develop as desired’ may refer to an unviable or viable embryo that is useful for medical research or other purposes.

Insertion into a uterus may refer to the process of transferring an embryo into the uterine cavity in the context of in vitro fertilization.

Clinical Decision may refer to any decision to take an action, or not to take an action, that has an outcome that affects the health or survival of an individual. In the context of IVF, a clinical decision may refer to a decision to implant or not implant one or more embryos. In the context of prenatal diagnosis, a clinical decision may refer to a decision to abort or not abort a fetus. A clinical decision may refer to a decision to conduct further testing.

Platform response may refer to the mathematical characterization of the input/output characteristics of a genetic measurement platform, and may be used as a measure of the statistically predictable measurement differences.

Informatics based method may refer to a method designed to determine the ploidy state at one or more chromosomes or the allelic state at one or more alleles by statistically inferring the most likely state, rather than by directly physically measuring the state. In one embodiment of the present disclosure, the informatics based technique may be one disclosed in this patent. In one embodiment of the present disclosure it may be PARENTAL SUPPORT™.

Expert Technique may refer to a method used to determine a genetic state. In one embodiment it may refer to a method used to determine or aid in the determination of the ploidy state of an individual. It may refer to an algorithm, a quantitative method, a qualitative method, and/or a computer based technique.

Channel Intensity may refer to the strength of the fluorescent or other signal associated with a given allele, base pair or other genetic marker that is output from a method that is used to measure genetic data. It may refer to a set of outputs. In one embodiment, it may refer to the set of outputs from a genotyping array.

Cumulative Distribution Function (CDF) curve may refer to a monotone increasing, right continuous probability distribution of a variable, where the ‘y’ coordinate of a point on the curve refers to the probability that the variable takes on a value less than or equal to the ‘x’ coordinate of the point.

### Parental Context

The parental context may refer to the genetic state of a given SNP, on each of the two relevant chromosomes for each of the two parents of the target. Note that in one embodiment, the parental context does not refer to the allelic state of the target, rather, it refers to the allelic state of the parents. The parental context for a given SNP may consist of four base pairs, two paternal and two maternal; they may be the same or different from one another. It is typically written as “m₁m₂|f₁f₂”, where m₁ and m₂ are the genetic state of the given SNP on the two maternal chromosomes, and f₁ and f₂ are the genetic state of the given SNP on the two paternal chromosomes. In some embodiments, the parental context may be written as “f₁f₂|m₁m₂”. Note that subscripts “1” and “2” refer to the genotype, at the given allele, of the first and second chromosome; also note that the choice of which chromosome is labeled “1” and which is labeled “2” is arbitrary.

Note that in this disclosure, A and B are often used to generically represent base pair identities; A or B could equally well represent C (cytosine), G (guanine), A (adenine) or T (thymine). For example, if, at a given allele, the mother's genotype was T on one chromosome, and G on the homologous chromosome, and the father's genotype at that allele is G on both of the homologous chromosomes, one may say that the target individual's allele has the parental context of AB|BB. Note that, in theory, any of the four possible alleles could occur at a given allele, and thus it is possible, for example, for the mother to have a genotype of AT, and the father to have a genotype of GC at a given allele. However, empirical data indicate that in most cases only two of the four possible base pairs are observed at a given allele. In this disclosure the discussion assumes that only two possible base pairs will be observed at a given allele, although it should be obvious to one skilled in the art how the embodiments disclosed herein could be modified to take into account the cases where this assumption does not hold.

A “parental context” may refer to a set or subset of target SNPs that have the same parental context. For example, if one were to measure 1000 alleles on a given chromosome on a target individual, then the context AA|BB could refer to the set of all alleles in the group of 1,000 alleles where the genotype of the mother of the target was homozygous, and the genotype of the father of the target is homozygous, but where the maternal genotype and the paternal genotype are dissimilar at that locus. If the parental data is not phased, and thus AB=BA, then there are nine possible parental contexts: AA|AA, AA|AB, AA|BB, AB|AA, AB|AB, AB|BB, BB|AA, BB|AB, and BB|BB. If the parental data is phased, and thus AB≠BA, then there are sixteen different possible parental contexts: AA|AA, AA|AB, AA|BA, AA|BB, AB|AA, AB|AB, AB|BA, AB|BB, BA|AA, BA|AB, BA|BA, BA|BB, BB|AA, BB|AB, BB|BA, and BB|BB. Every SNP allele on a chromosome, excluding some SNPs on the sex chromosomes, has one of these parental contexts. The set of SNPs wherein the parental context for one parent is heterozygous may be referred to as the heterozygous context.

### Hypotheses

A hypothesis may refer to a possible genetic state. It may refer to a possible ploidy state. It may refer to a possible allelic state. A set of hypotheses refers to a set of possible genetic states. In some embodiments, a set of hypotheses may be designed such that one hypothesis from the set will correspond to the actual genetic state of any given individual. In some embodiments, a set of hypotheses may be designed such that every possible genetic state may be described by at least one hypothesis from the set. In some embodiments of the present disclosure, one aspect of the method is to determine which hypothesis corresponds to the actual genetic state of the individual in question.

In another embodiment of the present disclosure, one step involves creating a hypothesis. In some embodiments it may be a copy number hypothesis. In some embodiments it may involve a hypothesis concerning which segments of a chromosome from each of the related individuals correspond genetically to which segments, if any, of the other related individuals. Creating a hypothesis may refer to the act of setting the limits of the variables such that the entire set of possible genetic states that are under consideration are encompassed by those variables.

A ‘copy number hypothesis’, also called a ‘ploidy hypothesis’, or a ‘ploidy state hypothesis’, may refer to a hypothesis concerning a possible ploidy state for a given chromosome, or section of a chromosome, in the target individual. It may also refer to the ploidy state at more than one of the chromosomes in the individual. A set of copy number hypotheses may refer to a set of hypotheses where each hypothesis corresponds to a different possible ploidy state in an individual. A normal individual contains one of each chromosome from each parent. However, due to errors in meiosis and mitosis, it is possible for an individual to have 0, 1, 2, or more of a given chromosome from each parent. In practice, it is rare to see more that two of a given chromosomes from a parent. In this disclosure, the embodiments only consider the possible hypotheses where 0, 1, or 2 copies of a given chromosome come from a parent. In some embodiments, for a given chromosome, there are nine possible hypotheses: the three possible hypothesis concerning 0, 1, or 2 chromosomes of maternal origin, multiplied by the three possible hypotheses concerning 0, 1, or 2 chromosomes of paternal origin. Let (m,f) refer to the hypothesis where m is the number of a given chromosome inherited from the mother, and f is the number of a given chromosome inherited from the father. Therefore, the nine hypotheses are (0,0), (0,1), (0,2), (1,0), (1,1), (1,2), (2,0), (2,1), and (2,2). The different hypotheses correspond to different ploidy states. For example, (1,1) refers to a normal disomic chromosome; (2,1) refers to a maternal trisomy, and (0,1) refers to a paternal monosomy. In some embodiments, the case where two chromosomes are inherited from one parent and one chromosomes is inherited from the other parent may be further differentiated into two cases: one where the two chromosomes are identical (matched copy error), and one where the two chromosomes are homologous but not identical (unmatched copy error). In these embodiments, there are sixteen possible hypotheses. It is possible to use other sets of hypotheses, and it should be obvious for one skilled in the art how to modify the disclosed method to take into account a different number of hypotheses.

In some embodiments of the present disclosure, the ploidy hypothesis may refer to a hypothesis concerning which chromosome from other related individuals correspond to a chromosome found in the target individual's genome. In some embodiments, a key to the method is the fact that related individuals can be expected to share haplotype blocks, and using measured genetic data from related individuals, along with a knowledge of which haplotype blocks match between the target individual and the related individual, it is possible to infer the correct genetic data for a target individual with higher confidence than using the target individual's genetic measurements alone. As such, in some embodiments, the ploidy hypothesis may concern not only the number of chromosomes, but also which chromosomes in related individuals are identical, or nearly identical, with one or more chromosomes in the target individual.

An allelic hypothesis, or an ‘allelic state hypothesis’ may refer to a hypothesis concerning a possible allelic state of a set of alleles. In some embodiments, a key to this method is, as described above, related individuals may share haplotype blocks, which may help the reconstruction of genetic data that was not perfectly measured. An allelic hypothesis may also refer to a hypothesis concerning which chromosomes, or chromosome segments, if any, from a related individual correspond genetically to a given chromosome from an individual. The theory of meiosis tells us that each chromosome in an individual is inherited from one of the two parents, and this is a nearly identical copy of a parental chromosome. Therefore, if the haplotypes of the parents are known, that is, the phased genotype of the parents, then the genotype of the child may be inferred as well. (The term child, here, is meant to include any individual formed from two gametes, one from the mother and one from the father.) In one embodiment of the present disclosure, the allelic hypothesis describes a possible allelic state, at a set of alleles, including the haplotypes, as well as which chromosomes from related individuals may match the chromosome(s) which contain the set of alleles.

Once the set of hypotheses have been defined, when the algorithms operate on the input genetic data, they may output a determined statistical probability for each of the hypotheses under consideration. The probabilities of the various hypotheses may be determined by mathematically calculating, for each of the various hypotheses, the value that the probability equals, as stated by one or more of the expert techniques, algorithms, and/or methods described elsewhere in this disclosure, using the relevant genetic data as input.

Once the probabilities of the different hypotheses are estimated, as determined by a plurality of techniques, they may be combined. This may entail, for each hypothesis, multiplying the probabilities as determined by each technique. The product of the probabilities of the hypotheses may be normalized. Note that one ploidy hypothesis refers to one possible ploidy state for a chromosome.

The process of ‘combining probabilities’, also called ‘combining hypotheses’, or combining the results of expert techniques, is a concept that should be familiar to one skilled in the art of linear algebra. One possible way to combine probabilities is as follows: When an expert technique is used to evaluate a set of hypotheses given a set of genetic data, the output of the method is a set of probabilities that are associated, in a one-to-one fashion, with each hypothesis in the set of hypotheses. When a set of probabilities that were determined by a first expert technique, each of which are associated with one of the hypotheses in the set, are combined with a set of probabilities that were determined by a second expert technique, each of which are associated with the same set of hypotheses, then the two sets of probabilities are multiplied. This means that, for each hypothesis in the set, the two probabilities that are associated with that hypothesis, as determined by the two expert methods, are multiplied together, and the corresponding product is the output probability. This process may be expanded to any number of expert techniques. If only one expert technique is used, then the output probabilities are the same as the input probabilities. If more than two expert techniques are used, then the relevant probabilities may be multiplied at the same time. The products may be normalized so that the probabilities of the hypotheses in the set of hypotheses sum to 100%.

In some embodiments, if the combined probabilities for a given hypothesis are greater than the combined probabilities for any of the other hypotheses, then it may be considered that that hypothesis is determined to be the most likely. In some embodiments, a hypothesis may be determined to be the most likely, and the ploidy state, or other genetic state, may be called if the normalized probability is greater than a threshold. In one embodiment, this may mean that the number and identity of the chromosomes that are associated with that hypothesis may be called as the ploidy state. In one embodiment, this may mean that the identity of the alleles that are associated with that hypothesis may be called as the allelic state. In some embodiments, the threshold may be between about 50% and about 80%. In some embodiments the threshold may be between about 80% and about 90%. In some embodiments the threshold may be between about 90% and about 95%. In some embodiments the threshold may be between about 95% and about 99%. In some embodiments the threshold may be between about 99% and about 99.9%. In some embodiments the threshold may be above about 99.9%.

### Some Embodiments

In an embodiment of the present disclosure, a method for determining a ploidy state of at least one chromosome in a target individual includes obtaining genetic data from the target individual and from one or more related individuals; creating a set of at least one ploidy state hypothesis for each of the chromosomes of the target individual; using one or more expert techniques to determine a statistical probability for each ploidy state hypothesis in the set, for each expert technique used, given the obtained genetic data; combining, for each ploidy state hypothesis, the statistical probabilities as determined by the one or more expert techniques; and determining the ploidy state for each of the chromosomes in the target individual based on the combined statistical probabilities of each of the ploidy state hypotheses.

In an embodiment, determining the ploidy state of each of the chromosomes in the target individual can be performed in the context of in vitro fertilization, and where the target individual is an embryo. In an embodiment, determining the ploidy state of each of the chromosomes in the target individual can be performed in the context of non-invasive prenatal diagnosis, and where the target individual is a fetus. Determining the ploidy state of each of the chromosomes in the target individual can be performed in the context of screening for a chromosomal condition selected from the group including, but not limited to, euploidy, nullsomy, monosomy, uniparental disomy, trisomy, matching trisomy, unmatching trisomy, tetrasomy, other aneuploidy, unbalanced translocation, deletions, insertions, mosaicism, and combinations thereof. In an embodiment, determining the ploidy state of each of the chromosomes in the target individual can be carried out for a plurality of embryos and is used to select at least one embryo for insertion into a uterus. A clinical decision is made after determining the ploidy state of each of the chromosomes in the target individual.

In some embodiments of the present disclosure, a method for determining the ploidy state of one or more chromosome in a target individual may include the following steps:

First, genetic data from the target individual and from one or more related individuals may be obtained. In an embodiment, the related individuals include both parents of the target individual. In an embodiment, the related individuals include siblings of the target individual. This genetic data for individuals may be obtained in a number of ways including, but not limited to, it may be output measurements from a genotyping platform; it may be sequence data measured on the genetic material of the individual; it may be genetic data in silico; it may be output data from an informatics method designed to clean genetic data, or it may be from other sources. The genetic material used for measurements may be amplified by a number of techniques known in the art.

The target individual's genetic data can be measured using tools and or techniques taken from a group including, but not limited to, MOLECULAR INVERSION PROBES (MIP), Genotyping Microarrays, the TAQMAN SNP Genotyping Assay, the ILLUMINA Genotyping System, other genotyping assays, fluorescent in-situ hybridization (FISH), sequencing, other high through-put genotyping platforms, and combinations thereof. The target individual's genetic data can be measured by analyzing substances taken from a group including, but not limited to, one or more diploid cells from the target individual, one or more haploid cells from the target individual, one or more blastomeres from the target individual, extra-cellular genetic material found on the target individual, extra-cellular genetic material from the target individual found in maternal blood, cells from the target individual found in maternal blood, genetic material known to have originated from the target individual, and combinations thereof. The related individual's genetic data can be measured by analyzing substances taken from a group including, but not limited to, the related individual's bulk diploid tissue, one or more diploid cells from the related individual, one or more haploid cells taken from the related individual, one or more embryos created from (a) gamete(s) from the related individual, one or more blastomeres taken from such an embryo, extra-cellular genetic material found on the related individual, genetic material known to have originated from the related individual, and combinations thereof.

Second, a set of at least one ploidy state hypothesis may be created for each of the chromosomes of the target individual. Each of the ploidy state hypotheses may refer to one possible ploidy state of the chromosome of the target individual. The set of hypotheses may include all of the possible ploidy states that the chromosome of the target individual may be expected to have.

Third, using one or more of the expert techniques discussed in this disclosure, a statistical probability may be determined for each ploidy state hypothesis in the set. In some embodiments, the expert technique may involve an algorithm operating on the obtained genetic data, and the output may be a determined statistical probability for each of the hypotheses under consideration. In an embodiment, at least one of the expert techniques uses phased parental allele call data, that is, it uses, as input, allelic data from the parents of the target individual where the haplotypes of the allelic data have been determined. In an embodiment, at least one of the expert techniques is specific to a sex chromosome. The set of determined probabilities may correspond to the set of hypotheses. In an embodiment, the statistical probability for each of the ploidy state hypotheses may involve plotting a cumulative distribution function curve for one or more parental contexts. In an embodiment, determining the statistical probability for each of the ploidy state hypotheses may involve comparing the intensities of genotyping output data, averaged over a set of alleles, to expected intenities. The mathematics underlying the various expert techniques is described elsewhere in this disclosure.

Fourth, the set of determined probabilities may then be combined. This may entail, for each hypothesis, multiplying the probabilities as determined by each technique, and it also may involve normalizing the hypotheses. In some embodiments, the probabilities may be combined under the assumption that they are independent. The set of the products of the probabilities for each hypothesis in the set of hypotheses is then output as the combined probabilities of the hypotheses.

Lastly, the ploidy state for the target individual is determined to be the ploidy state that is associated with the hypothesis whose probability is the greatest. In some cases, one hypothesis will have a normalized, combined probability greater than 90%. Each hypothesis is associated with one ploidy state, and the ploidy state associated with the hypothesis whose normalized, combined probability is greater than 90%, or some other threshold value, may be chosen as the determined ploidy state.

In another embodiment of the present disclosure, a method for determining an allelic state in a set of alleles from a target individual, from one or both of the target individual's parents, and possibly from one or more related individuals, includes obtaining genetic data from the target individual, and from the one or both parents, and from any related individuals; creating a set of at least one allelic hypothesis for the target individual, and for the one or both parents, and optionally for the one or more related individuals, where the hypotheses describe possible allelic states in the set of alleles; determining a statistical probability for each allelic hypothesis in the set of hypotheses given the obtained genetic data; and determining the allelic state for each of the alleles in the set of alleles for the target individual, and for the one or both parents, and optionally for the one or more related individuals, based on the statistical probabilities of each of the allelic hypotheses. In an embodiment, the method takes into account a possibility of DNA crossovers that may occur during meiosis. In an embodiment, the method can be performed alongside or in conjunction with a method that determines a number of copies of a given chromosome segment present in the one or more target individuals, and where both methods use a same cell, or group of cells, from the one or more target individuals as a source of genetic data.

In an embodiment, allelic state determination can be performed in the context of in vitro fertilization, and where at least one of the target individuals is an embryo. In an embodiment, allelic state determination can be performed wherein at least one of the target individuals is an embryo, and wherein determining the allelic state in the set of alleles of the one or more target individuals is performed to select at least one embryo for transfer in the context of IVF, and where the target individuals are selected from the group including, but not limited to, one or more embryos that are from the same parents, one or more sperm from the father, and combinations thereof. In an embodiment, allelic state determination can be performed in the context of non-invasive prenatal diagnosis, and where at least one of the target individuals is a fetus. In an embodiment, determining the allelic state in the set of alleles of the one or more target individuals may include a phased genotype at a set of alleles for those individuals. A clinical decision can be made after determining the allelic state in the set of alleles of the one of more target individuals.

In some embodiments of the present disclosure, a method for determining the allelic data of one or more target individuals, and one or both of the target individuals' parents, at a set of alleles, may include the following steps:

First, genetic data from the target individual(s), from one or both of the parents, and from zero or more related individuals, may be obtained. This genetic data for individuals may be obtained in a number of ways including, but not limited to, output measurements from a genotyping platform; it may be sequence data measured on the genetic material of the individual; it may be genetic data in silico; it may be output data from an informatics method designed to clean genetic data, or it may be from other sources. In an embodiment, the obtained genetic data may include single nucleotide polymorphisms measured from a genotyping array. In an embodiment, the obtained genetic data may include DNA sequence data, that is, the measured genetic sequence representing the primary structure of the DNA of the individual. The genetic material used for measurements may be amplified by a number of techniques known in the art. In one embodiment, the target individuals are all siblings. In one embodiment, one or more of the genetic measurements of the target individuals were made on single cells. In an embodiment, platform response models can be used to determine a likelihood of a true genotype given observed genetic measurements and a characteristic measurement bias of the genotyping technique.

The target individual's genetic data can be measured using tools and or techniques taken from a group including, but not limited to, MOLECULAR INVERSION PROBES (MIP), Genotyping Microarrays, the TAQMAN SNP Genotyping Assay, the ILLUMINA Genotyping System, other genotyping assays, fluorescent in-situ hybridization (FISH), sequencing, other high through-put genotyping platforms, and combinations thereof. The target individual's genetic data can be measured by analyzing substances taken from a group including, but not limited to, one or more diploid cells from the target individual, one or more haploid cells from the target individual, one or more blastomeres from the target individual, extra-cellular genetic material found on the target individual, extra-cellular genetic material from the target individual found in maternal blood, cells from the target individual found in maternal blood, genetic material known to have originated from the target individual, and combinations thereof. The related individual's genetic data can be measured by analyzing substances taken from a group including, but not limited to, the related individual's bulk diploid tissue, one or more diploid cells from the related individual, one or more haploid cells taken from the related individual, one or more embryos created from (a) gamete(s) from the related individual, one or more blastomeres taken from such an embryo, extra-cellular genetic material found on the related individual, genetic material known to have originated from the related individual, and combinations thereof.

Second, a set of a plurality of allelic hypothesis may be created for the set of alleles, for each of the individuals. Each of the allelic hypotheses may refer to a possible identity for each of the alleles over the set of alleles for that individual. In one embodiment, the identity of the alleles of a target individual may include the origin of the allele, namely, the parent from which the allele genetically originated, and the specific chromosome from which the allele genetically originated. The set of hypotheses may include all of the possible allelic states that the target individual may be expected to have within that set of alleles.

Lastly, a statistical probability for each of the allelic hypotheses may be determined given the obtained genetic data. The determination of the probability of a given hypothesis may be done using any of the algorithms described in this disclosure, specifically those in the allele calling section. The set of allelic hypotheses for an individual may include all of the possible allelic states of that individual, over the set of alleles. Those hypotheses that match more closely to the noisy measured genetic data of the target individual are more likely to be correct. The hypothesis that corresponds exactly to the actual genetic data of the target individual will most likely be determined to have a very high probability. The allelic state may be determined to be the allelic state that corresponds with the hypothesis that is determined to have the highest probability. In some embodiments, the allelic state may be determined for various subsets of the set of alleles.

### Parental Support

Some embodiments of the present disclosure may use the informatics based PARENTAL SUPPORT™ (PS) method. In some embodiments, the PARENTAL SUPPORT™ method is a collection of methods that may be used to determine the genetic data, with high accuracy, of one or a small number of cells, specifically to determine disease-related alleles, other alleles of interest, and/or the ploidy state of the cell(s).

The PARENTAL SUPPORT™ method makes use of known parental genetic data, i.e. haplotypic and/or diploid genetic data of the mother and/or the father, together with the knowledge of the mechanism of meiosis and the imperfect measurement of the target DNA, and possible of one or more related individuals, in order to reconstruct, in silico, the genotype at a plurality of alleles, and/or the ploidy state of an embryo or of any target cell(s), and the target DNA at the location of key loci with a high degree of confidence. The PARENTAL SUPPORT™ method can reconstruct not only single-nucleotide polymorphisms that were measured poorly, but also insertions and deletions, and SNPs or whole regions of DNA that were not measured at all. Furthermore, the PARENTAL SUPPORT™ method can both measure multiple disease-linked loci as well as screen for aneuploidy, from a single cell. In some embodiments, the PARENTAL SUPPORT™ method may be used to characterize one or more cells from embryos biopsied during an IVF cycle to determine the genetic condition of the one or more cells.

The PARENTAL SUPPORT™ method allows the cleaning of noisy genetic data. This may be done by inferring the correct genetic alleles in the target genome (embryo) using the genotype of related individuals (parents) as a reference. PARENTAL SUPPORT™ may be particularly relevant where only a small quantity of genetic material is available (e.g. PGD) and where direct measurements of the genotypes are inherently noisy due to the limited amounts of genetic material. The PARENTAL SUPPORT™ method is able to reconstruct highly accurate ordered diploid allele sequences on the embryo, together with copy number of chromosomes segments, even though the conventional, unordered diploid measurements may be characterized by high rates of allele dropouts, drop-ins, variable amplification biases and other errors. The method may employ both an underlying genetic model and an underlying model of measurement error. The genetic model may determine both allele probabilities at each SNP and crossover probabilities between SNPs. Allele probabilities may be modeled at each SNP based on data obtained from the parents and model crossover probabilities between SNPs based on data obtained from the HapMap database, as developed by the International HapMap Project. Given the proper underlying genetic model and measurement error model, maximum a posteriori (MAP) estimation may be used, with modifications for computationally efficiency, to estimate the correct, ordered allele values at each SNP in the embryo.

One aspect of the PARENTAL SUPPORT™ technology is a chromosome copy number calling algorithm that in some embodiments uses parental genotype contexts. To call the chromosome copy number, the algorithm may use the phenomenon of locus dropout (LDO) combined with distributions of expected embryonic genotypes. During whole genome amplification, LDO necessarily occurs. LDO rate is concordant with the copy number of the genetic material from which it is derived, i.e., fewer chromosome copies result in higher LDO, and vice versa. As such, it follows that loci with certain contexts of parental genotypes behave in a characteristic fashion in the embryo, related to the probability of allelic contributions to the embryo. For example, if both parents have homozygous BB states, then the embryo should never have AB or AA states. In this case, measurements on the A detection channel are expected to have a distribution determined by background noise and various interference signals, but no valid genotypes. Conversely, if both parents have homozygous AA states, then the embryo should never have AB or BB states, and measurements on the A channel are expected to have the maximum intensity possible given the rate of LDO in a particular whole genome amplification. When the underlying copy number state of the embryo differs from disomy, loci corresponding to the specific parental contexts behave in a predictable fashion, based on the additional allelic content that is contributed by, or is missing from, one of the parents. This allows the ploidy state at each chromosome, or chromosome segment, to be determined. The details of one embodiment of this method are described elsewhere in this disclosure.

### Copy Number Calling Using Parental Contexts

The concept of parental contexts may be useful in the context of copy number calling (also referred to as ‘ploidy determination’). When genotyped, all of the SNPs within a first parental context may be expected to statistically behave the same way when measured for a given ploidy state. In contrast, some sets of SNPs from a second parental context may be expected to statistically behave differently from those in the first parental context in certain circumstances, such as for certain ploidy states, and the difference in behavior may be characteristic of one or a set of particular ploidy states. There are many statistical techniques that could be used to analyze the measured responses at the various loci within the various parental contexts. In some embodiments of the present disclosure, statistical techniques may be used that output probabilities for each of the hypotheses. In some embodiments of the present disclosure, statistical techniques may be used that output probabilities for each of the hypotheses along with confidences in the estimated probabilities. Some techniques, when used individually, may not be adequate to determine the ploidy state of a given chromosome with a given level of confidence.

The key to one aspect of the present disclosure is the fact that some specialized expert techniques are particularly good at confirming or eliminating from contention certain ploidy states or sets of ploidy states, but may not be good at correctly determining the ploidy state when used alone. This is in contrast to some expert techniques that may be relatively good at differentiating most or all ploidy states from one another, but not with as high confidence as some specialized expert techniques may be at differentiating one particular subset of ploidy states. Some methods use one generalized technique to determine the ploidy state. However, the combination of the appropriate set of specialized expert techniques may be more accurate in making ploidy determinations than using one generalized expert technique.

For example, one expert technique may be able to determine whether or not a target is monosomic with very high confidence, a second expert technique may be able to determine whether or not a target is trisomic or tetrasomic with very high confidence, and a third technique may be able to detect uniparental disomy with very high confidence. None of these techniques may be able to make an accurate ploidy determination alone, but when these three specialized expert techniques are used in combination, they may be able to determine the ploidy call with greater accuracy than when using one expert technique that can differentiate all of the ploidy states reasonably well. In some embodiments of the present disclosure, one may combine the output probabilities from multiple techniques to arrive at a ploidy state determination with high confidence. In some embodiments of the present disclosure, the probabilities that each of the techniques predicts for a given hypothesis may be multiplied together, and that product may be taken to be the combined probability for that hypothesis. The ploidy state(s) associated with the hypothesis that has the greatest combined probability may be called as the correct ploidy state. If the set of expert techniques is chosen appropriately, then the combined product of the probabilities may allow the ploidy state to be determined more accurately than a single technique. In some embodiments of the inversion, the probabilities of the hypotheses from more than one technique may be multiplied, for example using linear algebra, and renormalized, to give the combined probabilities. In one embodiment, the confidences of the probabilities may be combined in a manner similar to the probabilities. In one embodiment of the present disclosure, the probabilities of the hypotheses may be combined under the assumption that they are independent. In some embodiments of the present disclosure, the output of one or more techniques may be used as input for other techniques. In one embodiment of the present disclosure, the ploidy call, made using one or a set of expert techniques, may be used to determine the appropriate input for the allele calling technique. In one embodiment of the present disclosure, the phased, cleaned genetic data output from the allele calling technique may be used as input for one or a set of expert ploidy calling techniques. In some embodiments of the present disclosure, the use of the various techniques may be iterated.

In some embodiments of the present disclosure, the ploidy state may be called with a confidence of greater than about 80%. In some embodiments of the present disclosure, the ploidy state may be called with a confidence of greater than about 90%. In some embodiments of the present disclosure, the ploidy state may be called with a confidence of greater than about 95%. In some embodiments of the present disclosure, the ploidy state may be called with a confidence of greater than about 99%. In some embodiments of the present disclosure, the ploidy state may be called with a confidence of greater than about 99.9%. In some embodiments of the present disclosure, one or a set of alleles may be called with a confidence of greater than about 80%. In some embodiments of the present disclosure, the allele(s) may be called with a confidence of greater than about 90%. In some embodiments of the present disclosure, the allele(s) may be called with a confidence of greater than about 95%. In some embodiments of the present disclosure, the allele(s) may be called with a confidence of greater than about 99%. In some embodiments of the present disclosure, the allele(s) may be called with a confidence of greater than about 99.9%. In some embodiments of the present disclosure, the output allele call data is phased, differentiating the genetic data from the two homologous chromosomes. In some embodiments of the present disclosure, phased allele call data is output for all of the individuals.

Below is a description of several statistical techniques that may be used in the determination of the ploidy state. This list is not meant to be an exhaustive list of possible expert techniques. It is possible to use any statistical technique that is able to place probabilities and/or confidences on the set of ploidy state hypotheses of a target. Any of the following techniques may be combined, or they may be combined with other techniques not discussed in this disclosure.

### Permutation Technique

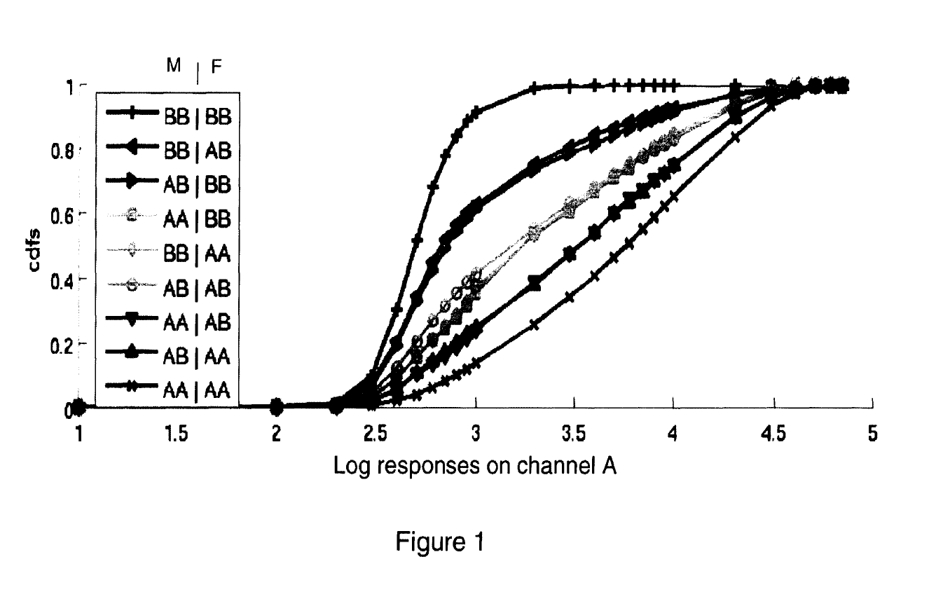

The LDO rate is concordant with the copy number of the genetic material from which it is derived, that is, fewer chromosome copies result in higher LDO, and vice versa. It follows that loci with certain contexts of parental genotypes behave in a characteristic fashion in the embryo, related to the probability of allelic contributions to the embryo. In one embodiment of the present disclosure, called the “permutation technique”, it is possible to use the characteristic behavior of loci in the various parental contexts to infer the ploidy state of those loci. Specifically, this technique involves comparing the relationship between observed distributions of allele measurement data for different parent contexts, and determining which ploidy state matched the observed set of relationships between the distributions. This technique is particularly useful in determining the number of homologous chromosomes present in the sample. By plotting a cumulative distribution function (CDF) curve for each of the parental contexts, one may observe that various contexts cluster together. Note that a CDF curve is only one way to visualize and compare the observed distributions of the allele measurements data. For example, FIG. 1 shows a CDF curve for a disomic chromosome. In particular, FIG. 1 shows how allele measurement data from certain contexts of parental genotypes (Mother|Father) behave in a characteristic fashion in the embryo, related to the probability of allelic contributions to the embryo. The nine parental contexts group into five clusters when the chromosome in question is disomic. On the CDF curve plot, the independent variable, along the x-axis, is the channel response, and the dependent variable, along the y-axis, is the percentage of alleles within that context whose channel response is below a threshold value.

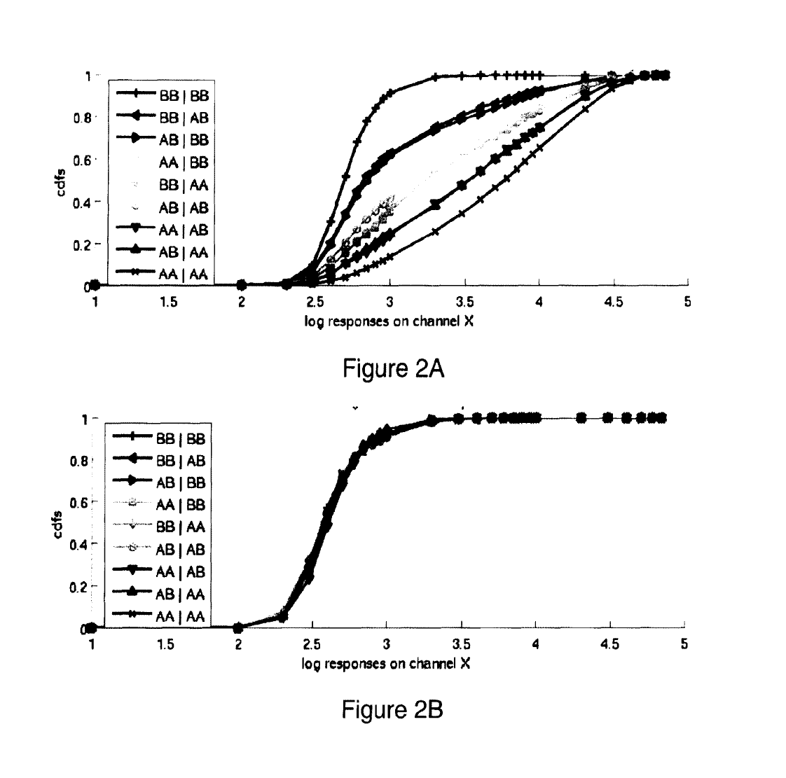

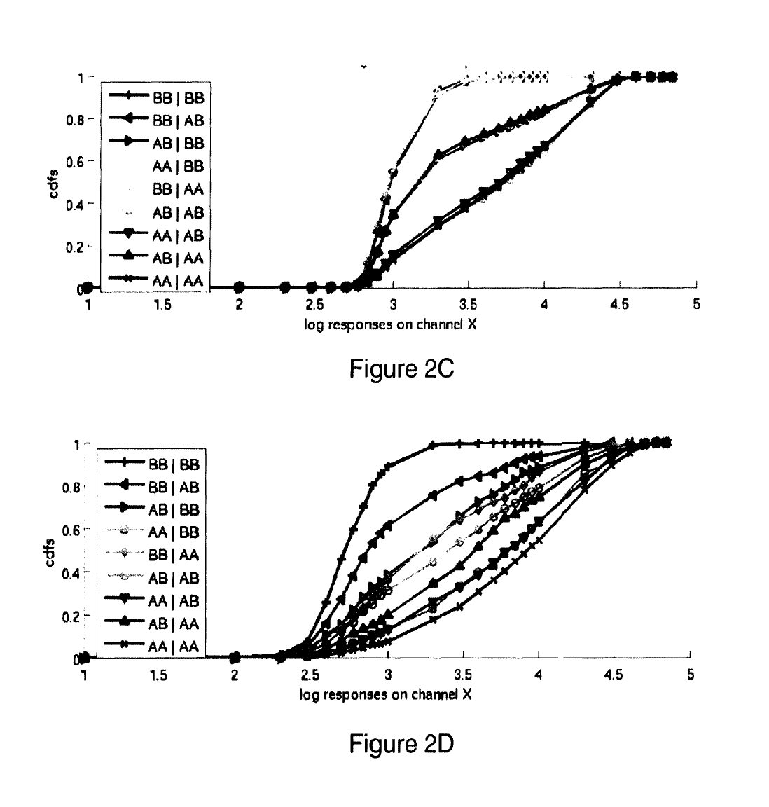

For example, if both parents have homozygous BB states, then the embryo should never have AB or AA states. In this case, measurements on the A detection channel will likely have a distribution determined by background noise and various interference signals, but no valid genotypes. Conversely, if both parents have homozygous AA states, then the embryo should never have AB or BB states, and measurements on the A channel will likely have the maximum intensity possible given the rate of LDO in a particular whole genome amplification. When the underlying copy number state of the embryo differs from disomy, loci corresponding to the specific parental contexts behave in a predictable fashion, based on the additional allelic content that is contributed or is missing from one of the parents. Cumulative density function plots of microarray probe intensity on a detection channel, segregated by parental genotype context, illustrate the concept (see FIG. 2). Specifically, FIGS. 2A-2D show how the relation between the context curves on a CDF plot changes predictably with a change in the chromosome copy number. FIG. 2A shows a cumulative distribution function curve for a disomic chromosome, FIG. 2B shows a cumulative distribution function curve for a nullisomic chromosome, FIG. 2C shows a cumulative distribution function curve for a monosomic chromosome, and FIG. 2D shows a cumulative distribution function curve for a maternal trisomic chromosome.

Each context is represented as MᵢM₂|F₁F₂, where M₁ and M₂ are the maternal alleles, and F₁ and F₂ are the paternal alleles. There are nine possible parental contexts (see FIGS. 2A-2D legend), which, in a disomic chromosome, form five clusters on the CDF plot. In the case of nullosomies, all of the parental context curves cluster with background on the CDF plot. In the case of monosomy, one may expect to see only three context curve clusters, because the removal of one parental context results in only three possible embryonic outcomes: homozygous AA, heterozygous AB, and homozygous BB. One may expect trisomy also to have a distinct CDF-curve topology such that there are seven clusters, caused by extra alleles on a single detection channel and from only one parent.

One set of expected canonical topologies is illustrated in FIGS. 2A-2D, for which the ploidy state may be called by visual inspection of the plots. In some cases, the data from a sample may not be as easy to interpret as the data shown in FIGS. 2A-2D. Many factors may impact data clarity, including: degraded DNA of blastomeres which causes signals with very low signal-to noise ratio; partial ploidy errors which are often encountered during IVF such as translocations; and chromosome-specific and chromosome-segment specific amplification biases possibly caused by the physical positions of the chromosomes in the nucleus or epigenetic phenomenon such as different methylation levels and proteins structures around the chromosomes. These and an assortment of other phenomenon may differentially affect each chromosome of a homologous pair in which case they are difficult to distinguish from ploidy states. In one embodiment of the present disclosure, to accommodate these various affects, a statistical algorithm may be used to analyze data such as that illustrated in FIGS. 2A-2D and generate a ploidy determination together with a confidence in the correctness of that determination.

In one embodiment of the present disclosure, in order to be robust to the differences that may exist between one sample and another, or between cell line samples and blastomeres, the algorithm may be non parametric and does not depend on expected values of statistics or thresholds which are trained on certain samples and applied to others. In one embodiment of the present disclosure, the algorithm uses quantile-rank statistics (a non-parametric permutation method), which first computes the rank of the CDF curve of each context at an intensity at which the background context is within about 80% of a density of about 1. In another embodiment, the algorithm may compute the rank of the CDF curve of each context at an intensity at which the background context is within about 90% of a density of about 1. In another embodiment, the algorithm may compute the rank of the CDF curve of each context at an intensity at which the background context is within about 95% of a density of about 1. Then, the algorithm compares the rank of the data to the expected rank given various ploidy states. For example, if the AB|BB context has the same rank as the BB|AA context, this differs from the expectation under disomy, but is consistent with maternal trisomy. One then may examine the distribution of the data for each sample to determine the probability that two CDF curves could have swapped ranked by random chance, and then use this information, combined with the rank statistics, to determine copy number calls and calculate explicit confidences. The result of this statistical technique is a highly accurate diagnosis of chromosome copy number, combined with an explicit confidence in each call.

Since the permutation technique's copy number call for a given chromosome is independent of all other chromosomes, without loss of generality it is possible to focus on a single given chromosome. For a given maternal genotype gM and paternal genotype gF one may use gM|gF to denote the parental context, e.g. AB|BB refers to the SNPs where the mother's genotype is AB and the father's genotype is BB.

For a given context gM|gF, let X_{gM|gF} denote the set of x-channel responses for all SNPs in the context gM|gF. Similarly, one may use Y_{gM|gF} to denote the set of y-channel responses. Furthermore, for a given positive number C, one may define I_{{x≦c}}

n_{gM|gF}^{x}(c)=Σ_{xεXgM|gF} I_{{x≦c}}and n_{gM|gF}^{y}(c)=Σ_{yεYgM|gF} I_{{y≦c}}

One may also use N_{gM|gF} to denote the number of SNPs in the context gM|gF. It is possible to define

\hat{p}_{gM|gF}^{x}(c)=(n_{gM|gF}^{x}(c))/(N_{gM|gF}) and \hat{p}_{gM|gF}^{y}(c)=(n_{gM|gF}^{y}(c))/(N_{gM|gF})

One can think of \hat{p}_{gM|gF}^{x}(c), \hat{p}_{gM|gF}^{y}(c) as the value of the empirical CDF of the x-channel, y-channel, response of context gM|gF at the point c. One may denote the true CDFs as p_{gM|gF}^{x}(c), and p_{gM|gF}^{y}(c).

### The Algorithm

The main idea behind the algorithm is that for a given positive integer c, the order p_{AA|AA}^{x}(c), p_{AB|AA}^{x}(c), p_{BB|AA}^{x}(c), p_{AA|AB}^{x}(c), p_{AB|AB}^{x}(c), p_{BB|AB}^{x}(c), p_{AA|BB}^{x}(c), p_{AB|BB}^{x}(c), and p_{BB|BB}^{x}(c), will vary based on the chromosome copy number. The same holds for the y-channel. In one embodiment of the present disclosure, one may use this order to determine chromosome copy number. Since the x-channel and y-channel are treated independently, going forward this discussion will focus on only the x-channel.

### Calculations

The first step is to pick a value for c that maximizes distinguishability between the contexts, that is, the value for c which maximizes the difference between the two extreme contexts, AA|AA and BB|BB. More precisely one may define:

$${c}_{x} = \frac{arg max}{\text{?}} \text{?} ( c ) - \text{?} ( c ) and$$

$${e}_{x} = \text{?} ( {c}_{x} ) - \text{?} ( {c}_{x} ) , \\ \text{?} \text{indicates text missing or illegible when filed}$$

and also

$${c}_{y} = \frac{arg max}{\text{?}} \text{?} ( c ) - \text{?} ( c ) and$$

$${e}_{x} = \text{?} ( {c}_{y} ) - \text{?} ( {c}_{y} )$$

$$\text{?} \text{indicates text missing or illegible when filed}$$

This discussion will therefore use cₓ as the sample point and make all order comparisons with regards to \hat{p}_{AA|AA}^{x}(cₓ), \hat{p}_{AB|AA}^{x}(cₓ), \hat{p}_{BB|AA}^{x}(cₓ), \hat{p}_{AA|AB}^{x}(cₓ), \hat{p}_{AB|AB}^{x}(cₓ), \hat{p}_{BB|AB}^{x}(cₓ), \hat{p}_{AA|BB}^{x}(cₓ), \hat{p}_{AB|BB}^{x}(cₓ), and \hat{p}_{BB|BB}^{x}(cₓ). From here forward the discussion will drop the dependence on cₓ. In order to assign a confidence to the chromosome copy number call, it is important to determine a variance for each p_{gM|gF}^{x}. This may be done by making use of a binomial model. In particular, one may observe that each n_{gM|gF}^{x} is the sum of I.I.D. Bernoulli random variables, and hence the normalized sum, has standard deviation

$${σ}_{gM \vert gF}^{x} = \sqrt{\frac{{p}_{gM \vert gF}^{x} ( 1 - {p}_{gM \vert gF}^{x} )}{{N}_{gM \vert gF}}}$$

### Confidence Calculation

Described herein is a method to calculate a confidence on a given copy number hypothesis. Each hypothesis has a set of valid permutation of

| | | |
|---|---|---|
| p_{AA\|AA}^{x} ≈ p_{AA\|AA}^{x} | p_{AA\|AB}^{x} ≈ p_{AA\|AB}^{x} | p_{BB\|AB}^{x} ≈ p_{BB\|AB}^{x} |
| p_{AB\|AA}^{x} ≈ p_{AB\|AA}^{x} | p_{AB\|AB}^{x} ≈ p_{AB\|AB}^{x} | p_{AA\|BB}^{x} ≈ p_{AA\|BB}^{x} |
| p_{BB\|AA}^{x} ≈ p_{AB\|AA}^{x} | p_{BB\|AB}^{x} ≈ p_{BB\|AB}^{x} | p_{AB\|BB}^{x} ≈ p_{AB\|BB}^{x} |

For example, a hypothesis of disomy would have the following set of valid permutations:

| | | |
|---|---|---|
| p_{AA\|AA}^{x} ≈ p_{AA\|AA}^{x}: 1 | p_{AA\|AB}^{x} ≈ p_{AA\|AB}^{x}: 2 | p_{AA\|BB}^{x} ≈ p_{AA\|BB}^{x}: 3 |
| p_{AB\|AA}^{x} ≈ p_{AB\|AA}^{x}: 2 | p_{AB\|AB}^{x} ≈ p_{AB\|AB}^{x}: 3 | p_{AB\|BB}^{x} ≈ p_{AB\|BB}^{x}: 4 |
| p_{BB\|AA}^{x} ≈ p_{AB\|AA}^{x}: 3 | p_{BB\|AB}^{x} ≈ p_{BB\|AB}^{x}: 4 | p_{BB\|BB}^{x} ≈ p_{BB\|BB}^{x}: 5 |

where two entries are given the same value if their relative order is not specified under the hypothesis. Hence there are 12 valid permutations for disomy. Confidence for a given hypothesis is calculated by finding the valid permutation which matches the observed data. This is done by ordering the elements of the invariant groups, groups which have the same order numbers, with regards to their observed statistic.

For example, given that the following order is observed:

$$[ \begin{matrix}{\hat{p}}_{AA \vert AA}^{x} \\ {\hat{p}}_{AB \vert AA}^{x} \\ {\hat{p}}_{BB \vert AA}^{x} \\ {\hat{p}}_{AA \vert AB}^{x} \\ {\hat{p}}_{AB \vert AB}^{x} \\ {\hat{p}}_{BB \vert AB}^{x} \\ {\hat{p}}_{AA \vert BB}^{x} \\ {\hat{p}}_{AB \vert BB}^{x} \\ {\hat{p}}_{BB \vert BB}^{x}\end{matrix} ]$$

the permutation that is consistent with disomy and matches the data is

$$[ \begin{matrix}{p}_{AA \vert AA}^{x} \\ {p}_{AB \vert AA}^{x} \\ {p}_{BB \vert AA}^{x} \\ {p}_{AA \vert AB}^{x} \\ {p}_{AB \vert AB}^{x} \\ {p}_{BB \vert AB}^{x} \\ {p}_{AA \vert BB}^{x} \\ {p}_{AB \vert BB}^{x} \\ {p}_{BB \vert BB}^{x}\end{matrix} ]$$

One may then calculate the probability of the observed x-channel data given a hypothesis of disomy as Pr{x-data|H_{1,1}}=Pr{x-data|best match order}

$${( a )}_{\text{?}} Pr \{ {\hat{p}}_{AA \vert AA}^{x} , {\hat{p}}_{AB \vert AA}^{x} \vert {p}_{AA \vert AA}^{x} \leq {p}_{AB \vert AA}^{x} \} \cdot Pr \{ {\hat{p}}_{AB \vert AA}^{x} , {\hat{p}}_{AA \vert AB}^{x} \vert {p}_{AB \vert AA}^{x} \leq {p}_{AA \vert AB}^{x} \} \cdot Pr \{ {\hat{p}}_{AA \vert AB}^{x} , {\hat{p}}_{BB \vert AA}^{x} , \vert {p}_{AA \vert AB}^{x} \leq {p}_{BB \vert AA}^{x} \} \cdot Pr \{ {\hat{p}}_{BB \vert AA}^{x} , {\hat{p}}_{AA \vert BB}^{x} \vert {p}_{BB \vert AA}^{x} \leq {p}_{AA \vert BB}^{x} \} \cdot Pr \{ {\hat{p}}_{AA \vert BB}^{x} , {\hat{p}}_{AB \vert AB}^{x} \vert {p}_{AA \vert BB}^{x} \leq {p}_{AB \vert AB}^{x} \} \cdot Pr \{ {\hat{p}}_{AB \vert AB}^{x} , {\hat{p}}_{BB \vert AB}^{x} \vert {p}_{AB \vert AB}^{x} \leq {p}_{BB \vert AB}^{x} \} \cdot Pr \{ {\hat{p}}_{BB \vert AB}^{x} , {\hat{p}}_{AB \vert BB}^{x} \vert {p}_{BB \vert AB}^{x} \leq {p}_{AB \vert BB}^{x} \} \cdot Pr \{ {\hat{p}}_{AB \vert BB}^{x} , {\hat{p}}_{BB \vert BB}^{x} \vert {p}_{AB \vert BB}^{x} \leq {p}_{BB \vert BB}^{x} \}$$

$$\text{?} \text{indicates text missing or illegible when filed}$$

In this case, the approximation (a) is made in order to make the probability computable. Finally, for any two contexts gM1|gF1 and gM2|gF one may calculate:

$$Pr \{ {\hat{p}}_{gM 1 \vert g F 1}^{x} , {\hat{p}}_{gM 2 \vert g F 2}^{x} \vert {p}_{gM 1 \vert g F 1}^{x} \leq {p}_{gM 2 \vert g F 2}^{x} \} = \frac{1}{\text{?}} Pr \{ {\hat{p}}_{gM 1 \vert g F 1}^{x} , {\hat{p}}_{gM 2 \vert g F 2}^{x} , {p}_{gM 1 \vert g F 1}^{x} \leq {p}_{gM2 \vert g F 2}^{x} \} {=}^{( c )} \frac{1}{\text{?}} \cdot \int \text{?} Pr \{ {\hat{p}}_{gN 1 \vert g F 1}^{x} , {\hat{p}}_{gN 2 \vert g F 2}^{x} , {p}_{gM 1 \vert g F 1}^{x} , {p}_{gM 2 \vert g F 2}^{x} \} d {p}_{gM 1 \vert g F 1}^{x} d {p}_{gM 2 \vert g F 2}^{x} {=}^{( b )} α \int \text{?} Pr \{ {\hat{p}}_{gM 1 \vert g F 1}^{x} , {\hat{p}}_{gM 2 \vert g F 2}^{x} \vert {p}_{gM 1 \vert g F 1}^{x} , {p}_{gM 2 \vert g F 2}^{x} , \} d {p}_{gM 1 \vert g F 1}^{x} d {p}_{gM 2 \vert g F 2}^{x} {=}^{( c )} α \int \text{?} f \text{?} f \text{?} = α \int \text{?} f \text{?} f \text{?} d \text{?} d \text{?}$$

$$\text{?} \text{indicates text missing or illegible when filed}$$

where (a) and (b) follow from independence and an assumption of a uniform distribution on the p_{gM|gF}^{x} and (c) follows from the use of to denote the normal PDF with mean μ and standard deviation σ and an application of the CLT. Finally from (1) it is possible to derive:

Pr{\hat{p}_{gM1|gF1}^{x},\hat{p}_{gM2|gF2}^{x}|p_{gM1|gF1}^{x}≦p_{gM2|gF2}^{x}}=Pr{W₁≦W₂}, where

W₁˜N(p_{gM1|gF1}^{x},σ_{gm|gF1}^{x}) and

W₂˜N(p_{gM2|gF2}^{x},σ_{gM2|gF2}^{x})

The confidences from the x-channel and y-channel are combined under the assumption of independence, i.e.

Pr{data|H_{1,1}}=Pr{x-data|H_{1,1}}Pr{y-data|H_{1,1}}.

In this manner it is possible to calculate the probability of the data given each hypothesis. In one embodiment, Bayes' rule may be used to find the probability of each hypothesis given the data.

### Nullsomy

In one embodiment of the present disclosure, when using the permutation technique, nullsomies are handled in a special way. In addition to assigning a confidence assigned to the the copy number call, it is also possible to perform an envelope test. If the envelope ex or e_{y} is less than a threshold the probability of nullsomy is set to about 1 and the probability of the other hypotheses is set to about 0. In one embodiment of the present disclosure, this threshold may be set to about 0.05. In one embodiment of the present disclosure, this threshold may be set to about 0.1. In one embodiment of the present disclosure, this threshold may be set to about 0.2. The nullsomy permutation set for the x-channel is as follows:

| | | |
|---|---|---|
| p_{AA\|AA}^{x} ≧ p_{BB\|BB}^{x} | p_{AB\|AA}^{x} ≧ p_{BB\|BB}^{x} | p_{AA\|AB}^{x} ≧ p_{BB\|BB}^{x} |
| p_{AA\|AA}^{x} ≧ p_{BB\|AB}^{x} | p_{AB\|AA}^{x} ≧ p_{BB\|AB}^{x} | p_{AA\|AB}^{x} ≧ p_{BB\|AB}^{x} |
| p_{AA\|AA}^{x} ≧ p_{AB\|BB}^{x} | p_{AB\|AA}^{x} ≧ p_{BB\|AB}^{x} | p_{AA\|AB}^{x} ≧ p_{AB\|BB}^{x} |

where the order of all contexts not listed are chosen to maximize the probability. Similarly, the nullsomy permutation set for the y-channel is as follows:

| | | |
|---|---|---|
| p_{BB\|BB}^{y} ≧ p_{AA\|AA}^{y} | p_{AB\|BB}^{y} ≧ p_{AA\|AA}^{y} | p_{BB\|AB}^{y} ≧ p_{AA\|AA}^{y} |
| p_{BB\|BB}^{y} ≧ p_{AA\|AB}^{y} | p_{AB\|BB}^{y} ≧ p_{AA\|AB}^{y} | p_{BB\|AB}^{y} ≧ p_{AA\|AB}^{y} |
| p_{BB\|BB}^{y} ≧ p_{AB\|AA}^{y} | p_{AB\|BB}^{y} ≧ p_{AB\|AA}^{y} | p_{BB\|AB}^{y} ≧ p_{AB\|AA}^{y} |

### Segmentation

The standard permutation algorithm described above works well in a majority of the cases and gives theoretical confidences which correspond to empirical error rates. The one issue that has arisen is regional specific behavior in a small subset of the chromosome data. This behavior may be due to proteins blocking some sections of the chromosomes, or a translocation. To handle such regional issues, it is possible to use a segmented protocol interface to the permutation method.

If a chromosome is given a confidence below a threshold, the chromosome is broken down into a number of regions and the segmentation algorithm is run on each segment. In one embodiment of the present disclosure, about five equal segments are used. In one embodiment of the present disclosure, between about two and about five segments may be used. In one embodiment between about six and about ten segments may be used. In one embodiment of the present disclosure, more than about ten segments may be used. In one embodiment of the present disclosure, this threshold may be set to about 0.6. In one embodiment of the present disclosure, this threshold may be set to about 0.8. In one embodiment of the present disclosure, this threshold may be set to about 0.9. Then, one may focus on the segments which are assigned confidences greater than a threshold and try to find a majority vote among these high confidence segments. In one embodiment of the present disclosure, this threshold may be set to about 0.5. In one embodiment of the present disclosure, this threshold may be set to about 0.7. In one embodiment of the present disclosure, this threshold may be set to about 0.8. For example, in the case where five equal segments are used, if no majority of three or greater exists the technique may output the standard permutation algorithms confidences, while if a majority of three or more high confidence segments does exist, these segments may be pooled together and the standard permutation algorithm is run on the pooled data. The technique may then output the confidences on the pooled data as the confidence for the whole chromosome.

In one embodiment of the present disclosure, if one of the minority segments has confidence greater than a threshold, that chromosome may be flagged as being segmented. In one embodiment of the present disclosure, this threshold may be set to about 0.8. In one embodiment of the present disclosure, this threshold may be set to about 0.9. In one embodiment of the present disclosure, this threshold may be set to about 0.95

### Whole Chromosome Mean

In some cases, different chromosomes may have different amplification profiles. In one embodiment of the present disclosure, it is possible to use the following technique, termed the “whole chromosome mean” technique to increase the accuracy of the data by correcting or partly correcting for this amplification bias. This technique also serves to correct or partly correct for any measurement or other biases that may be present in the data. This technique does not rely on the identity of any of the alleles as measured by various genotyping techniques, rather, it relies only on the overall intensity of the genotyping measurements. Typically, the raw output data from a genotyping technique, such as a genotyping array, is a set of measured intensities of the channels that correspond to each of the four base pairs, A, C, G and T. These measured intensities, taken from the channel outputs, are designed to correlate with the amount of genetic material present, thus the base pair whose measured intensity is the greatest is often taken to be the correct allele. In some embodiments, the measured internisities for certain sets of SNPs are averaged, and the characteristic behavior of those means are used to determine the ploidy state of the chromosome.

The first step is to normalize each target for variation in amplification. This is done by using an alternate method to make an initial determination of ploidy state. Then, one selects all chromosomes with a ploidy call with a confidence greater than a certain threshold. In one embodiment of the present disclosure, this threshold is set at approximately 99%. In one embodiment of the present disclosure, this threshold is set at approximately 95%. In one embodiment of the present disclosure, this threshold is set at approximately 90%. Then, the adjusted means of the selected chromosomes are used as a measure of the overall amplification of the target. In one embodiment of the present disclosure, only the intensity of the fluorescent probe, averaged over the whole chromosome, is used. In one embodiment, the intensities of the genotyping output data, averaged over a set of alleles, is used.

Then the means are adjusted with respect to the copy number call of the chromosome, normalizing with respect to a disomy, i.e. monosomies are scaled by 2, disomies by 1 and trisomies by ⅔. The means of each chromosome of the target are then divided by the mean of these high confidence adjusted means. These normalized means may be referred to as the amplification adjusted means. In one embodiment, only the channel outputs alleles from certain contexts are used. In one embodiment, only the alleles from AA|AA or BB|BB are used.

Once the targets have been normalized for amplification variations, each chromosome may be normalized for chromosome specific amplification variance. For the k^{th} chromosome find all targets which have chromosome k called disomy with confidence greater than the threshold confidence. Take the mean of their amplification adjusted means. This will serve as the average amplification of chromosome k, which may be referred to as b{k}. Without loss of generality, set b{1} to 1 by dividing out all other b{k} by b{1}.

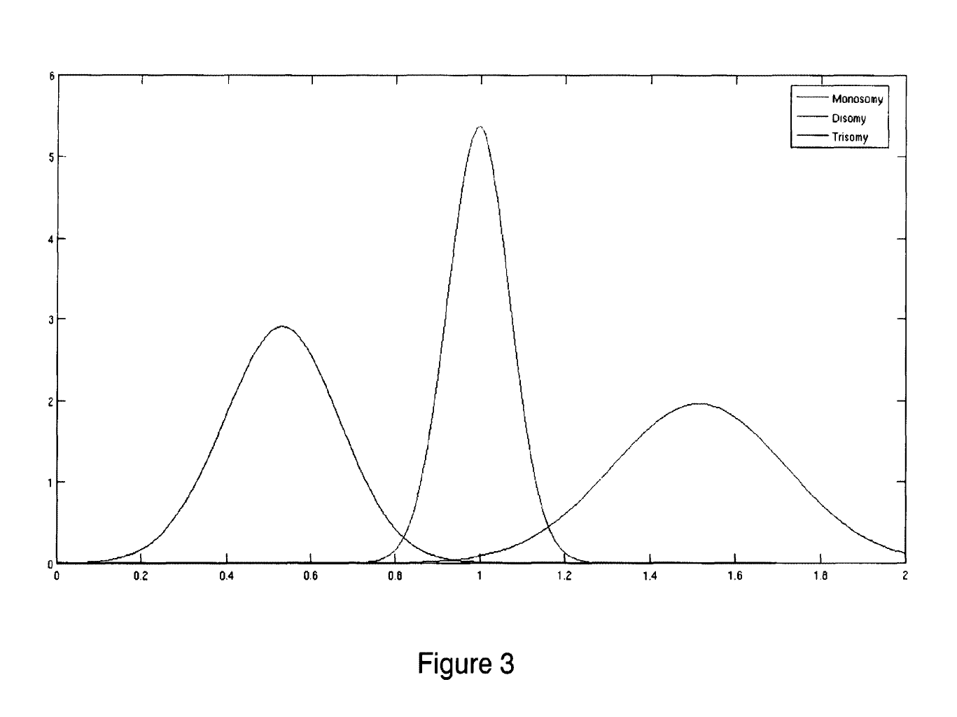

The amplification normalized means may be normalized for chromosome variation by dividing out by the vector [b{1}, . . . , b{24}]. These means are referred to as the standardized means. From a training set made up of historical data, it may be possible to find means and standard deviations for these standardized means under the assumptions of monosomy, disomy and trisomy. These standardized means, under the various ploidy state assumptions, may be taken to be expected intensities for comparative purposes. In one embodiment, a probability may be calculated using statistical methods known to those skilled in the art, and using the measured mean intensities of the genotyping output data, and the expected mean intensities of the genotyping output data. A probability for each of the ploidy state hypotheses may be calculated under a Gaussian hypothesis or through a non-parametric method such as a kernel method for density estimation. Then pool all data with a given ploidy call and confidence greater than a certain threshold. In one embodiment, the threshold is approximately 80%. In one embodiment, the threshold is approximately 90%. In one embodiment, the threshold is approximately 95%. Assuming Gaussian distributions, the output should be a set of hypothesis distributions. FIG. 3 shows a hypothesis distribution of monosomy (left), disomy (middle), and trisomy (right) using the Whole Chromosome Mean technique and using internal historical data as training data.

In the first step of the whole chromosome means method, each target may be normalized for amplification variation. This may be done without first normalizing for chromosome variation. In one embodiment of the present disclosure, after one calculates the [b{1}, . . . , b{24}] vector from the amplification normalized means, the vector may be used to adjust the means used to determine the amplification of the target. This will result in new amplification normalized means and hence a new [b{1}, . . . , b{24}] vector. One can iterate this until reaching a fixed point.

### Presence of Parents Technique

In one embodiment of the present disclosure, one may use an expert statistical technique termed the “Presence of Parents” (POP) technique, described in this section, that is particularly good at differentiating any hypotheses that involve no contribution from one or more parents (i.e. nullsomy, monosomy, and uniparental disomy) from those that do. The statistical technique described in this section can detect, independently for each parent, for a given chromosome, whether or not there is a contribution from that parent's genome. The determination is made based on distances between sets of contexts at the widest point on the CDF curves. The technique assigns probabilities to four hypotheses: {both parents present, neither parent present, only mother, only father}. The probabilities are assigned by calculating a summary statistic for each parent and comparing it to training data models for the two cases of “present” and “not present”.

### Calculation of Summary Statistic

The POP algorithm is based on the idea that if a certain parent has no contribution, then certain pairs of contexts should behave identically. The summary statistic X^{p} for parent p on a single chromosome is a measure of the distance between those context pairs. In one embodiment of the present disclosure, on an arbitrary chromosome, five context distances d_{c}^{p1} through d_{c}^{p2} may be defined for each channel cεX, Y and each parent pε{father, mother}. AABB_{X} is defined as the value of the AABB context CDF curve on the X channel measured at the widest envelope width, and so on.

d_{c}^{m1}=AABB_{c}−BBBB_{c}

d_{c}^{m2}=AABB_{c}−BBBB_{c}

d_{c}^{m3}=AAAB_{c}−BBAB_{c}

d_{c}^{m4}=AAAA_{c}−BBAA_{c}

d_{c}^{m5}=AAAA_{c}−ABAA_{c}

When there is no contribution from the mother, all ten of {d_{c}^{mi}} should be zero. When there is a contribution from the mother, the set of five {d_{X}^{mi}} should be negative and the set of five {d_{Y}^{mi}} should be positive. Similarly, ten distances d_{c}^{f1} . . . d_{c}^{f5} may be defined for the father, and should be zero when the father's contribution is not present.

d_{c}^{f1}=BBAB_{c}−BBBB_{c}

d_{c}^{f2}=BBAA_{c}−BBBB_{c}

d_{c}^{f3}=ABAA_{c}−ABBB_{c}

d_{c}^{f4}=AAAA_{c}−AAAB_{c}

d_{c}^{f5}=AAAA_{c}−AABB_{c}

Each distance may be normalized by the channel envelope width to form the i^{th} normalized distance g_{c}^{pi} for parent p on channel c. The envelope width is also measured at its widest point.

g_{c}^{pi}=d_{c}^{pi}/abs(AAAA_{c}−BBBB_{c})

A single statistic for parent p on the current chromosome is formed by summing the normalized distances over the five context pairs i and both channels.

X^{p}=Σᵢ₌₁^{g}g_{Y}^{pi}−Σᵢ₌₁^{g}g_{X}^{pi}

### Training Distributions

Having calculated a statistic Xp for each parent on a given chromosome, it can be compared to distributions for the cases of “parent present” and “parent not present” to calculate the probability of each.

In one embodiment of the present disclosure, the training data distributions may be based on a set of blastomeres that have been filtered using one or a combination of other copy number calling techniques. In one embodiment of the present disclosure, hypothesis calls from both the permutation technique and the WCM are considered, with nullsomy detected using the minimum required envelope width criterion. In one embodiment, to be included in the training data, a chromosome must be called with high confidence. In one embodiment of the present disclosure, this confidence may be set at about 0.6. In one embodiment of the present disclosure, this confidence may be set at about 0.8. In one embodiment of the present disclosure, this confidence may be set at about 0.9. In one embodiment of the present disclosure, this confidence may be set at about 0.95. Chromosomes with high confidence calls of paternal monosomy or paternal uniparental disomy are included in the “mother not present” data set. Non-nullsomy chromosomes with high confidence calls on all other hypotheses are included in the “mother present” data set, and the father data sets are constructed similarly.

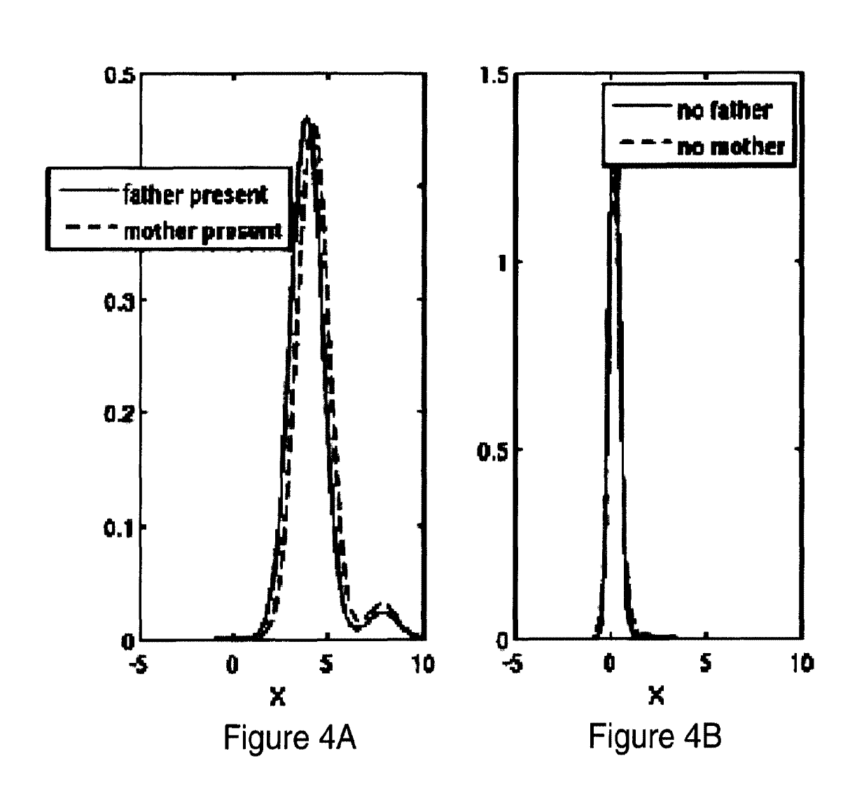

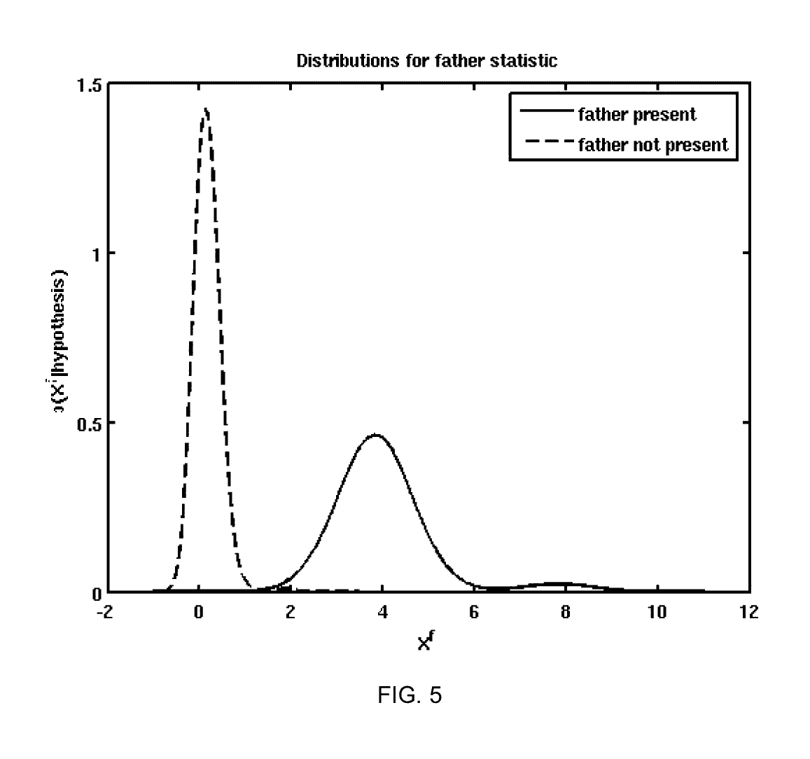

In one embodiment of the present disclosure, a kernel density may be formed from each data set, resulting in four distributions on X A wide kernel width is used when the parent is present and a narrow kernel width is used when the parent is not present. In one embodiment of the present disclosure the wide kernel width may be about 0.9, 0.8 or 0.6. In one embodiment of the present disclosure, the narrow kernel width may be about 0.1, 0.2, or 0.4. Several examples of the resulting statistic distributions for the Presence of Parents techniques are shown in FIGS. 4A-4B. FIG. 4A shows a distribution of genetic data of each of the parents when genetic data from the parents are present; FIG. 4B shows a distribution when genetic data from each parent is absent. Note that the “present” distributions (left) are multimodal, representing the scenarios of “one copy present” and “two copies present”. The present and not-present distributions for the father statistic are shown on the same plot in FIG. 5, emphasizing that X^{f} can be used to reliably distinguish between the two cases.

### Hypothesis Probabilities

Hypothesis probabilities for a chromosome are calculated by comparing the representative statistics X^{m} and X^{f} to the training data distributions. The m mother-present statistic provides the likelihood functions m=p(X^{m}|mother present) and m̅=p(X^{m}|mother not present) and the father-present statistic provides the likelihood functions f=p(X^{f}|father present) and f̅=p(X^{f}|father not present). Considering the presence of the mother and father to be independent, the joint likelihood of a hypothesis on both parents can be calculated by multiplication of the individual parent likelihoods. Therefore, the usual hypotheses probabilities structure containing nine likelihoods p(data hypothesis) for parent copy numbers ranging from zero to two can be constructed as shown in Table 1.

TABLE 1
Probability of data given hypothesis, combining mother and father

| | 0 father | 1 father | 2 father |
|---|---|---|---|
| 0 mother | m̅f̅ | m̅f | m̅f |
| 1 mother | mf̅ | mf | mf |
| 2 mother | mf̅ | mf | mf |

### Presence of Homologs Technique

This algorithm, termed the “Presence of Homologs” (POH) technique, makes use of phased parent genetic information, and is able to distinguish between heterogeneous genotypes. Genotypes where there are two identical chromosomes are difficult to detect when using an expert technique that focuses on allele calls. Detection of individual homologs from the parent is only possible using phased parent information. Without phased parent information, only parent genotypes AA, BB, or AB/BA (heterozygous) can be identified. Parent phase information distinguishes between the heterozygous genotypes AB and BA. The POH algorithm is based on the examination of SNPs where the parent of interest is heterozygous and the other parent is homozygous, such as AA|AB, BB|AB, AB|AA or AB|BB. For example, the presence of a B in the blastomere on a SNP where the mother is AB and the father is AA indicates the presence of M₂. Because single-cell data is subject to high noise and dropout rates, the chromosome is segmented into non-overlapping regions and hypotheses are evaluated based on statistics from the SNPs in a region, rather than individually.

Mitotic trisomy is often hard to differentiate from disomy, and some types of uniparental disomy, where two identical chromosomes from one parent are present, is often difficult to differentiate from monosomy. Meiotic trisomy is distinguished by the presence of both homologs from a single parent, either over the entire chromosome in the case of meiosis-one (M1) trisomy, or over small sections of the chromosome in the case of meiosis-two (M2) trisomy. This technique is particularly useful for detecting M2 trisomy. The ability to differentiate mitotic trisomy from meiotic trisomy is useful, for example, the detection of mitotic trisomy in blastomere biopsied from an embryo indicates reasonable likelihood that the embryo is mosaic, and will develop normally, while a meiotic trisomy indicates a very low chance that the embryo is mosaic, and the likelihood that it will develop normally is lower. This technique is particularly useful in differentiating mitotic trisomy, meiotic trisomy and uniparental disomy. This technique is effective in making correct copy number calls with high accuracy.

The presence of a single parent homolog in the embryo DNA can be detected by examining that homolog's indicator contexts. A homolog's indicator contexts (one on each channel) may be defined as the contexts where a signal on that context can only come from that particular homolog. For example, the mother's homolog 1 (M₁) is indicated on channel X in context AB|BB and on channel Y in context BA|AA.

In one embodiment of the present disclosure, the structure of the algorithm is as follows:
(1) Phase parents and calculate noise floors per chromosome
(2) Segment chromosomes
(3) Calculate SNP dropout rates per segment for each context of interest
(4) Calculate allele dropout rate (ADO) for each parent on each target chromosome and the hypothesis likelihoods on each segment
(5) Combine across segments to produce probability of data given parent strand hypothesis for whole chromosome
(6) Check for invalid calls and then calculate outputs

### Parent Phasing and Noise Floor Calculation

The phasing of the parent can be accomplished with a number of techniques. In one embodiment of the present disclosure, the parental genetic data is phased using a method disclosed in this document. In one embodiment of the present disclosure, it may require about 2, 3, 4, 5 or more embryos. In some embodiments of the present disclosure, the chromosome may be phased in segments such that phasing between one segment and another may not be consistent. The phasing method may distinguish genotypes AB and BA with a reported confidence. In one embodiment of the present disclosure, SNPs which are not phased with the required minimum confidence are not assigned to either context. In one embodiment of the present disclosure, the minimum allowed phase confidence is about 0.8. In one embodiment of the present disclosure, the minimum allowed phase confidence is about 0.9. In one embodiment of the present disclosure, the minimum allowed phase confidence is about 0.95.

The noise floor calculation may be based on a percentile specification. In one embodiment of the present disclosure, the percentile specification is about 0.90, 0.95 or 0.98. In one embodiment of the present disclosure, the noise floor on channel X is the 98th percentile value on the BBBB context, and similarly on channel Y. A SNP may be considered to have dropped out if it falls below its channel noise floor. A distinct noise floor may be calculated for each target, chromosome, and channel.

### Chromosome Segmentation

Segmentation of chromosomes, that is, running the algorithm on segments of a chromosome instead of a whole chromosome, is a part of this technique because the calculations are based on dropout rates, which are calculated over segments. Segments which are too small may not contain SNPs in all required contexts, especially as phasing confidence decreases. Segments which are too big are more likely to contain homolog crossovers (ie change from M₁ to M₂) which may be mistaken for trisomy. Because allele dropout rates may be as high as about 80 percent, many SNPs may be required in a segment in order to confidently distinguish allele dropout from the lack of a signal, that is, where the expected dropout rate is about or above 95 percent).

Another reason the segmentation of chromosomes may be beneficial to the technique is that it allows the technique to be executed more quickly with a given level of computational speed and power. Since the number of hypotheses, and thus the calculational needs of the technique, scale roughly as the number of alleles under consideration raised to the nth power, where n is the number of related individuals, reducing the number of alleles under consideration can significantly improve the speed of the algorithm. Relevant segments can be spliced back together after they have been phased.

In one embodiment of the present disclosure, the phasing method segments each chromosome into regions of 1000 SNPs before phasing. The resulting segments may have varying numbers of SNPs phased above a given level of confidence. In one embodiment of the present disclosure, the algorithm's segments used for calculating dropout rates may not cross boundaries of phasing segments because the strand definitions may not be consistent. Therefore, segmentation is accomplished by subdivision of the phasing segments. In one embodiment between about 2 and about 4 segments are used for a chromosome. In one embodiment between about 5 and about 10 segments are used for a chromosome. In one embodiment between about 10 and about 20 segments are used for a chromosome. In one embodiment between about 20 and about 30 segments are used for a chromosome. In one embodiment between about 30 and about 50 segments are used for a chromosome. In one embodiment more than about 50 segments are used for a chromosome.

In one embodiment of the present disclosure, approximately 20 segments are used on large chromosomes and approximately 6 segments are used on very small chromosomes. In one embodiment of the present disclosure the number of segments used is calculated for each chromosome, ranging from about 6 to 20, and varies linearly with the total number of SNPs on the chromosome. In one embodiment of the present disclosure, if the number of phasing segments is greater or equal to the desired number of segments, the phasing segments are used as is, and if not, the phasing segments are uniformly subdivided into n segments each, where n is the minimum required to reach the desired number of segments.

### Calculation of Dropout Rates

The data on a particular chromosome segment is summarized by the dropout rates on a set of contexts. Dropout rate may be defined, for this section, as the fraction of SNPs on the given context (with its specified channel) which measure below the noise floor. Six contexts may be measured for each parent. The dropout rates âₓ and â_{y} may reflect the allele dropout rate, and the dropout rates ŝₓⁱ and ŝ_{y}ⁱ may indicate the presence of homolog i. The following table, Table 2, shows an example of the contexts associated with each dropout rate for each parent. The measured dropout rate and the number of SNPs for each context must be stored. Note that each of the three dropout rates in the Table 2 are measured on two different contexts for each parent.

TABLE 2
Contexts for required dropout rates

| | mom, X | mom, Y | dad, X | dad, Y |
|---|---|---|---|---|
| â | AABB | BBAA | BBAA | AABB |
| ŝ¹ | ABBB | BAAA | BBAB | AABA |
| ŝ² | BABB | ABAA | BBBA | AAAB |

### Maximum Likelihood Estimation of ADO

This section contains a discussion of a method to estimate the allele dropout rate a* for each parent on each target, based on likelihoods of the form p(Dₛ|Mᵢ, a) and p(Dₛ|Fᵢ, a). The ADO may be defined as the probability of signal dropout on an AB SNP. Dₛ may be defined as the set of context dropout rates measured on a segment of a chromosome and Mᵢ, Fᵢ, are the parent strand hypotheses. In one embodiment of the present disclosure, calculations are performed using log likelihoods due to the relatively small probabilities generated by multiplication across contexts and segments.

The allele dropout rate may be estimated using a maximum likelihood estimate calculated by brute force grid search over the allowable range. In one embodiment of the present disclosure, the search range [aₘᵢₙ, aₘₐₓ] may be set to about [0.4; 0.7]. At high levels of ADO, it becomes difficult to distinguish between presence and absence of a signal because the ADO approaches the noise threshold dropout rate of about 0.95.

In one embodiment of the present disclosure, the allele dropout rate is calculated for a particular target, for each parent, using the following algorithm. In one embodiment of the present disclosure, the calculation may be performed using matrix operations rather than for each target and chromosome individually.

for aε[aₘᵢₙ, aₘₐₓ]

for chε[1, 22] (22 chromosomes)

Calculate P(Dₛ|Mᵢ, a) ∀I, ∀s on chromosome

Mₛⁿ=arg max P(Dₛ|Mᵢ,a) (maximize over hypotheses on each segment)

P(D_{ch}|Mₛⁿ,a)=ΠₛP(Dₛ|Mₛⁿ,a) (combine across segments on chromosome)

Λ(a)=Π_{ch}P(D_{ch}|Mₛⁿ,a) (combine across chromosomes)

a*=arg max Λ(a) (optimize over a)

### Modeling Data Likelihoods

In one embodiment of the present disclosure, the ADO optimization may utilize a model for dropout rate on various contexts as a function of parent strand hypothesis and ADO. SNP dropouts on a single chromosome segment may be considered I.I.D. Bernoulli variables, and the dropout rate would be expected to be normally distributed with mean μ and standard deviation σ=\sqrt{μ(1−μ)/N} where N is the number of SNPs measured. The dropout rate model may calculate μ as a function of the hypothesis, ADO, and context. The hypothesis and context together determine a genotype for a SNP, such as AB. The genotype and the ADO rate then determine μ. In one embodiment of the present disclosure, the hypotheses for the mother are {M₀,M₁,M₂,M₁₂,M₁₁,M₂₂}. Other sets of hypotheses may be equally well used. M₀ means that no homolog from the mother is present. M₁₁ and M₂₂ are cases where two identical copies from the mother are present. These do not indicate meiotic trisomy. The hypotheses consistent with disomy are M₁ and M₂.

Table 3 lists μ? by mother hypothesis and the various dropout rate measurements in this embodiment of the present disclosure. The identical table may be used for corresponding father strand hypotheses. Recall that p is the dropout rate which defines the noise floor, and is therefore the expected dropout rate for a channel with no allele present.

TABLE 3
Expected segment dropout rate model by strand hypothesis

| | M₀ | M₁ | M₂ | M₁₂ | M₁₁ | M₂₂ |
|---|---|---|---|---|---|---|
| \hat{α} | p | a | a | α² | α² | α² |
| ŝ¹ | p | a | p | a | α² | p |
| ŝ² | p | p | a | a | p | α² |

On each segment, the three dropout rates â, ŝ¹ and ŝ² are measured on both channels. Thus, the total data Dₛ from a segment consists of 6 dropout rate measurements, and the likelihood P(Dₛ|Mᵢ, a) is the product of the 6 corresponding probabilities under the normal distributions determined by μ from Table 3.

Because identification of SNPs for the ŝ¹ and ŝ² dropout rates depends on parent phasing, there may not be any identified SNPs in some contexts. Each of the three measured dropout rates â, ŝ¹ and ŝ² may be measured on two different contexts corresponding to the two channels. If any of the three has no data in either of its contexts, then likelihoods for that segment may be not calculated. Chromosomes which have been called nullsomy by the standard envelope width test may be not included.

### Calculate Chromosome Likelihoods by Combining Segments

The likelihood calculations described above provide a data likelihood P(Dₛ|Mᵢ) on each segment s for each parent strand hypothesis Mᵢ. The two parents may still considered independently. The strand likelihoods may then be normalized so that the sum of all likelihoods on a single segment is one. The normalized likelihoods from segment s will be referred to as {Λₛ(Mᵢ)}. This process will also depend on the normalized segments lengths {xₛ}, defined as the fraction of a chromosome's SNPs contained on segment s.

In one embodiment of the present disclosure, the likelihoods from all segments may now be combined to form a set of chromosome likelihoods for the number of distinct strands present. All of the data for a chromosome is combined into D_{ch}. The chromosome hypotheses are S₀^{m}, S₁^{m}, S₂^{m} for the mother. S₁^{m} is the hypotheses that only one distinct homolog is present at a time, which allows the strand hypotheses M₁; M₁₁; M₂; M₂₂. S₂^{m} r is the meiotic trisomy hypotheses, where two distinct strands have been contributed from the mother. Hypotheses on the mother's strand number will be discussed; hypotheses on the father's strand may be calculated in an analogous fashion.

S₀^{m} corresponds one-to-one with the no-strand hypothesis M₀. Therefore, the likelihood of no-copies is simply the sum (weighted by segment length) of the no-strand likelihoods on each segment.

P(D_{ch}|S₀^{m})=ΣₛΛₛ(M₀)xₛ

S₁^{m} (one copy at a time) corresponds to the strand hypotheses M₁; M₁₁; M₂; M₂₂. Without making any assumptions about recombination, one may expect that a single parent copy will be either M1 or M2 strand at all segments. In this embodiment of the present disclosure, rather than trying to detect how many copies of a single strand are present, the double-strand hypotheses M₁₁ and M₂₂ are included as well. In another embodiment of the present disclosure, M₁ and M₂ may be grouped into one hypothesis, and M₁₁ and M₂₂ may be grouped in another hypothesis. In other embodiments, other hypotheses may refer to other groupings of the actual state of the genetic material. Again, the chromosome likelihood is simply a weighted sum.

P(D_{ch}|S₁^{m})=Σₛ(Λₛ(M₁)+Λₛ(M₁₁)+Λₛ(M₂)+Λₛ(M₂₂))xₛ

Meiotic trisomy is characterized by the presence of two non-identical chromosomes from a single parent. Depending on the type of meiotic error, these may be a complete copy of each of the parent's homologs (meiosis-1), or they may be two different recombinations of the parent's homologs (meiosis-2). The first case results in strand hypothesis M₁₂ on all segments, but the second case results in M₁₂ only where the two different combinations don't match. Therefore, the weighted sum approach used for the other hypotheses may not be appropriate.

The meiotic trisomy likelihood calculation is based on the assumption that unique recombinations will be distinct on at least one continuous region covering at least a quarter of the chromosome. In other embodiments, other sizes for the continuous region on which unique recombinations are distinct may be used. A detection threshold that is too low may result in trisomies being incorrectly called due to mid-segment recombinations and noise. Because meiosis-2 trisomy does not correspond to any whole-chromosome strand hypothesis, the likelihood may not be proportional to the sum of segment likelihoods as it is for the other two copy numbers. Instead, the confidence on the meiotic hypothesis depends on whether or not the meiotic threshold has been met, and the overall confidence of the chromosome.

In one embodiment of the present disclosure, the chromosomes may be reconstructed by recombining the segments along with their relative probabilities using the following steps:

1. Find length x of longest continuous region with Λ(M₁₂)>0:8 by combining adjacent segments
2. If x>0:25 then set the meiotic flag as true. Otherwise set the flag as false.
3. Calculate general confidence on chromosome by averaging confidence on most likely hypothesis from each segment C=Σₛ xₛmaxΛₛ(Mᵢ)4. If the meiotic flag is true, then let the normalized P(D_{ch}|S₂^{m})=C. Otherwise let P(D_{ch}|S₂^{m})=1−C.

The result is that if the meiotic flag is triggered on a high confidence chromosome, the meiotic hypothesis will have correspondingly high confidence. If the meiotic flag is not triggered, the meiotic hypothesis will have low confidence.

### Check for Invalid Calls and Calculate CNC Outputs

The final step is to calculate likelihoods on true parent copy numbers without distinction between meiotic and mitotic error. The standard HNₘN_{f} notation will be adapted for single parents, where Nₘ is the number of strands from the mother present, and N_{f} is the number of strands from the father present.

P(D_{ch}|H0x)=P(D_{ch}|S₀^{m})

P(D_{ch}|H1x)=P(D_{ch}|S₁^{m})

P(D_{ch}|H2x)=P(D_{ch}|S₂^{m})P(meiotic)+P(D_{ch}|S₁^{m})P(mitotic)

The final formula is explained by the fact that trisomy can arise due to two disjoint events: meiotic error and mitotic error. Meiotic error corresponds to the hypothesis S₂^{m} (2 different copies) and mitotic error corresponds to the hypothesis S₁^{m} (duplicate of the same homolog). The prior probabilities of these two events are assumed equal. As a result, a very high confidence on the S₁^{m} hypothesis puts approximately equal confidence on H1x and H2x, but a very high confidence on the S₂^{m} hypothesis favors only H2x.

This algorithm is well suited to detecting segmentation in chromosomes. A segmented disomy is characterized by the presence of a copy from each parent, where at least one parent's copy is incomplete. If one parent has greater than about 80 percent confidence on the 0 strands hypothesis (M₀ or F₀) for at least a quarter of the chromosome, this chromosome may be flagged as “segmented monosomy” even if the confidence calculations using other expert techniques result in a disomy call. This segmentation flag may be combined with the segmentation flag from the Permutation technique so that either one can independently detect an error. If the overall algorithm call is monosomy, the segmented flag may not be activated because it would be redundant.

At this point in the execution of the technique, copy hypothesis confidences have been assigned for each parent for each chromosome where dropout rates were available for at least one segment. However, some chromosomes may not have been phased with high confidence and their likelihoods may reflect dropout rates that were only available for a very small fraction of the chromosome. In one embodiment of the present disclosure, to avoid making calls based on insufficient or unclear data, checks may be performed to remove calls on chromosomes with incomplete phasing or very noisy results.

After the checks are performed, the parent copy hypotheses may be converted to the standard CNC hypotheses. For mother copies Nₘ and father copies N_{f}, the likelihood of the CNC hypothesis HNₘN_{f} is simply a multiplication of the independent parent copy likelihoods. If one parent was not called due to incomplete phasing or noisy data, the algorithm may output uniform likelihoods across that parent but still call the other parent.

P(D|HNₘN_{f})=P(D|HNₘx)P(D|HxN_{f})

### Check for Incomplete Phasing

The phasing coverage on a chromosome is the sum of segment lengths for which likelihoods were calculated. In some embodiments of the present disclosure, no likelihoods are calculated when any of the three dropout rate measurements has no data. If phasing coverage is less than half, no call is produced. In the case where meiotic trisomy is flagged by a sequence of M₁₂ or F₁₂ segments of combined length of about 0.25, any phasing coverage of less than 0.75 is not sufficient to rule out such a segment. However, if a meiotic segment of length 0.25 is detected, it may still be called. In one embodiment of the present disclosure, phasing coverage between about 0.5 and about 0.75 is dealt with as follows.

if it is flagged as trisomy, the ploidy call is as if completely phased

if the call is partial or complete monosomy, the ploidy call is as if completely phased

otherwise, do not call (set uniform likelihood for this parent's copies)

### Check for Noisy Chromosomes

Some chromosomes may resist classification using this algorithm. In spite of high confidence phasing and segment likelihoods, whole-chromosome results are unclear. In some cases, these chromosomes are characterized by frequent switching between maximum likelihood hypothesis. Although only a few recombination events are expected per chromosome, these chromosomes may show nearly random switching between hypotheses. Because the meiotic hypothesis is triggered by a meiotic sequence of length of about 0.25, false trisomies may often be triggered on noisy chromosomes.

In some embodiments of the present disclosure, the algorithm declares a “noisy chromosome” by combining adjacent segments with the same maximum likelihood hypothesis. The average length of these new segments is compared to the average length of the set of original segments. If this ratio is less than two, then few adjacent segments may have matching hypotheses, and the chromosome may be considered noisy. This test is based on the assumption that the original segmentation is expected to be somewhat uniform and dense. A switch to an optimal segmentation algorithm would require a new criterion.

If a chromosome is declared noisy for a particular parent, then the copy hypotheses for that parent may be set as uniform and the meiotic and segmented monosomy flags are set as false.

### Sex Chromosome Technique

The techniques described above are designed for autosomic chromosomes. Since the likely genetic states of the sex chromosomes (X and Y) are different, different techniques may be more appropriate. In this section several techniques are described that are designed specifically for determination of the ploidy state of the sex chromosomes.

In addition to the expected numbers of sex chromosomes being different, determination of the ploidy state of sex chromosomes is further complicated by the fact that there are regions on the X and Y chromosome that are homologous, and others that are similar but non-polymorphic. The Y chromosome may be considered to be a mosaic of different regions, and the behavior of the Y probes depends largely upon the region to which they bind on the Y chromosome. Many of the Y probes do not measure SNPs per se; instead, they bind to locations that are non-polymorphous on both the X and Y chromosomes. In some cases, a probe will bind to a location that is always AA on the X chromosome but always BB on the Y chromosome, or vice versa. These probes are termed “two-cluster” probes because when one of these probes is applied to a set of male and female samples, the resulting scatter plot always clusters into two clusters, segregated by sex. The males are always heterozygous, and the females are always homozygous.

### XYZ Chromosome Technique

In one embodiment of the present disclosure the ploidy determination of sex chromosomes is handled by considering an abstract chromosome termed “chromosome 23”, composed of four distinct sub-chromosomes, termed X, Y, XY, and Z. Chromosome XY corresponds to those probes that hybridize to both the X and Y chromosomes in what are known as the pseudoautosomal regions. In contrast, the probes associated with chromosome X are only expected to hybridize to chromosome X, and those probes associated with chromosome Y are only expected to hybridize to chromosome Y. Chromosome Z corresponds to those “two cluster” probes that hybridize to the Y chromosome in what is known as the X-transpose region the region that is about 99.9% concordant with a similar region on the X chromosome, and whose allele values are polar to their cognates in X. Thus, a Z probe will measure AB (disregarding noise) on a male sample, and either AA or BB on a female sample, depending on the locus.

The discussion below describes the math behind this technique. In terms of the component sex chromosomes, the goal of this technique is to distinguish the following cases: {0, X, Y, XX, XY, YY, XXX, XXY, XYY, XXYY}. Note that, if chromosome 23 is euploid, then it must be one of {XX,XY} and hence must have a copy number of 2. In the cases of uniparental disomy: XX from mother and nothing from father, or YY from father, one may arbitrarily assign the copy number of 5, or merge them in with the monosomy hypotheses.

The linkage between the X and Y sub-chromosomes expresses itself only in the joint prior distribution P(n_{X}^{F},n_{Y}^{F}) on the number of sub-chromosomes from X and Y contributed by the father.

### Notation

1. n is the chromosome copy number for chromosome 23.
2. n_{X}^{M} is the number of copies of sub-chromosome X supplied to the embryo by the mother: 0, 1, or 2. For notational purposes, it is convenient also to define n_{Y}^{M}=0 as the number of copies of sub-chromosome Y supplied to the embryo by the mother.
3. (n_{X}^{F},n_{Y}^{F}) are the number of copies of sub-chromosomes X and Y jointly supplied to the embryo by the father. These copy number pairs must belong to the set {(0,0),(0,1),(1,0),(2,0),(1,1),(0,2)}.
Note that the preceding three defined variables satisfy the constraint n_{X}^{M}+N_{X}^{F}+n_{Y}^{F}=n.
4. Define n_{XY}^{M}=n_{X}^{M}, n_{XY}^{F}=n_{X}^{F}+n_{Y}^{F}
5. Define n_{X}^{M}=n_{X}^{M}, n_{X}^{F}=n_{X}^{F}+n_{Y}^{F}
6. p_{d} is the dropout rate, and f(p_{d}) is a prior on this rate.
7. pₐ is dropin rate, and f(pₐ) is a prior on this rate.
8. c is the cutoff threshold for no-calls.
9. D_{X}={(x_{Xk},y_{Xk})} is the set of raw platform responses on channels x and y over all SNPs k on sub-chromosome X. Similarly D_{Y}={(x_{Yk},y_{Yk})} is the set of raw platform responses on channels x and y over all SNPs k on sub-chromosome Y, D_{XY}={(x_{XYk},y_{XYk})} is the set of raw platform responses on channels x and y over all SNPs k on sub-chromosome XY, and D_{Z}={(x_{Zk},y_{Zk})} is the set of raw platform responses on channels x and y over all SNPs k on sub-chromosome Z.
10. D_{X}(c)={G(x_{Xk},y_{Xk}),c}={ĝ_{Xk}^{(g)}} is the set of genotype calls over all SNPs k on sub-chromosome X, and similarly for sub-chromosomes Y, XY, and Z. Note that the genotype calls depend on the no-call cutoff threshold c.
11. Define a sub-chromosome index j, where jε{X,Y,XY,Z}. In this case, we can reference Dⱼ(c) to refer to the data associated with sub-chromosome j.
12. ĝⱼₖ^{(c)} is the genotype call on the k^{th} snp (as opposed to the true value) on sub-chromosome j: one of AA, AB, BB, or NC (no-call).
13. Given a genotype call ĝ at snp k, the variables (ĝ^{A},ĝ_{B}) are indicator variables (1 or 0). Formally, ĝ^{A}=(Aεĝ), and ĝ^{B}=(Bεĝ).
14. M−{g_{jY}^{M}} is the known true sequence of genotype calls on the mother on sub-chromosome j. g^{M} refers to the genotype value at some particular locus. Note that, for j=Y, {g_{Yk}^{M}} is taken to be a sequence of no-calls: NC.
15. F={gⱼₖ^{F}} is the known true sequence of genotype calls on the father on sub-chromosome j. g^{F} refers to the genotype value at some particular locus.
16. C_{MF}(f) is the class of conceivable joint parental genotypes that can occur on sub-chromosome j. Each element of C_{MF}(f) is a tuple of the form (G^{M},g^{F}), e.g., (AA, AB), and describes one of the possible joint genotypes for mother and father. The sets C_{MF}(j) are listed in full here:

a. C_{MF}(X)={AA,AB,BB}×{AA,BB}

b. C_{MF}(Y)={NC}×{AA,BB}

c. C_{MF}(XY)={AA,AB,BB}×{AA,AB,BB}

d. C_{MF}(Z)={AA,BB}×{AB}

17. nⱼ^{A},nⱼ^{B} are the true number of copies of A and B on the embryo (implicitly at locus k), respectively on sub-chromosome j. Values must be in 0, 1, 2, 3, 4 for jε{X,XY,Z} and in 0, 1, 2 for jε{Y}.
18cⱼ^{AM},cⱼ^{BM} are the number of A alleles and B alleles respectively supplied by the mother to the embryo (implicitly at locus k) on sub-chromosome j. For j=X or XY or Z, the values must be in 0, 1, 2, and must not sum to more than 2. For j=Y, the values must be (0,0). Similarly, cⱼ^{AF},cⱼ^{BF} are the number of A alleles and B alleles respectively supplied by the father to the embryo (implicitly at locus k) on sub-chromosome j. The father has the additional constraint for j=X or j=Y that one of cⱼ^{AF},cⱼ^{BF} must be zero, reflecting the fact that the father cannot contribute heterozygous material from either individual sex chromosome. For j=XY, there is no such constraint. For j=Z, the constraints are as follows:
1. When the locus is homo AA on the mother, then we have c_{z}^{AF}=n_{X}^{F} and c_{Z}^{BF}=n_{Y}^{F}.
2. When the locus is homo BB on the mother, then we have c_{Z}^{BF}=n_{X}^{F} and c_{Z}^{AF}=n_{Y}^{F}.

Altogether, the four values {cⱼ^{AM},cⱼ^{BM}cⱼ^{AF},cⱼ^{BM}} exactly determine the true genotype of the embryo on sub-chromosome j. For example, if the values were (1,1) and (1,0), then the embryo would have type AAB.

Note also that the following constraints hold for all j:

1. cⱼ^{AM}+cⱼ^{BM}=nⱼ^{M}
2. cⱼ^{AF}+c_{BF}=nⱼ^{F}

The following solution applies just to chromosome 23 and takes into account the interrelation between sub-chromosomes X,Y, and XY.

$$P ( x \vert {D}_{x} ( c ) , {D}_{Y} ( c ) , {D}_{XY} ( c ) , M , F ) = {\sum }_{\text{?}} P ( {n}_{x}^{M} , {n}_{x}^{F} , {n}_{x}^{F} [ {D}_{x} ( c ) , {D}_{Y} ( c ) , {D}_{XY} ( c ) , M , F ) \\ P ( {n}_{x}^{M} , {n}_{x}^{F} , {n}_{x}^{F} [ {D}_{x} ( c ) , {D}_{Y} ( c ) , {D}_{XY} ( c ) , M , F ) = \frac{P ( {n}_{x}^{M} ) P ( \text{?} ) P ( {D}_{X} ( c ) , {D}_{Y} ( c ) , {D}_{XY} ( c ) \vert \text{?} , M , F )}{\sum \text{?} P ( \text{?} ) P ( \text{?} ) P ( {D}_{X} ( c ) , {D}_{Y} ( c ) , {D}_{XY} ( c ) \vert {n}_{x}^{M} , {n}_{x}^{F} , {n}_{Y}^{F} , M , F )} \\ \text{?} \text{indicates text missing or illegible when filed}$$

P(n_{X}^{F},n_{Y}^{F}) is a prior distribution that may be set reasonably. The probabilities of (1,0) and (0,1) may be set reasonably high, as these are the euploidy states.

P(Dₓ(c),D_{Y}(c),D_{XY}(c)|n_{X}^{M},n_{X}^{F},M,F)=P(D_{X}(c)|n_{X}^{M},nX^{F},M,F)×P(D_{Y}(c)|n_{Y}^{F},M,F)×P(D_{XY}(c)|n_{XY}^{M},n_{XY}^{F},M,F)

Keep in mind in the above that n_{XY}^{M}=n_{X}^{M} and n_{XY}=n_{X}^{F}+n_{Y}^{F}.

P(Dⱼ(c)|nⱼ^{M},nⱼ^{F},M,F)=∫∫f(p_{d})f(pₐ)P(Dⱼ(c)|nⱼ^{M},nⱼ^{F},M,F,p_{d},pₐ)dp_{d}dpₐ

(*)P(Dⱼ(c)|nⱼ^{M},nⱼ^{F},M,F,p_{d},pₐ)=ΠₖP(G(xⱼₖ,yⱼₖ,c)|nⱼ^{M},nⱼ^{F},gⱼₖ^{M},gⱼₖ^{F},p_{d},pₐ)

### Handling (*) on XY Chromosome

The case of the XY chromosome behaves similarly to any autosome. The math is discussed here.

$$P ( x \vert {D}_{X} ( c ) \vert {n}_{x}^{M} , {n}_{X}^{F} , M , F , {p}_{d} , {p}_{a} ) = {{\prod }_{k}}_{\text{?}} P ( G ( {x}_{Xk} , {y}_{Xk} \vert c ) \vert {n}_{x}^{M} , {n}_{X}^{F} , {n}_{x}^{M} , {g}_{X}^{F} , {y}_{a} , {p}_{a} ) = {\prod }_{\text{?}} {\prod }_{\text{?}} \text{?} = {\prod }_{\text{?}} \text{?} = exp ( {\sum }_{\text{?}} \vert \{ \text{?} = \text{?} = \text{?} = \text{?} \} \vert log P ( \text{?} ) ) \text{?} = {\sum }_{\text{?}} \underbrace{\text{?}}_{genetic modeling} \overbrace{\text{?}}^{platform modeling}$$

$$\text{?} \text{indicates text missing or illegible when filed}$$

### Handling (*) on X Chromosome

The additional constraints here are that the father is never heterozygous on X.

$$P ( {D}_{X} ( c ) \vert \vert {n}_{x}^{M} , {n}_{X}^{F} , M , F , {p}_{d} , {p}_{a} ) = {{\prod }_{k}}_{\text{?}} P ( G ( {x}_{Xk} , {y}_{Xk} \vert c ) \vert {n}_{x}^{M} , {n}_{X}^{F} , {n}_{x}^{M} , {g}_{X}^{F} , {y}_{a} , {p}_{a} ) = {\prod }_{\text{?}} {\prod }_{\text{?}} \text{?} = {\prod }_{\text{?}} \text{?} = exp ( {\sum }_{\text{?}} \vert \{ \text{?} = \text{?} = \text{?} = \text{?} \} \vert log P ( \text{?} ) ) \text{?} = {\sum }_{\text{?}} {\sum }_{\text{?}} \underbrace{\text{?}}_{genetic modeling} \overbrace{\text{?}}^{platform modeling}$$

$$\text{?} \text{indicates text missing or illegible when filed}$$

### Handling (*) on Y Chromosome

The constraints here are that the mother's copy number is 0 and the father is never heterozygous on Y.

$$P ( {D}_{Y} ( c ) \vert \text{?} ) = {\prod }_{k} P ( \text{?} ) = {\prod }_{\text{?}} {\prod }_{\text{?}} P ( \text{?} ) = {\prod }_{\text{?}} P ( \text{?} ) = exp ( {\sum }_{\text{?}} \vert \{ \text{?} = \text{?} = \text{?} \} \vert \times log P ( \text{?} ) )$$

$$P ( g \vert n , {g}^{F} , {p}_{d} , {p}_{a} ) ≡ {\sum }_{} {P ( {n}^{A} , {n}^{B} \vert n , {g}^{F} )}_{\begin{matrix}⏟ \\ genetic modeling\end{matrix}} {P ( {g}^{A} \vert {n}^{A} , {p}_{d} , {p}_{a} ) P ( {g}^{B} \vert {n}^{B} , {p}_{d} , {p}_{a} )}^{\overbrace{platform modeling}} \\ P ( {n}^{A} , {n}^{B} \vert n , {g}^{F} , ) = P ( {n}^{A} , {n}^{B} \vert n , {g}^{F} , n = 0 , {g}^{M} = NC )$$

$$\text{?} \text{indicates text missing or illegible when filed}$$

Here the solution is continued for all sub-chromosomes. Keep in mind that when j=Y, then n_{J}^{M}=0 and gⱼₖ^{M}=NC for all k.

$$\begin{matrix}P ( g \vert {n}_{j}^{N} , \text{?} , {g}^{N} , {g}^{r} , \text{?} ) = {\sum }_{\text{?}} \underbrace{P ( {n}^{A} , {n}^{B} \vert {n}_{j}^{M} , {n}_{j}^{F} , {g}^{M} , {g}^{F} )}_{genetic modeling} \overbrace{P ( {g}^{A} \vert {n}^{A} , {p}_{a} , {p}_{a} ) P ( {g}^{B} \vert {n}^{B} , {p}_{a} , {p}_{a} )}^{platform modeling} & \end{matrix} P ( {g}^{d} \vert {n}^{d} , {p}_{d} , {p}_{a} ) = {g}^{d} ( ( 1 - {p}_{d}^{{n}^{A}} ) + ( {n}^{d} = 0 ) {p}_{a} ) + ( 1 - {g}^{d} ) ( ( {n}^{d} > 0 ) {p}_{d}^{{n}^{A}} + ( {n}^{d} = 0 ) ( 1 - {p}_{a} ) )$$

$$P ( {g}^{B} \vert {n}^{B} , {p}_{a} , {p}_{a} ) = {g}^{B} ( ( 1 - {p}_{a}^{{n}^{B}} ) + ( {n}^{B} = 0 ) {p}_{a} ) + ( 1 - {g}^{B} ( {n}^{B} > 0 ) {p}_{d}^{{n}^{B}} + ( {n}^{B} = 0 ) ( 1 - {p}_{a} ) )$$

$$P ( {n}^{d} , {n}^{B} \vert {n}_{j}^{M} , {n}_{j}^{F} , {g}^{M} , {g}^{F} , ) = {\sum }_{\text{?}} P ( {c}_{j}^{dM} , {c}_{j}^{BM} \vert {n}_{j}^{N} , {g}^{M} ) P ( {c}_{j}^{dF} , {c}_{j}^{BF} \vert {n}_{j}^{F} , {g}^{F} )$$

$$\text{?} \text{indicates text missing or illegible when filed}$$

Mother Sub-Cases: for j in {X, XY}, we have

$$P ( {c}_{j}^{dM} , {c}_{j}^{BM} \vert {n}_{j}^{M} , {g}^{M} ) = ( {c}_{j}^{dM} \vert {c}_{j}^{BM} = {n}_{j}^{M} ) \{ \begin{matrix}( {c}_{j}^{BM} = 0 ) , & {g}^{M} = AA \\ ( {c}_{j}^{dM} = 0 ) , & {g}^{M} = BB \\ \frac{1}{{n}_{j }^{M} + 1} , & {g}^{M} = AB\end{matrix}$$

For j=Y we have, which is degenerate for the mother, we have:

P(c_{Y}^{AM},c_{Y}^{BM}|n_{Y}^{M},g^{M})=(c_{Y}^{AM}+c_{y}^{B}=0)(n_{Y}^{M}=0)(g^{M}=NC)

Father Sub-Cases: for j in {X,Y}, we have:

$$P ( {c}_{j}^{AF} , {c}_{j}^{BF} \vert {n}_{j}^{F} , {g}^{M} ) = ( {c}_{j}^{AF} + {c}_{j}^{BF} = {n}_{j}^{F} ) ( ( {c}_{j}^{AF} = 0 ) ⋃ ( {c}_{j}^{BF} - 0 ) ) \{ \begin{matrix}( {c}_{j}^{BF} = 0 ) , & {g}^{F} = AA \\ ( {c}_{j}^{AF} = 0 ) , & {g}^{F} = BB\end{matrix}$$

For j=XY, the mathematics are the same as for the mother, viz:

$$P ( {c}_{XY}^{AF} , {c}_{XY}^{BF} \vert {n}_{XY}^{F} , {g}^{F} ) = ( {c}_{XY}^{dF} + {c}_{XY}^{EF} = {n}_{XY}^{F} ) \{ \begin{matrix}( {c}_{XY}^{BF} = 0 ) , & {g}^{F} = AA \\ ( {c}_{XY}^{dF} = 0 ) , & {g}^{F} = BB \\ \frac{1}{{n}_{XY}^{B} + 1} , & {g}^{F} = AB\end{matrix}$$

### X Chromosome Technique

In one embodiment of the present disclosure, the X-chromosome technique, described here, is able to determine the ploidy state of the X-chromosome with high confidence. In practice, this technique has similarities with the permutation technique, in that the determination is made by examining the characteristic CDF curves of the different contexts. This technique specifically uses the distance between certain context CDF curves to determine the copy number of the sex chromosome.

In one embodiment of the present disclosure, the algorithm may be modified in the following way to optimize for the X-chromosome. In this embodiment, slight modifications may be made in the allele distribution, the response model, and possible hypothesis. The formula is:

$$P ( {g}_{ij} \vert D , F ) = \frac{P ( \text{?} )}{P ( D \vert F )} {Σ}_{{g}^{M} , {g}^{F}} F ( {g}^{M} ) P ( {g}^{F} ) P ( {D}_{i}^{m} \vert {g}^{M} ) P ( {D}_{i}^{f} \vert {g}^{F} ) {Σ}_{n} P ( {g}_{ij} \vert {g}^{M} , {g}^{F} , h , F \text{?} ) * Q ( h , {g}^{M} , {g}^{F} , F , D , i , j ) where$$

$$Q ( h , {g}^{M} , {g}^{F} , F , D , i , j ) = {\sum }_{\text{?}} {\prod }_{\text{?}} P ( D \text{?} \vert {g}^{M} , {g}^{F} , H \text{?} , F \text{?} ) {\prod }_{\text{?}} P ( D \text{?} \vert {g}^{F} , H \text{?} , F \text{?} ) * {W}_{L} ( {H}_{i} , D , i , F ) * {W}_{2} ( {H}_{i} , D , i , F )$$

$$\text{?} \text{indicates text missing or illegible when filed}$$

In addition, some or all of the following changes may be made:

The response model P(D_{jf}^{e}|g_{jf},Fⱼ^{e}) depends on Fⱼ^{e}. If Fⱼ^{e}=0, 2 copies, then it may be modeled as before, if Fⱼ^{e}=1, use one copy and it may be modeled the same as for sperm.

P(g^{F}) is p, (1−p), for AA, BB respectively, omitting AB.

P(Dⱼ^{f}|g^{F}) is same as before, since we assume 100% correct parents, just make sure to omit any snips with Dⱼ^{f}=AB

h, the embryo hypothesis on (mother, father), previously had 4 possibilities, now only consider 2 possibilities for M1, M2, since contribution from the father either does not exist (for Fⱼ^{e}=0), or only has one hypothesis (for F_{f}^{e}1). This is valid for each embryo. Similarly on sperm there is only one hypothesis.

P(gᵢⱼ|g^{M},g^{F},h,Fⱼ^{e}) may be calculated slightly differently depending on Fⱼ^{e}, i.e depending on whether we consider the father's contribution.

Q(h,g^{M},g^{F},F,D,i,j) may be calculated the same way as before, taking into account the reduction in the hypothesis space, and above mentioned changes depending on Fⱼ^{e}.

### Context Distance: X Chromosome

In another embodiment of the present disclosure, the ploidy state of the X-chromosome may be determined as follows. The first step is to determine the distance between the following four contexts: AA|BB and BB|AA on channel X, AA|BB and BB|AA on channel Y, AB|BB and BB|AA on channel X, and AB|AA and AA|BB on channel Y. These distances may be taken at the point where AA|AA and BB|BB are furthest apart, and then normalized by the distance between AA|AA and BB|BB. This normalization serves as a way to remove any variation in the amplification process. Then distributions may be built for each of the normalized distances under the hypotheses H₁₀, H₀₁, H₁₁, H₂₁ and H₁₂ using high confidence ploidy calls on the autosomal chromosomes. In one embodiment of the present disclosure, the training set is restricted to chromosomes 1-15.

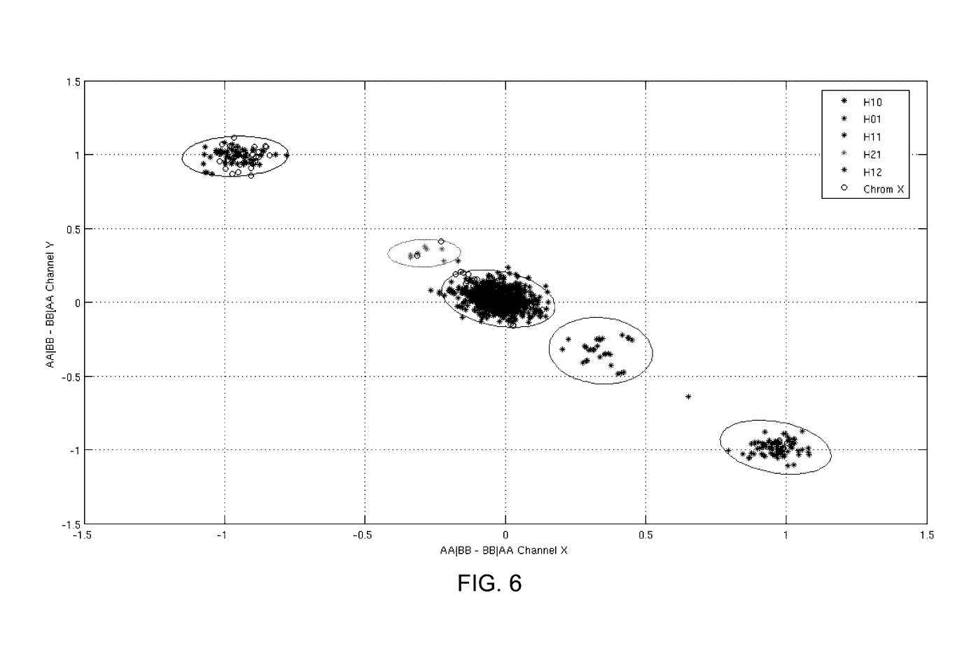

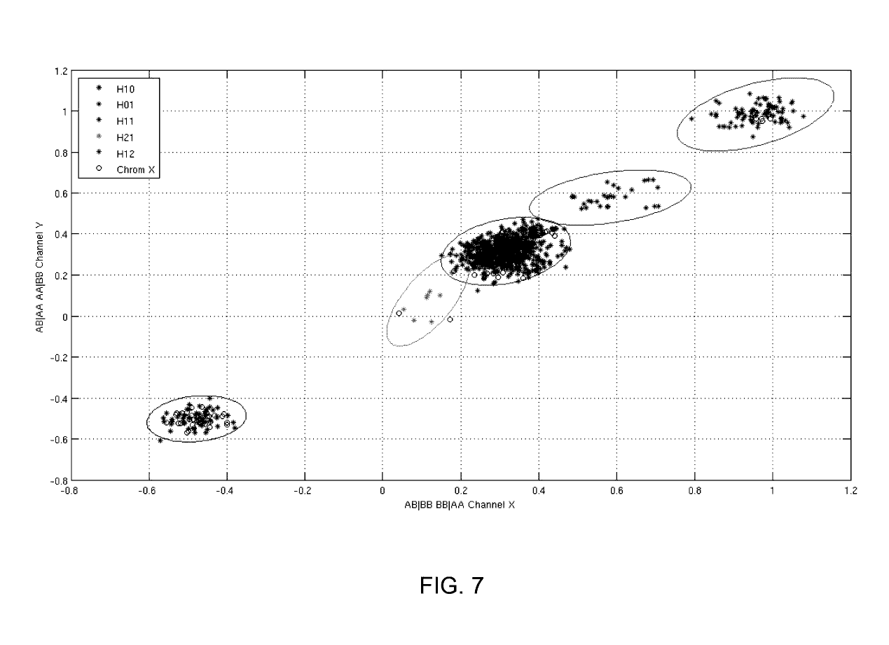

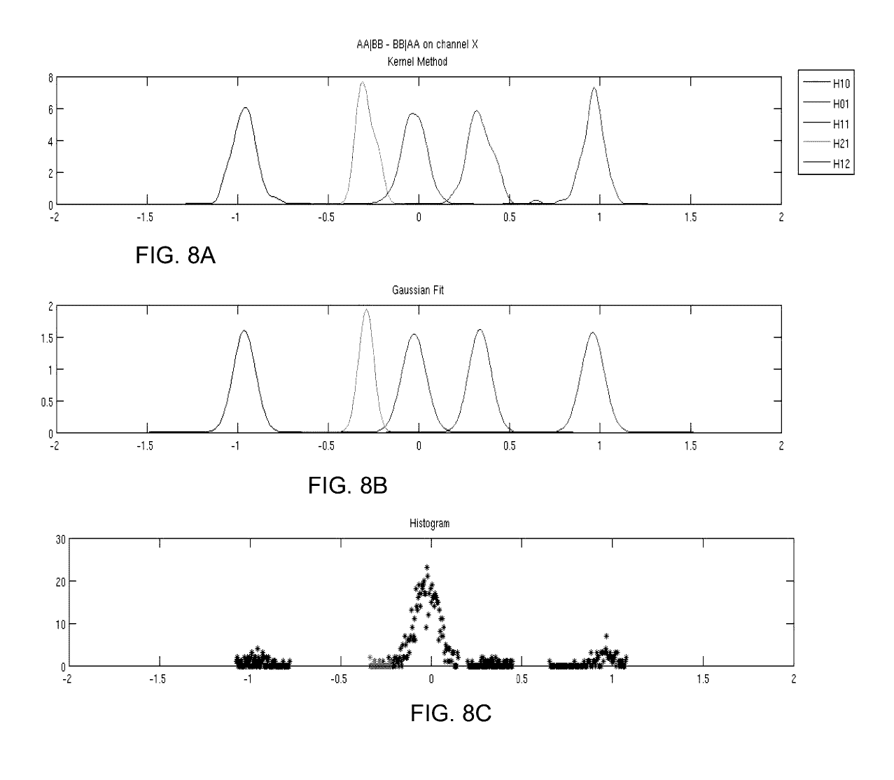

FIG. 6 and FIG. 7 present two graphs showing the clustering of the various contexts taken from actual data. FIG. 6 shows a plot of a first set of SNPs, with the normalized intensity of one channel output plotted against the other. FIG. 7 shows a plot of a second set of SNPs, with the normalized intensity of one channel output plotted against the other. The data presented in these two figures show that the data from the various contexts cluster well, and the hypotheses are clearly separable. Note that only chromosomes with confidence greater than about 0.9 were used for the training set. An example of the distribution of the distances can be seen in FIGS. 8A-8C, which show curve fits for allelic data for different ploidy hypotheses. FIG. 8A shows curve fits for allelic data for five different ploidy hypotheses using the Kernel method disclosed herein, FIG. 8B shows curve fits for allelic data for five different ploidy hypotheses using a Gaussian Fit disclosed herein, and FIG. 8C shows a histogram of the actual measured allelic data from one parental context, AA|BB−BB|AA, on channel X, as compared to the curve fits of all of the data. The ploidy state whose hypothesis best matches the actual measured allelic data is determined to be the actual ploidy state. This technique calls the ploidy state of the cell whose data is shown in FIGS. 6-8 as XX with confidence of about 0.999 or better. This method also made correct calls on single cells isolated from cell lines with known ploidy states.

### Y Chromosome

In one embodiment of the present disclosure, the ploidy state of the Y chromosome may be determined as described as elsewhere in this disclosure, with the following modifications. In one embodiment it is possible to use the presence of parents technique, with appropriate modifications for the Y chromosome.

Let F^{e}ⱼ=0, gᵢⱼ=NaN. For F^{e}ᵢ=1, gᵢⱼ=g^{F}, i.e. the same as father. In another embodiment, it is possible to take into account possible error in father measurement:

$$F ( {g}_{ij} \vert D , F ) = F ( {g}_{ij} ) F ( {D}_{i}^{f} \vert {g}_{ij} ) {\prod }_{\text{?}} F ( D \text{?} \vert {g}_{ij} , F \text{?} ) {\prod }_{\text{?}} F ( D \vert {g}_{ij} , F \text{?} )$$

$$\text{?} \text{indicates text missing or illegible when filed}$$

where P(gᵢⱼ) is the population frequency on this snip, P(Dᵢ^{j}|gᵢⱼ) is going to be 0/1. In one embodiment of the present disclosure, one may assume that there is no error on parents, in which case the Y chromosome algorithm is simple. In another embodiment, one may use an error model for the parents on Y chromosome, in which case P(Dᵢₐ^{e}|gᵢⱼ,Fₐ^{e}), which is either simple if Fₐ=0, or one may use an error model on the target, and on the Y chromosome.

### XY Chromosome

For the “XY” chromosome, it is possible to use the same algorithm as for other autosomal chromosomes.

### Z Chromosome

In One embodiment, the “Z” chromosome has been defined such that the alleles must be AB for males and AA/BB for females, determined by population frequency. In this embodiment, one may make the following modifications:

$${g}_{ij} = \{ \begin{matrix}AB & F \text{?} = 1 \\ AA & F \text{?} = 0 , p ( A ) = 1 \\ BB & F \text{?} = 0 , p ( A ) = 0\end{matrix} \\ \text{?} \text{indicates text missing or illegible when filed}$$

In other respects the determination of the ploidy state of the Z chromosome may be done as described elsewhere in this disclosure.

### Non-Parametric Technique

In another embodiment of the present disclosure, an approach termed the “non-parametric technique” may be used. This technique makes no assumptions on the distribution of the data. For a given set of SNPs, typically defined by a parental context, it builds the expected distribution based on hypothetical or empirical. The determination of the probabilities of the hypotheses is made by comparing the relationship between the observed distributions of the parental contexts to expected relationships between the distributions of the parental contexts. In one embodiment, the means, quartiles or quintiles of the observed distributions may be used to represent the distributions mathematically. In one embodiment, the expected relationships may be predicted using theoretical simulations, or they may be predicted by looking at empirical data from known sets of relationships for chromosomes with know ploidy states. In one embodiment, the theoretical distributions for a given parental context may be constructed by mixing the observed distributions from other parental contexts. The expected distributions for parental contexts under different hypotheses may be compared to the observed distributions of parental contexts, and only the distribution under the correct hypotheses is expected to match the observed distribution.

Outline in this section is a method for computing posterior probabilities such as P(Hᵢ|“data”) where Hᵢ is a hypothesis that is some combination of the expected sets of distributions for cases where a parent contributes 0, 1, or 2 chromosomes. For the cases where the parent contributes two chromosomes, there are two possible sub-cases: M1 copy error (unmatched copy error) (2a), or M₂ copy error (matched copy error) (2b). This gives rise to 16 total hypotheses: four hypotheses for the father, multiplied by four for the mother. The case where either the mother or the father contributes at least one chromosome will be discussed first, and the case where a parent contributes no chromosomes will be discussed afterwards. Consider the following points:

(A) Under the parental contexts AB|AA and AA|AB, under the 8 parental chromosome contribution hypotheses where each parent contributes at least one chromosome, but not including the case where both parents contributed two chromosomes due to an M2 copy error, the distribution of the target genotypes can be separated into a distribution which can be computed empirically from the data. Furthermore, the distribution from the euploid state can be separated from the other hypotheses.
(B) If the distributions of the targets are different, there is a statistic T (formally here a random variable) that distinguishes them. The distribution of this statistic can be simulated by bootstrapping the distribution of the target under the parental contexts AB|AA and AA|AB. This produces an empirical p value under each hypothesis. The empirical p value for under the hypothesis will be denoted 0 and is defined as

\hat{p}ᵢ=P(T≧t|hypothesis t) (1)

where T is the random variable and we see a realization of the statistic t. The distribution of T under hypothesis i may be simulated with the bootstrap.

Empirical p values will produce posterior distributions of P(Hᵢ|“data”) via formalizing “data” as the event (a random variable) 1_{T≧t} with T defined on the joint probability space including all hypotheses and their sub hypotheses. This makes the above equation equivalent to P(Hᵢ|1_{T≧t}) which by Bayes' gives

$$P ( {H}_{i} \vert {1}_{T \geq t} \vert {H}_{1} ) \frac{P ( {H}_{i} )}{P ( {1}_{T \geq t} )} = {p}_{i} \frac{{p}_{{H}_{i}}}{p ( {1}_{T \geq t} )}$$

where \hat{p}ᵢ as in Equation so that P(1_{T≧t})=Σᵢ\hat{p},p_{Hi} and p_{Hi} is the prior on hypothesis i.

Denote (1,2a) as the case where the mother contributes 1 chromosome and the father contributes 2 under an m1 copy error. For the purpose of this discussion, assume an M1 copy error on a heterozygous locus implies AA, AB, and BB each occur with probability ⅓. In an M2 copy error, one chromosome is duplicated, so for a heterozygous locus, assume that AA and BB are seen each with probability ½.

Point (A) may be shown by investigating the distribution of the target under the different hypotheses. Note that (1,1) is the only case where F₁=F₂ and both are mixtures of two different distributions. These may be simulated using polar and non-polar homozygous SNPs). This is a good technique for identifying trisomy, but it is difficult to calculate a confidence for because it is difficult to simulate its distribution. For example, consider the median statistic T=medtan_{AABB}{zᵢ^{X}−zᵢ^{Y}}−medtan_{BBAA}{wᵢ^{X}−wᵢ^{Y}}, which is good algorithmically at separating (1,1) from (2a/b,1) or (1,2a/b). Again, there is not a confidence associated, because its distribution under the hypothesis of (1,2a/b) is simulated in the same way as (1,1), namely, if there are cases of AA|BB and n₂ cases of BB|AA, the simulated distribution is a mixture distribution of AA|BB and BB|AA resampled with proportions n₁/(n₁+n₂) and n₂/(n₁+n₂). Thus, T compared to its simulated distribution under trisomy will be expected to be the same as T compared to its simulated distribution under euploid. The explanation below describes how to overcome this problem, with the unlikely exception of the case where each parent donates two copies of a given chromosome to the embryo.

In the explanation here, F₁ denotes the distribution of the target loci under the parental context AB|AA and F₂ the distribution of the target loci under the parental context AA|AB.

1. (1,1): the distributions F₁=F₂ and is a mixture of ½ AA and ½ AB
2. (2b, 1): F₁ is a mixture of ½ AAA and ½ BBA. F₂ is a mixture of ½ AAA and ½ AAB.
3. (2a, 1): F₁ is a mixture of AAA ABA and BBA. I will be assuming the mixture is for each although that may not be necessary for the method. F₂ is equal to a mixture of ½ AAA and ½ AAB.
4. (1,2b) F₁ is the same as F₂ in item 2 by symmetry and F₂ is the same as F₁ in item 2 by symmetry.
5. (1,2a) F₁ is the same as F₂ in item 3 by symmetry and F₂ is the same as F₁ in item 3 by symmetry.
6. (2a, 2b) F₁ is a mixture of ⅓ each of AAAA ABAA BBAA, F₂ is a mixture of ½ of AAAA AABB.
7. (2b, 2a) both Fᵢ is F₂ of the previous item and F₂ is F₁ of the previous item by symmetry.
8. (2a, 2a) F₁ is a mixture of ⅓ each of AAAA ABAA BBAA, F₂ equals F₁.
9. (2b, 2b) F₁ is a mixture of ½ AAAA, ½ BBAA. F₂ has the same distribution as F₁.

The algorithmic approach is as follows:
Find a good statistic F₁ of target channels under parent context AA|AB and a good statistic F₂ of target channels under parent context AB|AA. In one embodiment, let t₁ and t₂ be the means of zᵢ^{x}−zⱼ^{y} under AA|AB and AB|AA, respectively.)
Under hypothesis i, produce empirical joint null distributions (\hat{F}₁,\hat{F}₂) using a mixture of resampled data from polar homozygotes when possible, this is usually possible; otherwise use resampling of heterozygots.
Compare the joint distribution of (t₁,t₂) to the empirical, which produces the empirical p value.
Compute the empirical p value as described in the first part of the document.
Classify according to maximum posterior probability and assign posterior probability to the call.
To increase the power of this procedure, one may include distributions F₃,F₄ which correspond to F₁ and F₂ but interchange the alleles A and B.

Now consider the cases where one parent contributes no chromosomes:
1. (0,0): F₁ and F₂ are noise, these could be simulated using any SNPs. In one embodiment, one could use the context AA|AA and BB|BB.
2. (0,1): F₁ is a ½ mixture of A and B F₂ is A
3. (0, 2a): F₁ is AA and F₂ is BB.
4. (0, 2b): F₁ is AA and F₂ is a mixture of AA AB BB.
5. (1,0) switch F₁ and F₂ from the case of (0,1) by symmetry.
6. (2a, 0) switch F₁ and F₂ from the case of (0,2a) by symmetry.
7. (2b, 0) switch F₁ and F₂ from the case of (0,2b) by symmetry.

### Confidence Sketch for Non-Parametric Technique

The analysis of the algorithm is based on the idea that for the i^{th} hypothesis, Hᵢ, one may to compute the probability that some (another or the same) hypothesis Hⱼ is true given the data P(Hᵢ|data), which is equivalent to P(“algorithm calls”Hᵢ|data).

Using priors, one may compute P(data|Hᵢ). In one embodiment, the algorithm may be simplified by using parental context 1. In another embodiment, all three contexts may be used. Therefore, one may write the analysis for the algorithm that calls euploid just when

$$\frac{\vert \text{?} \vert}{\text{?}}$$

$$\text{?} \text{indicates text missing or illegible when filed}$$

is smaller than a threshold t where \hat{p}_{g} is the re-estimate of q using only parental context 1 which is the polar homozygotes. Also, note the algorithm is calling ploidy state based on a modified thresholding scheme where the re-estimate \hat{p}_{q} is compared to q and normalized based on the estimated standard error of αₚ_{_{q}}. The algorithm works on autosomes and sex chromosomes in this way.

Fix a particular context and assume the Zᵢ and Wⱼ have the following distribution:

Zᵢ=μ_{Z}+σᵢ²εᵢ and

Wⱼ=μ_{W}+σⱼ²εⱼ (1)

where the εᵢ and εⱼ are assumed I.I.D., and {σᵢ}ᵢ₌₁ⁿ are constants. In practice, z₁, . . . zₙ_{_{Z}} and w₁, . . . wₙ_{_{Z}} are observed, realizations of the random variables {Zᵢ}ᵢ₌₁ⁿ^{ₛ} and {Wᵢ}ᵢ₌₁ⁿ^{_{w}}.

To analyze the quantile calling algorithm, assume the q^{th} quantile of ε equals 0. This is without loss of generality because, for example, quantile calling is invariant under multiplicative scaling of the Zᵢ and Wⱼ and adding a constant to all Zᵢ and Wⱼ.

Assume all σᵢ² are equal to simplify and let z_{q} be the q^{th} quantile of the Zᵢ. Define/denote the p_{q} by

p_{q}=P(Wⱼ<z_{q}).

Then, under the euploid condition, since μ_{Z}=μ_{W}, for each εᵢ,

p_{q}=P(μ_{W}+εᵢ<μ_{Z})=q,

where the

P(μ_{W}+εᵢ<μ_{Z})−E(1{μ_{W}+εᵢ<μ_{Z}})

### Outline of Probability Calculations

To understand the broad idea, consider a simplified case: suppose that the σᵢ are all the same and z_{q} are known exactly. Then, the estimator of p_{q}, denoted \hat{p}_{q} which in general is

$${\hat{p}}_{q} = \frac{1}{n \text{?}} {Σ}_{i = 1}^{{n}_{ij}} 1 \text{?}$$

$$\text{?} \text{indicates text missing or illegible when filed}$$

would be simplified to

$${p}_{q} = \frac{1}{{n}_{ij}} {Σ}_{i = 1}^{{n}_{ij}} 1 \text{?} , \\ \text{?} \text{indicates text missing or illegible when filed}$$

In this case, Wᵢ are i.i.d., z_{q} is known and hence \hat{p}_{q} is simply a mean of I.I.D. Bernouillis. This is an estimator which is simpler. The central limit theorem, which may be used to get exact information about the quality of approximation, says that

$$\frac{\text{?}}{{v}_{jq}} \\ \text{?} \text{indicates text missing or illegible when filed} \qquad ( 2 )$$

has an approximate normal distribution.

This method may be used to get confidences because under euploidy, p_{q}=q and under aneuploidy, if it is assumed that under the j^{th} type aneuploidy there is a difference δⱼ between p_{q} and q, (j=1 means parental contributions (0,0), j=1 means parental contributions (1,0), . . . p_{q}−μ>δⱼ. In one embodiment, the estimate of δ may be between 0 and 0.5.

Now assume, for simplicity, that all hypotheses are collapsed into M_{q}, the hypothesis of euploidy and M_{δ} the hypothesis of aneuploidy and denote δ as the smallest δⱼ

Define

$${\hat{z}}_{1} = \frac{\text{?}}{\text{?}} \\ \text{?} \text{indicates text missing or illegible when filed} \qquad ( 3 )$$

where \hat{z}_{Fq} is some estimate of σ_{Fq}, by bootstrap, or by the Bernoulli variance formula. The algorithm sets some threshold t and calls M_{δ} iff |\hat{z}|<t. Therefore, under euploidy, using the normal approximation, 2 has an approximate standard normal distribution so

P(Hₑ called|euploid condition)=P(|\hat{z}|<t)≅P(|N(0,1)|<t)≅0.99 for t=3.

For t=3, this probability is approximately 0.99. Therefore:

P(Hₐ called|euploid condition)≅0.01.

Conversely, under aneuploidy, \hat{z} has a normal distribution with mean

$$\frac{\text{?}}{\text{?}}$$

$$\text{?} \text{indicates text missing or illegible when filed}$$

and a variance of 1. Typically, σ_{Fq} is in the range of 0.01, therefore, of δ=(0.01)c for a constant c. In some embodiments c may be between about 1 and about 10, and another embodiment, c may be between about 10 and about 100.

P(Hₐ called|aneuploid condition)=P(|\hat{z}|<t)≅P(|N(3,1)|<t)

is small. For t=3, this probability is approximately (1−0.98)/2. Therefore,

P(Hₐ called|aneuploid condition)≅1−0.01.

Other possible expert techniques that may be used in the context of ploidy calling, and the list described in this disclosure is not meant to be exhaustive. Some further techniques are outlined below.

### Allele Calling

In the context of PGD during IVF, there is a great need to determine the genome of the embryo. However, genotyping a single cell often results in a high rate of allele drop out, where many alleles give an incorrect or no reading. Accurate genetic data of the embryo is required to detect disease-linked genes with high confidence, and those determinations may then be used to select the best embryo for implantation. One embodiment of the present disclosure, described herein, involves inferring the genetic data of an embryo as accurately as possible. The obtained data may include the measured genetic data, across the same set of n SNPs, from a target individual, the father of the individual, and the mother of the individual. In one embodiment, the target individual may be an embryo. In one embodiment, the measured genetic data from one or more sperm from the father are also used. In one embodiment, the measured genetic data from one or more siblings of the target individual are also used. In one embodiment, the one or more siblings may also be considered target individuals. One way to increase the fidelity of allele calls in the genetic data of a target individual for the purposes of making clinically actionable predictions is described here. Note that the method may be modified to optimize for other contexts, such as where the target individual is not an embryo, where genetic data from only one parent is available, where neither, one or both of the parental haplotypes are known, or where genetic data from other related individuals is known and can be incorporated.

The present disclosures described in this and other sections in this document have the purpose of increasing the accuracy of the allele call at alleles of interest for a given number of SNPs, or alternately, decreasing the number of SNPs needed, and thus the cost, to achieve a given average level of accuracy for SNP calls. From these allele calls, especially those at disease linked or other phenotype linked genes, predictions can be made as to potential phenotypes. This information can be used to select (an) embryo(s) with desirable qualities for implantation. Since PGD is quite expensive, any novel technology or improvement in the PS algorithms, that allows the computation of the target genotype to be achieved at a given level of accuracy with less computing power, or fewer SNPs measured, will be a significant improvement over prior technology.

This disclosure demonstrates a number of novel methods that use measured parental and target genetic data, and in some cases sibling genetic data, to call alleles with a high degree of accuracy, where the sibling data may originate from born siblings, or other blastomeres, and where the target is a single cell. The method disclosed shows the reduction to practice, for the first time, of a method capable of accepting, as input, uncleaned genetic data measured from a plurality of related individuals, and also determining the most likely genetic state of each of the related individuals. In one embodiment, this may mean determining the identity of a plurality of alleles, as well as phasing any unordered data, while taking into account crossovers, and also the fact that all input data may contain errors.

Genetic data of a target can be described given the measured genetic data of the target, and of the parents of the target, where the genetic data of the parents is assumed to be correct. However, all measured genetic data is likely to contain errors, and any a priori assumptions are likely to introduce biases and inaccuracies to the data. The method described herein shows how to determine the most likely genetic state for a set of related individuals where none of the genetic data is assumed to be true. The method disclosed herein allows the identity of each piece of measured genetic data to be influenced by the measured genetic data from each of the other related individuals. Thus, incorrectly measured parental data may be corrected if the statistical evidence indicates that it is incorrect.

In cases where the genetic data of an individual, or a set of related individuals, contains a significant amount of noise, or errors, the method disclosed herein makes use of the expected similarities between genetic data of those related individuals, and the information contained in the genetic data, to clean the noise in the target genome, along with errors that may be in the genetic data of the related individuals. This is done by determining which segments of chromosomes were involved in gamete formation and where crossovers occurred during meiosis, and therefore which segments of the genomes of related individuals are expected to be nearly identical to sections of the target genome. In certain situations this method can be used to clean noisy base pair measurements, but it also can be used to infer the identity of individual base pairs or whole regions of DNA that were not measured. In an embodiment, unordered genetic data may be used as input, for the target individual, and/or for one or more of the related individuals, and the output will contain the phased, cleaned genetic data for all of the individuals. In addition, a confidence can be computed for each reconstruction call made. Discussions concerning creating hypotheses, calculating the probabilities of the various hypotheses, and using those calculations to determine the most likely genetic state of the individual can be found elsewhere in this disclosure.

A highly simplified explanation of allele calling is presented first, making unrealistic assumptions in order to illustrate the concept of the present disclosure. A detailed statistical approach that can be applied to the technology of today is presented afterward.

### A Simplified Example

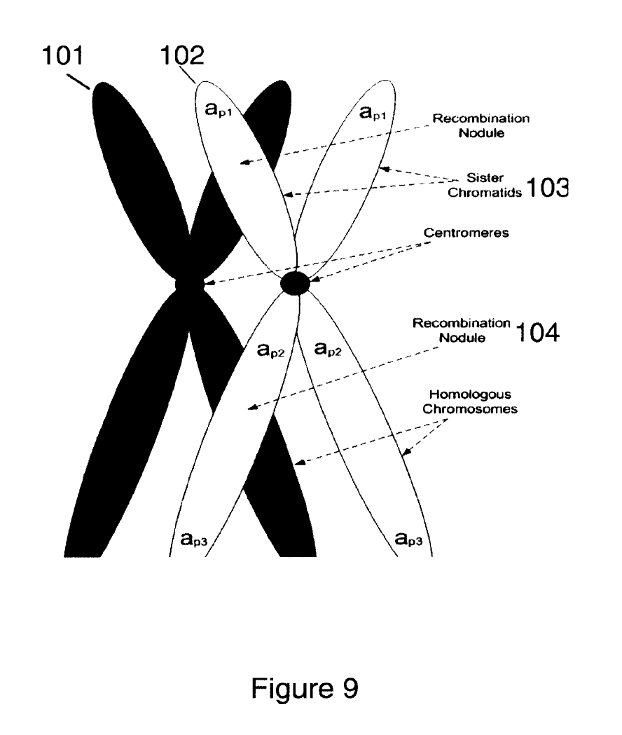

FIG. 9 illustrates the process of recombination that occurs during meiosis for the formation of gametes in a parent. The chromosome 101 from the individual's mother is shown in grey. The chromosome 102 from the individual's father is shown in white. During this interval, known as Diplotene, during Prophase I of Meiosis, a tetrad of four chromatids 103 is visible. Crossing over between non-sister chromatids of a homologous pair occurs at the points known as recombination nodules 104. For the purpose of illustration, the example will focus on a single chromosome, and three SNPs, which are assumed to characterize the alleles of three genes. For this discussion it is assumed that the SNPs may be measured separately on the maternal and paternal chromosomes. This concept can be applied to many SNPs, many alleles characterized by multiple SNPs, many chromosomes, and to the current genotyping technology where the maternal and paternal chromosomes cannot be individually isolated before genotyping.

Attention should be paid to the points of potential crossing over in between the SNPs of interest. The set of alleles of the three maternal genes may be described as (aₘ₁, aₘ₂, aₘ₃) corresponding to SNPs (SNP₁, SNP₂, SNP₃). The set of alleles of the three paternal genes may be described as (aₚ₁, aₚ₂, aₚ₃). Consider the recombination nodules formed in FIG. 1, and assume that there is just one recombination for each pair of recombining chromatids. The set of gametes that are formed in this process will have gene alleles: (aₘ₁, aₘ₂, aₚ₃), (aₘ₁, aₚ₂, aₚ₃), (aₚ₁, aₘ₂, aₘ₃), (aₚ₁, aₚ₂, aₘ₃). In the case with no crossing over of chromatids, the gametes will have alleles (aₘ₁, aₘ₂, aₘ₃), (aₚ₁, aₚ₂, aₚ₃). In the case with two points of crossing over in the relevant regions, the gametes will have alleles (aₘ₁, aₚ₂, aₘ₃), (aₚ₁, aₘ₂, aₚ₃). These eight different combinations of alleles will be referred to as the hypothesis set of alleles, for that particular parent.

The measurement of the alleles from the embryonic DNA is typically noisy. For the purpose of this discussion take a single chromosome from the embryonic DNA, and assume that it came from the parent whose meiosis is illustrated in FIG. 9. The measurements of the alleles on this chromosome can be described in terms of a vector of indicator variables: A=[A₁ A₂ A₃]^{T} where A₁=1 if the measured allele in the embryonic chromosome is aₘ₁, A₁=−1 if the measured allele in the embryonic chromosome is aₚ₁, and A₁=0 if the measured allele is neither aₘ₁ or aₚ₁. Based on the hypothesis set of alleles for the assumed parent, a set of eight vectors may be created which correspond to all the possible gametes describe above. For the alleles described above, these vectors would be a₁=[1 1 1]^{T}, a₂=[1 1 −1]^{T}, a₃=[1 −1 1]^{T}, a₄=[1 −1 −1]^{T}, a₅=[−1 1 1]^{T}, a₆=[−1 1 −1]^{T}, a₇=[−1 −1 1]^{T}, a₈=[−1 −1 −1]^{T} In this highly simplified application of the system, the likely alleles of the embryo can be determined by performing a simple correlation analysis between the hypothesis set and the measured vectors:

i*=arg maxᵢA^{T}aᵢ, i=1 . . . 8

Once i* is found, the hypothesis aᵢ* is selected as the most likely set of alleles in the embryonic DNA. This process may be repeated twice, with two different assumptions, namely that the embryonic chromosome came from the mother or the father. That assumption which yields the largest correlation A^{T}aᵢ* would be assumed to be correct. In each case a hypothesis set of alleles is used, based on the measurements of the respective DNA of the mother or the father.

Note that in one embodiment, those SNPs that are important due to their association with particular disease phenotypes may be referred to these as Phenotype-associated SNPs or PSNPs. In this embodiment, one may measure a large number of SNPs between the PSNPs, termed non-phenotype-associated SNPs (NSNPs), that are chosen a-priori (for example, for developing a specialized genotyping array) by selecting from the NCBI dbSNP database those RefSNPs that tend to differ substantially between individuals. Alternatively, the NSNPs between the PSNPs may be chosen for a particular pair of parents because the alleles for the parents are dissimilar. The use of the additional SNPs between the PSNPs enables one to determine with a higher level of confidence whether crossover occurs between the PSNPs. It is important to note that while different “alleles” are referred to in this notation, this is merely a convenience; the SNPs may not be associated with genes that encode proteins.

### A More Thorough Treatment of the Allele Calling Method

In the simplified example given above, for the purpose of illustration of the concept, the assumption is made that the parental genotypes are phased and known correctly. However, in many cases, this assumption may not hold. For example, in the context of genotyping of embryos during IVF, typically the measured genetic data from the parents are uncleaned and unphased, any measured genetic data from sperm from the father are uncleaned, and the measured genetic data from one or more blastomeres, biopsied from one or more embryos are also are uncleaned and unphased. In theory, the knowledge of the uncleaned, unphased embryo derived genetic data can be used to phase and clean the parental genetic data. In addition, in theory the knowledge of the genotype of one embryo can be used to help clean and phase the genetic data of another embryo. In some cases, the measured genetic data of several sibling target individual may be correct at a given set of alleles, while the genetic data of a parent may be incorrect at those same alleles. In theory, the knowledge of the target individuals could be used to clean the data of the parent.

In some embodiments of the present disclosure disclosed herein, methods are described which allow the parental genetic data to be cleaned and phased using the knowledge of the genetic data of the target and other related individuals. In some embodiments, methods are described which allow the genetic data to be cleaned and phased also using the knowledge of the genetic data of sibling individuals. In an embodiment of the present disclosure, the genetic data of the parents, of the target individual, and of one or a plurality or related individuals, is used as input, where each piece of genetic data is associated with a confidence, and the knowledge of the expected similarities between all of the genotypes is used by an algorithm that selects the most likely genetic state of all of the related individuals, at once. The output of this algorithm, the most likely genetic state of the related individuals, may include the phased, cleaned genetic allele call data. In some embodiments of the present disclosure, there may be a plurality of target individuals, and these target individuals may be sibling embryos. In some embodiments of the present disclosure, the methods disclosed in the following section may be used to determine the statistical probability for an allelic hypothesis given the appropriate genetic data.

In some embodiments of the present disclosure, the target cell is a blastomere biopsied from an embryo in the context of preimplantation diagnosis (PGD) during in vitro fertilization (IVF). In some embodiments, the target cell may be a fetal cell, or extracellular fetal DNA in the context of non-invasive prenatal diagnosis. Note that this method may apply to situations in other contexts equally well. In some embodiments of the present disclosure, a computational device, such as a computer, is leveraged to execute any calculations that make up the method. In one embodiment of the present disclosure, the method disclosed herein uses genetic data from the target individual, from the parents of the target individual, and possibly from one or more sperm, and one or more sibling cells to recreate, with high accuracy, the genomic data on the embryo while accurately taking into account crossovers. In one embodiment of the present disclosure, the method may be used to recreate genetic data for target individuals at aneuploid, as well as euploid chromosomes. In one embodiment of the present disclosure, a method is described for determining the haplotypes of parent cells, given diploid parent data and diploid genetic data from one or more blastomeres or other sibling cells, and possibly, but not necessarily, one or more sperm cells from the father.

### Practical Description of Allele Calling

In the following section, a description is given for a method for determining the genetic state of one or a series of target individuals. The description is made in the context of embryo genotype determination in the context of an IVF cycle, but it is important to note that the method described herein is equally well applicable other contexts, for other sets of related individuals, for example, in the context of non-invasive prenatal diagnosis, when the target individual is a fetus.

In the context of an IVF cycle, for a particular chromosome, the genotyping technique outputs data for n SNP locations, for k distinct targets (embryos or children) is made available by the genotyping technique. Each of the targets may have genotypes measured for one or more samples, and the measurements may be made on amplifications from a single cell, or from a small number of cells. For each SNP, each sample measurement consists of (X,Y) channel response (intensity) measurements. The X channel measures the strength of one (A) allele, and the Y channel measures the strength of the other (B) allele. If the measurements were completely accurate, on a particular SNP, an allele that is AA should have normalized (X,Y) intensities (arbitrary units are used) of (100,0), an allele that is AB should have intensities of (50,50) and an allele that is BB should have intensities of (0,100), and in this ideal case, it would be possible to derive exact allele values given the (X,Y) channel intensities. However, target single cell measurements are typically far from ideal, and it is not possible to determine, with high confidence, the true allele value given the raw channel responses.

Allele calling may be done for each chromosome separately. This discussion focuses on one particular autosomal chromosome with n SNPs. The first step is to define the nomenclature of the input data. The input data for the algorithm may be the uncleaned, unordered output data from genotyping array assays, it may be sequence data, it may be partially or fully processed genotype data, it may be known genotype data of an individual, or it may be any type of genetic data. The data may be arranged into target data, parental data, and sperm gametes, but this is not necessary. In the context of IVF, the target data would refer to genetic data measured from blastomeres biopsied from embryos, and it may also refer to genetic data measured from born siblings. The sperm data could refer to any data measured from a single set of chromosomes derived from a parent including sperm, polar bodies, unfertilized eggs or some other source of monosomic genetic matter. The data is arranged into various categories here for ease of understanding, but this is not necessary.

In this disclosure, the input data is labeled as follows: D refers to a set of genetic data from an individual. D^{T}=(D^{T1}, . . . , D^{Tk}) refers to the genetic data from k distinct targets (embryos/children), D^{S}=(D^{S1}, . . . , D^{Sl}) refers to the data from l distinct sperms, (D^{M}) refers to the data from the mother, and (D^{F}) refers to the data from the father. One may write D=(D^{T}, D^{S}, D^{M}, D^{F}). Written differently, by SNPs, where the subscript i refers to the i^{th} SNP in the set of data, D=(D₁, . . . , Dₙ), where Dᵢ=(D^{T}ᵢ, D^{S}ᵢ, D^{M}ᵢ, D^{F}ᵢ).

For k distinct targets, one may write D^{T}ᵢ (D^{T1}ᵢ, D^{T2}ᵢ, . . . D^{Tk}ᵢ). Each distinct target may have multiple resamples; a resample refers to an additional genotype reading made from a given sample. For the j^{th} distinct target one may write D^{Tj}ᵢ (D^{Tj,1}ᵢ, D^{Tj,2}ᵢ, . . . D^{Tj,kj}ᵢ) where kj=number of samples for target j. For r^{th} resample of target j on SNP i, one will observe the set of channel intensities D^{Tj,r}ᵢ=(X^{Tj,r}ᵢ, Y^{Tj,r}ᵢ).

A plurality of sperm may be considered, and on SNP i one may write D^{S}ᵢ=(D_{S1}ⁱ, D^{S2}ᵢ, . . . , D^{Sl}ᵢ) for l distinct targets. Each distinct sperm may also have multiple resamples. Thus for r^{th} distinct sperm D^{Sj}ᵢ=(D^{Sj,1}ᵢ, D^{Sj,2}ᵢ, . . . , D^{Sj,lj}ᵢ) where lj=number of resamples for sperm j. For the r^{th} resample of sperm j on SNP i, one will observe the set of channel intensities D^{Sj,r}ᵢ=(X^{Sj,r}ᵢ, Y^{Sj,r}ᵢ).

The genetic data of the mother, on SNP i, is D^{M}ᵢ=(D^{M,1}ᵢ, D^{M,2}ᵢ, . . . , D^{M,a}ᵢ). The genetic data of the mother may also have multiple resamples, and for the r^{th} resample of the mother on SNP i, one will observe the set of channel intensities D^{M,r}ᵢ=(X^{M,r}ᵢ, Y^{M,r}ᵢ).

The genetic data of the father, on SNP i, is D^{F}ᵢ=(D^{F,1}₁, D^{F,2}ᵢ, . . . D^{F,b}ᵢ) The genetic data of the father may also have multiple resamples, and for the r^{th} resample of the father on SNP i, one will observe the set of channel intensities D^{F,r}ᵢ=(X^{F,r}ᵢ, Y^{F,r}ᵢ).

### Hypothesis Nomenclature

For SNP i, and target j, the hypothesis consists of the mother and father origin hypothesis, i.e. H^{Tj}ᵢ=(H^{Tj}_{i,m}, H^{Tj}_{i,f}), where H^{Tj}_{i,m} in {1, 2}, H^{Tj}_{i,f} in {1, 2}, each of which denote the parent haplotype of origin for each value. For sperm, there is only a father origin hypothesis, i.e. H^{Sj}ᵢ in {1, 2}, indicating paternal origin (assuming normal sperm).

Overall, one may write:

H=(H₁, . . . , Hₙ), where Hᵢ=(H^{T}ᵢ, H^{S}ᵢ) and H^{T}ᵢ=(H^{T1}ᵢ, H^{T2}ᵢ, . . . , H^{Tk}ᵢ) and H^{S}ᵢ=(H^{S1}ᵢ, H^{S2}ᵢ, . . . , H^{S1}ᵢ), where H^{Tj}ᵢ=(H^{Tj}_{i,m}, H^{Tj}_{i,f}).

In an example with 3 embryos and 1 sperm, a particular SNP hypothesis for one chromosomal segment could be ((M₁,P₂),(M₂,P₂),(M₂,P₁),S₁). There are total of 2^{(2k+1)n} different hypotheses H.

### Estimating Target Genotype Likelihood P(g|D)

For SNP i, target j, if P(g|D) is found, then the most likely ĝᵢ^{j}=argmax_{y}P(g|D), is picked as the allele call, with confidence oᵢ^{j}=P(ĝᵢ^{j}|D). In order to derive P(g|D), first let g^{M},g^{F} be possible ordered parents at the i^{th} SNP, i.e. g^{M},g^{F}ε{AA,AB,BA,BB}. Hᵢ is the full hypothesis on SNP i. Thus:

$$P ( {g}_{i}^{j} \vert D ) \sim {\sum }_{{H}_{i}} P ( {g}_{i}^{j} , {H}_{i} , D ) = {\sum }_{{H}_{i}} P ( D \text{?} \vert {H}_{i} ) P ( D \text{?} \vert {N}_{i} ) P ( {D}_{i} , {g}_{i}^{j} , {H}_{i} )$$

$$\text{?} \text{indicates text missing or illegible when filed}$$

Here the probability has been divided into the local probabilities of data on SNP i, (Dᵢ,gᵢ^{j},Hᵢ).

and the probabilities for data on all other SNPs only depends on the hypothesis Hᵢ:

P(D_{1, . . . ,i-1}|Hᵢ),P(D_{i+1, . . . ,n}|Hᵢ).

### The Probability on SNP i

$$P ( {D}_{i} , {g}_{i}^{j} , {H}_{i} ) = {\sum }_{\text{?}} P ( {D}_{ij} , {g}_{i}^{j} , {H}_{i} , {g}^{M} , {g}^{F} ) == {\sum }_{\text{?}} P ( {D}_{i} \vert {g}_{i}^{j} , {g}^{M} , {g}^{F} , {H}_{i} ) P ( {g}_{i}^{j} \vert {g}^{M} , {g}^{F} , {H}_{i}^{{T}_{j}} ) P ( {g}^{F} ) P ( {H}_{i} )$$

$$\text{?} \text{indicates text missing or illegible when filed}$$

P(g^{M}),p(G^{F}) are allele frequencies of ordered parent alleles on this SNP. In particular if on this SNP P(A)=p, then P(AA)=p², P(AB)=P(BA)=p(1−p), P(BB)=(1−p)². SNP allele frequencies may be estimated separately from large samples of genomic data.

P(Hᵢ) is generally same for all hypotheses Hᵢ, and on all SNPs, except that for one of the SNPs (this may be chosen arbitrarily; one may choose a SNP in the middle, say on SNP n/2), the hypothesis is restricted and the first target may be called (M₁,F₁) for uniqueness.

P(gᵢ^{j}|g^{M},g^{F},Hᵢ^{Tj}) is 1 or 0, depending on agreement of allele value gᵢ^{j} and one produced by a combination of g^{M},g^{F}, Hᵢ^{Tj}, i.e. if we define α(g^{M},g^{F},h)=(an allele value uniquely defined by ordered mother allele g^{M}, an ordered father allele g_{F}, and parent hypothesis h), then:

P(gᵢ^{j}|g^{M},g^{F},Hᵢ^{Tj})=I{gᵢ^{j}=α(g^{M},g^{F},Hᵢ^{Tj})}

Now P(Dᵢ|gᵢ^{j},g^{M},g^{F},Hᵢ) is the likelihood of data given particular allele values, since given parents g^{M},g^{F} and hypothesis Hᵢ, allele values for all targets, sperms and parents are uniquely determined. In particular it can be rewritten as:

P(Dᵢ|gᵢ^{j},g^{M},g^{F},Hᵢ)=P(Dᵢ^{T}|gᵢ^{j},g_{M},g^{F},Hᵢ^{T})P(Dᵢ^{F}|g^{F},Hᵢ^{F})P(Dᵢ^{M}|g^{M})P(Dᵢ^{F}|g^{F})

For targets:

P(Dᵢ^{T}|gᵢ^{j},g^{M},g^{F},Hᵢ^{T})=P(Dᵢ^{Tj}|gᵢ^{j})Π_{uεj}P(Dᵢ^{Tu}|α(g^{M},g^{F},Hᵢ^{Tu}))

For each target u, P(Dᵢ^{Tu}|g) is the product of likelihoods of all the resamples of that target

P(Dᵢ^{Tu}|g)=Πᵢ,P(Dᵢ^{Tur}|g).

Similarly for sperm:

P(Dᵢ^{s}|g^{F},Hᵢ^{S})=ΠᵤP(Dᵢ^{Su}|α(g^{F},Hᵢ^{Su}))

For each sperm u, P(Dᵢ^{Su}|g)) is the product of likelihoods of all the resamples of that sperm

P(Dᵢ^{Su}|g)=Πᵢ,P(Dᵢ^{Su,r}|g).

For parents, one may multiply likelihoods of resamples for each parent:

P(Dᵢ^{M}|g^{M})=Πᵢ,P(Dᵢ^{M,r}|g^{M}),P(Dᵢ^{F}|g^{F})=Πᵢ,P(Dᵢ^{F,r}|g^{F})

The piece of the likelihood P(D|g) remaining to be discussed, for each target, sperm and parent sample, is the estimated platform response model for that sample. This will be discussed later.

Probability on SNPs 1, . . . , i−1

For Hᵢ₋₁ all possible hypotheses on SNP i−1

$$P ( D \text{?} \vert {H}_{i} ) = {\sum }_{\text{?}} P ( D \text{?} \vert {E}_{i - 1} ) P ( {H}_{i - 1} \vert {H}_{i} ) = {\sum }_{\text{?}} P ( D \text{?} \vert {H}_{i - 1} ) P ( {D}_{i - 1} \vert {H}_{i - 1} ) P ( {H}_{i - 1} \vert {H}_{i} )$$

$$\text{?} \text{indicates text missing or illegible when filed}$$

P(D_{1, . . . ,i-1}|Hᵢ₋₁), is of the same format as P(D_{1, . . . ,i-1}|Hᵢ), and can be calculated sequentially going up from SNP 1. In particular, define matrix Wⁱ as Wⁱ(h,1)=P(D_{1, . . . ,i-1}|h) where h is the hypothesis on SNP i. Define matrix PDⁱ as PDⁱ⁻¹(g,1)=P(Dᵢ₋₁|g) where g is the hypothesis on SNP i−1. Define matrix PCⁱ as PCⁱ(h,g)=P(g|h), the probability of transition between hypotheses g to h, when going from SNP i−1 to i.

Then one may say Wⁱ=PCⁱ×(PDⁱ⁻¹·Wⁱ⁻¹) with the initial condition W¹(g)=P(start⊕g). This may be an arbitrary chosen constant.

So, first find W²=PC²×(PD¹·W¹), then W², and so on, go up to Wⁱ.

PCⁱ(Hᵢ,Hᵢ₋₁)=P(Hᵢ₋₁|Hᵢ) is the transition probability depending on the crossover probability between SNPs i−1, i. It is important to remember that hypothesis Hᵢ (and similarly for Hᵢ₋₁) consists of the hypothesis for all targets and sperm Hᵢ=(H^{T}ᵢ, H^{S}ᵢ). Hypothesis H^{T}ᵢ=(H^{T}₁, H^{T2}ᵢ, . . . , H^{Tk}ᵢ) are the target hypothesis for k targets, where each target hypothesis consists of the hypothesis of mother and father origin H^{Tj}ᵢ=(H^{Tj}_{i,m}, H^{Tj}_{i,f}). Hypothesis H^{S}ᵢ=(H^{S1}ᵢ, H^{S2}ᵢ, . . . , H^{Sl}ᵢ) is the father origin hypothesis for l sperms.

Then

P(Hᵢ₋₁|Hᵢ)=llⱼP(H_{i-1,m}^{Tj}|H_{i,m}^{Tj})llⱼP(H_{i-1,j}^{Tj}|H_{i,j}^{Tj})llⱼP(H_{i-1,j}^{Sj}|H_{i,j}^{Sj})

where P(g|h)={_{1-cp}^{cp}_{g=h}^{g≠h}, and where cp is the crossover probability between SNPs i,i−1, and may be estimated separately from HAPMAP data.

PDⁱ⁻¹(Hᵢ₋₁)=P(Dᵢ₋₁|Hᵢ₋₁) is the likelihood of data on SNP i−1, given this hypothesis Hᵢ₋₁, and it may be calculated by summing over all the ordered parent allele values, similar to breakdown described earlier.

$$P ( {D}_{i - 1} \vert {H}_{i - 1} ) = {\sum }_{\text{?}} P ( {D}_{i - 1} \vert {H}_{i - 1} , {g}^{M} , {g}^{F} ) P ( {g}^{M} ) P ( {g}^{F} ) = {\sum }_{\text{?}} P ( D \text{?} \vert {g}^{M} , {g}^{F} , {H}_{i - 1}^{T} ) P ( {D}_{i - 1}^{M} \vert {g}^{F} , {H}_{i - 1}^{x} ) P ( {D}_{i - 1}^{M} \vert {g}^{M} ) P ( {D}_{i - 1}^{F} \vert {g}^{F} ) P ( {g}^{M} ) P ( {g}^{F} )$$

$$\text{?} \text{indicates text missing or illegible when filed}$$

Probability on SNPs i+1, . . . , n

The derivation in this section is similar to the one above, except one goes from the other end, i.e. if we define Vⁱ(h,1)=P(D_{i+1, . . . ,n}|h), where h is the hypothesis on SNP I, then we have Vᵢ=PCⁱ⁺¹,Vⁱ⁺¹.

With initial condition Vⁿ(g)=P(end⊕g) (just constant same for all, unimportant) So, first find Vₙ₋₁=PCⁿX(PDⁿ,Vⁿ), and so on, go down to Vⁱ.

### Estimating Hypothesis P(h|D)

Deriving the exact target or sperm hypothesis is not integral to allele calling, but it may be very useful for result checking and other applications. The procedure is very similar to deriving genotype probabilities, and is outlined here. In particular, for SNP i, target j, and hypothesis h defined as particular hypothesis for SNP i, target j,

$$P ( \text{?} \vert D ) \sim {\sum }_{\text{?}} P ( D , {H}_{i} ) = {\sum }_{\text{?}} P ( D \text{?} \vert {H}_{i} ) P ( D \text{?} \vert {H}_{i} ) P ( {D}_{i} \vert {H}_{i} ) P ( {H}_{i} )$$

$$\text{?} \text{indicates text missing or illegible when filed}$$

where all the pieces are derived as described elsewhere in this document.

### Estimating Parent Genotype P(g D)

Deriving exact parent genotype is not integral to allele calling, but it may be very useful for result checking and other applications. The procedure is very similar to deriving genotype probabilities, and is outlines here. In particular, for SNP i, target j, say mother genotype gm

$$P ( {g}^{M} \vert D ) \sim {\sum }_{\text{?}} P ( D , {H}_{i} , {g}^{M} , {g}^{F} ) = {\sum }_{\text{?}} P ( D \text{?} \vert {H}_{i} ) P ( D \text{?} \vert {H}_{i} ) P ( {D}_{i} \vert {H}_{i} , {g}^{M} , {g}^{F} ) P ( {H}_{i} ) P ( {g}^{M} ) P ( {g}^{F} )$$

$$\text{?} \text{indicates text missing or illegible when filed}$$

where all the pieces are derived as described elsewhere in this document.

### Platform Response Model Estimating P(D^{T}|g)

The response model may be derived separately for each sample and each chromosome. The objective is to estimate of P((X,Y)|g) where g=AA, AB, BB.

First make discrete the range of X,Y intensity response into T bins B^{x},B^{Y}, derived as T equally spaced percentiles of data on respective channels (T<=20). Then one may estimate P((X,Y)|g) as P((X,Y)|g)˜f(bₓ,b_{y},g) for Xεbₓ,Yεb_{y}, where f(bₓ,b_{y},g) is estimated from data. In one embodiment the data may come from ILLUMINA SNP genotyping array output data and/or sequence data, which have different models. In other embodiments, the data may come from other genotyping arrays, from other sequencing methods, or other sources of genetic data.

### Model for ILLUMINA Data

From parent data, estimate the mother genotype G^{M}, the father genotype G^{F} and derive sample parent frequency p(gm,gf) for gm,gf=AA,AB,BB.

Estimate the allele frequency: P(g)˜f(g)=Σ_{gm,gf}P(g|gm,gf)·\hat{p}(gm,gf)

Define S^{AA} as the subset of SNPs of target data S for parental context AA|AA, i.e. S^{AA}={S|G^{M}=AA, G^{F}=AA}, and S^{BB} as the subset of SNPs of target data S for parental context BB|BB, i.e. S^{BB}={S|G^{M}=BB, G^{F}=BB. The allele value of SNPs in S^{AA} has to be AA, and similarly BB for S^{BB}.

### Joint Estimate

Define f^{joint}(bₓ,b_{y},AA) as the joint bin sample frequency of intensities in S^{AA}. This is an estimate of P((X,Y)|AA).

Define f^{joint}(bₓ,b_{y},BB) as the joint bin sample frequency of intensities in S^{BB}. This is an estimate of P((X,Y)|BB).

Define f^{joint}(bₓ,b_{y},:) as the joint bin sample frequency of intensities in S. This is an estimate of P((X,Y)).

Now, it is know that P((X,Y))=Σ_{g=AA,AB,BB}P((X,Y)|g)·P(g)

thus one may write

$$P ( ( X , Y ) \vert AB ) - \frac{P ( ( XX ) ) - P ( AA ) P ( ( X , Y ) \vert AA ) = P ( BB ) P ( ( XY ) \vert BB )}{1 - P ( AA ) - P ( BB )}$$

and it is possible to estimate P((X,Y)|AB) as follows:

$${f}^{joint} ( {b}_{x} , {b}_{y} , AB ) = \frac{{f}^{joint} ( {b}_{x} , {b}_{y} \text{?} ( AA ) {f}^{joint} ( {b}_{x} , {b}_{y} AA ) - f ( BB ) {f}^{joint} ( {b}_{x} , {b}_{y} \text{?} )}{1 - f ( AA ) - f ( BB )}$$

$$\text{?} \text{indicates text missing or illegible when filed}$$

Now the function f^{joint}(bₓ,b_{y},g) is one possible estimate of P((X,Y)|g).

### Marginal Estimate

Define f^{marginal}(bₓ,:,g) as the marginal bin frequency of channel X intensities in S^{g}, for g=AA,BB,:. This is an estimate of P(X|g).

Define f^{marginal}(:,b_{y},g) as the marginal bin frequency of channel Y intensities in S^{g}, for g=AA,BB,:. This is an estimate of P(Y|g).

If channel responses are assumed to be independent (which they may not be), the for g=AA,BB, one may write:

f^{marginal}(bₓ,b_{y},g)=f^{marginal}(bₓ,i,g)·f^{marginal}(i,b_{y}g)

and as before:

$${f}^{marginal} ( {b}_{x} , {b}_{y} , AB ) = \frac{\begin{matrix}{f}^{marginal} ( {b}_{x} {b}_{y} \text{?} - \\ f ( AA ) {f}^{marginal} ( {b}_{x} {b}_{y} AA ) - f ( Bb ) {f}^{marginal} ( {b}_{x} {b}_{y} BB )\end{matrix}}{i - f ( AA ) - f ( BB )}$$

$$\text{?} \text{indicates text missing or illegible when filed}$$

Now the function f^{marginal}(bₓ,b_{y},g) is another possible estimate of P((X,Y)|g).

### Combined Estimate

In some embodiments, for example, where f^{joint} is too data driven, and f^{marginal} is too smooth, i.e. not taking into account channel dependency, it is possible to use the combined estimate, pooling these two to give:

f(bₓ,b_{y},g)=c·f^{joint}(bₓ,b_{y},g)+(1−c)·f_{marginal}(bₓ,b_{y},g),

for c=0.5 (an arbitrary constant).

### Model for Sequence Data

Sequence data is different from data that originates from genotyping arrays. Each SNP is given separately, together with a plurality of locations around that SNP (typically about 400-500), by intensity for all 4 channels A,C,T,G. Sequence data also includes homozygous ‘wild’ call for all these locations. Typically, most of the non-SNP locations are homozygous and correspond to the wild call allele value. In one embodiment it is possible to assume that, for non-SNP locations, that wild call is the ‘truth’.

Call non-SNP intensity data ‘location data’ may be used to help build the response model. Location data is of the format LD=(LD₁, . . . , LDₙ) for n locations, where LDᵢ=(L^{A}ᵢ, L^{C}ᵢ, L^{T}ᵢ, L^{G}ᵢ), A,C,T,G intensities on location i. Corresponding wild call data is WD=(W₁, . . . , Wₙ), where Wᵢ is one of the A,C,T,G. Ideally, if a particular allele, say C, is present at location i, the intensity value, L^{C}ᵢ should be high. If the allele value is not present, its intensity should be very low, ideally 0. So, for example for TT, one may expect to have intensities for (A, T, C, G)=(low, high, low, low)=(no, yes, no, no). For AT, one may expect to have (high, high, low, low)=(yes, yes, no, no).

With this in mind, it is possible to estimate

f(bₓ,b_{y},AA)=YD(bₓ)·ND(bₓ), (yes on A, no on B)

f(bₓ,b_{y},AB)=YD(bₓ)·YD(bₓ), (yes on A, yes on B)

f(bₓ,b_{y},BB)=ND(bₓ)·YD(bₓ), (no on A, yes on B)

where YD(b) is the ‘yes’/present′ and ND(b) is the ‘no/absent’ one dimensional discrete bin distribution derived from data. YD may be derived from data in Yset={all channel intensities specified by wild call}. ND may be derived from data in Nset={all channel intensities NOT specified by wild call}. For example if the intensity at a particular location is (la, lc, lt, lg) and wild call is T, then lt will go toward Yset, and la, lc, lg will go toward Nset.

If channel independence and identical distribution (I.I.D. model) are assumed, then YD, ND distributions are just simple sample frequency of data in Yset, Nset respectively.

However, all four channels may be under- or over-amplified, and are therefore not independent. In one embodiment, it is possible to build a channel dependent and identical distribution (D.I.D. model), by scaling the intensity by maximum channel intensity on that location and applying I.I.D. model.

### Results

This section discusses the results of this allele calling method, as applied to real data, operating on a set of measured genetic data from related individuals. The input data consisted of the raw output from an ILLUMINA Infinium genotyping array. The data included 22 chromosomes, of 1000 SNP each, for one set of related individuals, including:
2 children (with 2 samples for each child),
3 embryos (2 samples for each embryo),
both parents (the mother and father, 2 genomic samples for each parent)
3 sperm (1 sample each)

### Target Calling Results

The overall hit rates given for children, where genomic measurements made on bulk tissue samples were considered to be the ‘truth’, was 98.55%. The hit rate varied for different contexts, and is given in the Table 4 below:

TABLE 4
Overall hit rates given for children

| (m₁m₂\|f₁f₂) | hit rate | standard deviation |
|---|---|---|
| AA\|AA | 0.9963 | σ = 0.1822 |
| AA\|AB | 0.9363 | σ = 0.0933 |
| AA\|BB | 0.9995 | σ = 0.0365 |
| AB\|AA | 0.9665 | σ = 0.0956 |
| AB\|AB | 0.9609 | σ = 0.1313 |
| AB\|AA | 0.9635 | σ = 0.1013 |
| BB\|AA | 0.9980 | σ = 0.0337 |
| BB\|AB | 0.9940 | σ = 0.1088 |
| BB\|BB | 0.9983 | σ = 0.2112 |

The hit rate varied by chromosome, and ranged from about 99.5% to about 96.4%. Chromosomes 16, 19 and 22 were below about 98%. Note that hit rates for the father derived SNPs was about 99.82%, and the hit rates for mother derived SNPs was about 93.75%. The better hit rates for the father derived SNPs is due to better father phasing thanks to the phased genetic data available by genotyping sperm.

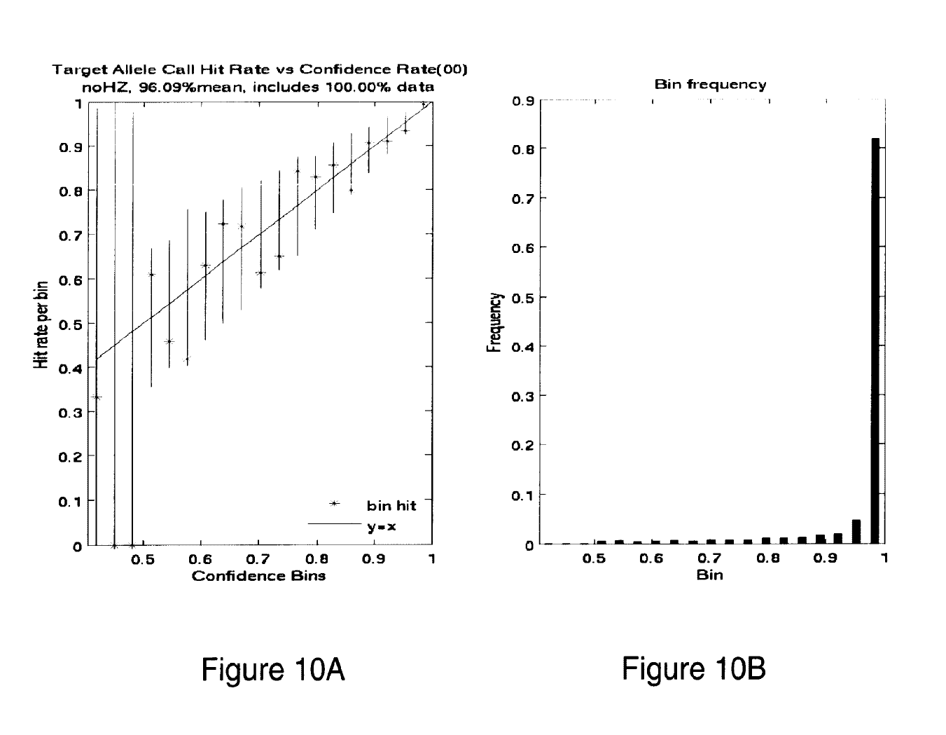

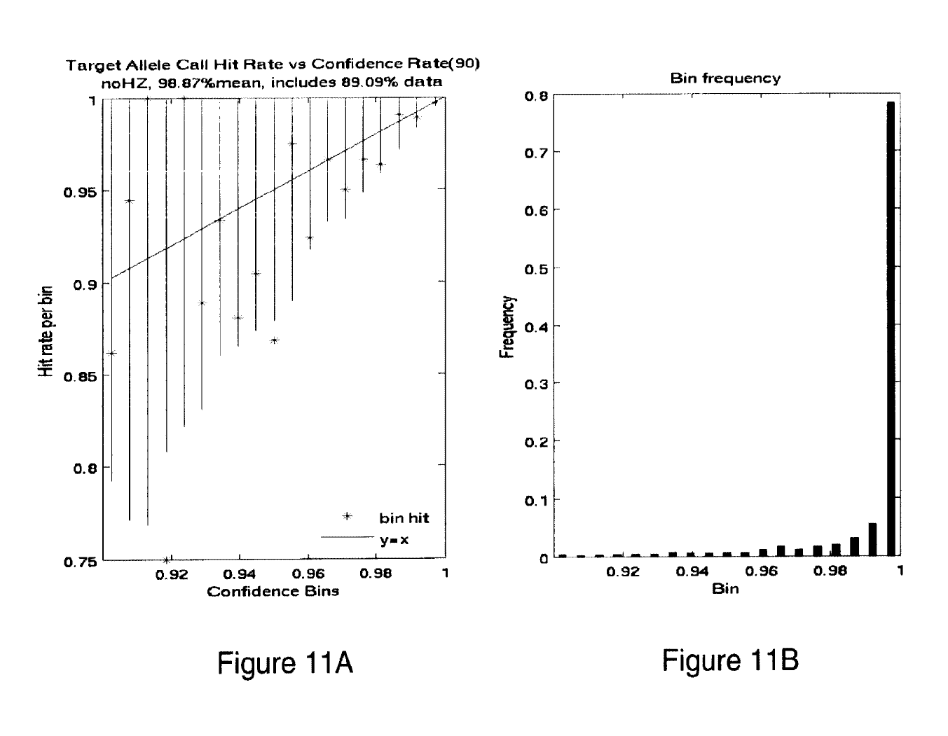

The hit rate by confidence bin refers to the hit rate for the set of allele calls that are predicted to have a certain confidence range. The overall hit rate for all of the data was about 98.55% hit rate. The hit rate for those allele calls which were predicted to have confidences above about 90%, which correspond to about 96.2% of all of the allele calls made, was 99.63%. The hit rate for those allele calls which were predicted to have confidences above about 99%, which corresponds to about 90.37% of the data, was about 99.9%. The hit rates for individual confidence bins indicate that the predicted confidences are quite accurate, within the limits of statistical significance. For example, for those allele calls with predicted confidences between about 80% and about 90% the actual hit rate was about 85.0%. For those allele calls with predicted confidences between about 70% and about 80% the actual hit rate was about 76.2%. For those allele calls with predicted confidences between about 96% and about 97% the actual hit rate was about 96.3%. For those allele calls with predicted confidences between about 94% and about 95% the actual hit rate was about 93.9%. For those allele calls with predicted confidences between about 99.1% and about 99.2% the actual hit rate was about 99.4%. For those allele calls with predicted confidences between about 99.8% and about 99.9% the actual hit rate was about 99.7%. FIGS. 10A and 10B and FIGS. 11A and 11B present plots of realized target hit rates, with confidence bars, versus hit rate as predicted by confidence. FIG. 10A plots the actual hit rate versus predicted confidence for bins that are three and a third percent wide, and FIG. 11A plots the actual hit rate versus predicted confidence for bins that are one half of a percent wide. The diagonal line represents the ideal case where the actual hit rate is equal to the predicted confidence. FIG. 10B shows the relative population of the various bin from FIG. 10A and FIG. 11B shows the relative population of the various bin from FIG. 11A. Bins with a higher population, or frequency, are expected to display a smaller deviation.

As a control, the same experiment was run, but genomic measurements taken on bulk data were used, instead of single cell measurements, as the measured target genetic data. In this case, the overall hit rate was about 99.88%.

Hypothesis Probability with Crossovers

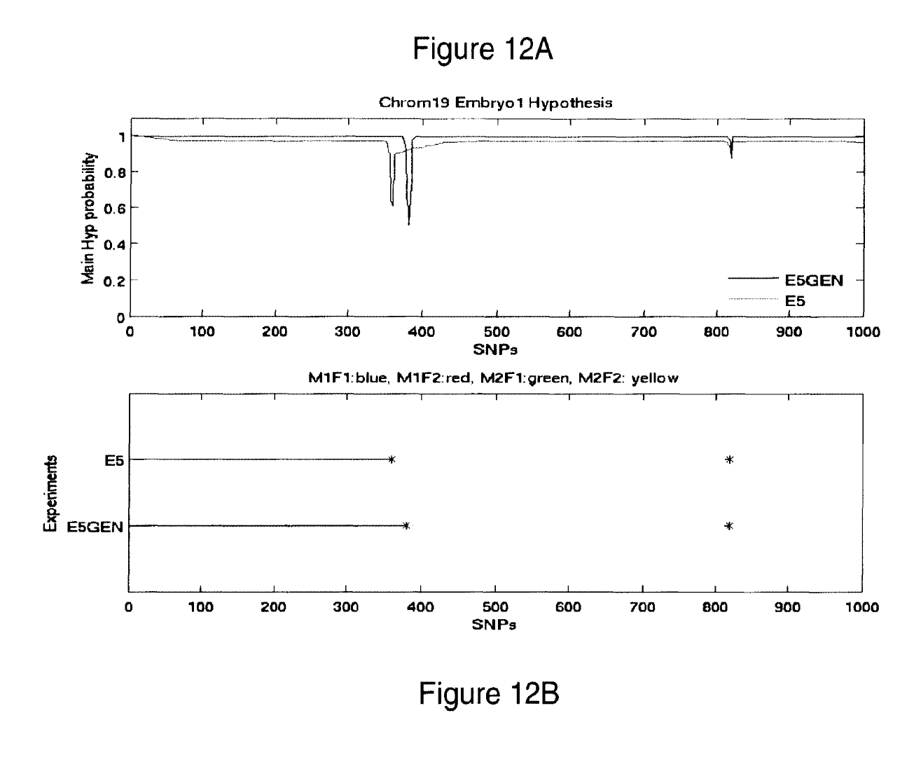

The method described herein is also able to determine whether a crossover occurred in the formation of the embryos. Since the accuracy of the allele calling relies on knowing the identity of neighboring alleles, one may expect that allele calls near a crossover, where the neighboring alleles may not be from the same haplotype, the confidence of those calls may drop. This can be seen in FIGS. 12A-12B. FIG. 12A shows the plot of allele confidence averaged over the neighboring SNPs for a typical chromosome. Two different sets of data are graphed, E5 and E5GEN, obtained from the same target individual, but using different methods. A sharp drop in confidence around a certain region of a chromosome is indicative of a crossover having occurred at the location during the meiosis that gave rise to the target individual. FIG. 12B shows a line depiction of the chromosome, with a star to indicate the location where the ploidy hypothesis has determined a crossover occurred. In FIG. 12B, it is possible to observe two crossovers, a crossover on the mother homolog around SNP 350, and crossover on the father homolog around SNP 820. The line denoted “E5” was when the method is run on single cell target data, and the line denoted “E5GEN” was when the method was run on genomic data measured on bulk tissue. The fact that the lines are similar indicates that the method is accurately reconstructing the genetic data of the single cell target, specifically, the crossover location.

### Varying the Number and Confidences of Input Data

In one embodiment of the present disclosure, it is possible to use genomic data from the mother and father, and single cell genetic data measured from the blastomeres and sperm. In another embodiment of the present disclosure, it is possible to also use genomic data from a born child from the same parents as additional information to help increase the accuracy of the determination of the single cell target genetic information. In one experiment, the genomic data of both parents along with the single cell genetic measurements from two embryo target cells were used, and the average hit rate on the target was about 95%. A similar experiment was run using the genomic data of both parents, the genomic data of one sibling, and the single cell target genetic information from one cell, and the added accuracy of the sibling genetic data increased the hit rate on the target cell to about 99%.

In another embodiment of the present disclosure, it is possible to use the genetic data from zero, one, two, three, four, or five or more sperm as input for the method. In some embodiments of the present disclosure is it possible to use the genetic data from one, two, three, four, five, or more than five sibling embryos as input for the method. In general, the bigger the number of inputs, the higher the accuracy of the target allele calls. Also, the higher the accuracy of the measurements of the inputs, the higher the accuracy of the target allele calls.

Another experiment was run with different sets of blastomere and sperm inputs, in the form of single cell blastomere measurements, and single cell sperm measurements. The Table 5 below shows that the higher the number of inputs, the higher the allele hit rate and hypothesis hit rate on the target. Note that “num sperms” indicates the number of sperm used in the determination; “num emb” corresponds to the total number of sibling embryos used in the determination, including the target; BK28 is a particular set of data.

TABLE 5
Number of inputs vs. allele hit rate and hypothesis hit rate on the target.

num sperms

| | | | | |
|---|---|---|---|---|
| num emb | 0 | 1 | 2 | 3 |

BK28 allele hit rate (%)

| | | | | |
|---|---|---|---|---|
| 3 | 93.46 | 95.18 | 95.69 | 95.86 |
| 4 | 95.06 | 96.13 | 96.59 | 96.75 |
| 5 | 95.93 | 96.67 | 97.00 | 97.15 |

BK28 hypothesis hit rate (%)

| | | | | |
|---|---|---|---|---|
| 3 | 98.49 | 99.72 | 99.73 | 99.74 |
| 4 | 99.70 | 99.72 | 99.73 | 99.73 |
| 5 | 99.64 | 99.65 | 99.52 | 99.68 |

### Amplification of Genomic DNA

Amplification of the genome can be accomplished by multiple methods including: ligation-mediated PCR (LM-PCR), degenerate oligonucleotide primer PCR (DOP-PCR), and multiple displacement amplification (MDA). Of the three methods, DOP-PCR reliably produces large quantities of DNA from small quantities of DNA, including single copies of chromosomes; this method may be most appropriate for genotyping the parental diploid data, where data fidelity is critical. MDA is the fastest method, producing hundred-fold amplification of DNA in a few hours; this method may be most appropriate for genotyping embryonic cells, or in other situations where time is of the essence.

Background amplification is a problem for each of these methods, since each method would potentially amplify contaminating DNA. Very tiny quantities of contamination can irreversibly poison the assay and give false data. Therefore, it is critical to use clean laboratory conditions, wherein pre- and post-amplification workflows are completely, physically separated. Clean, contamination free workflows for DNA amplification are now routine in industrial molecular biology, and simply require careful attention to detail.

### Genotyping Assay and Hybridization

The genotyping of the amplified DNA can be done by many methods including MOLECULAR INVERSION PROBES (MIPs) such as AFFYMETRIX's GENFLEX TAG Array, microarrays such as AFFYMETRIX's 500K array or the ILLUMINA BEAD arrays, or SNP genotyping assays such as APPLIED BIOSCIENCE's TAQMAN assay. These are all examples of genotyping techniques. The AFFYMETRIX 500K array, MIPs/GENFLEX, TAQMAN and ILLUMINA assay all require microgram quantities of DNA, so genotyping a single cell with either workflow requires some kind of amplification.

In the context of pre-implantation diagnosis during IVF, the inherent time limitations are significant, and methods that can be run in under a day may provide a clear advantage. The standard MIPs assay protocol is a relatively time-intensive process that typically takes about 2.5 to three days to complete. Both the 500K arrays and the ILLUMINA assays have a faster turnaround: approximately 1.5 to two days to generate highly reliable data in the standard protocol. Both of these methods are optimizable, and it is estimated that the turn-around time for the genotyping assay for the 500k array and/or the ILLUMINA assay could be reduced to less than 24 hours. Even faster is the TAQMAN assay which can be run in three hours. For all of these methods, the reduction in assay time may result in a reduction in data quality, however that is exactly what the disclosed present disclosure is designed to address.

Naturally, in situations where the timing is critical, such as genotyping a blastomere during IVF, the faster assays have a clear advantage over the slower assays, whereas in cases that do not have such time pressure, such as when genotyping the parental DNA before IVF has been initiated, other factors will predominate in choosing the appropriate method. Any techniques which are developed to the point of allowing sufficiently rapid high-throughput genotyping could be used to genotype genetic material for use with this method.

### Methods for Simultaneous Targeted Locus Amplification and Whole Genome Amplification.

During whole genome amplification of small quantities of genetic material, whether through ligation-mediated PCR (LM-PCR), multiple displacement amplification (MDA), or other methods, dropouts of loci occur randomly and unavoidably. It is often desirable to amplify the whole genome nonspecifically, but to ensure that a particular locus is amplified with greater certainty. It is possible to perform simultaneous locus targeting and whole genome amplification.

In one embodiment, it is possible to combine the targeted polymerase chain reaction (PCR) to amplify particular loci of interest with any generalized whole genome amplification method. This may include, but is not limited to, preamplification of particular loci before generalized amplification by MDA or LM-PCR, the addition of targeted PCR primers to universal primers in the generalized PCR step of LM-PCR, and the addition of targeted PCR primers to degenerate primers in MDA.

### Platform Response

There are many methods that may be used to measure genetic data. None of the methods currently known in the art are able to measure the genetic data with 100% accuracy, rather there are always errors, or statistical bias, in the data. It may be expected that the method of measurement will introduce certain statistically predictable biases into the measurement. It may be expected that certain sets of DNA, amplified by certain methods, and measured with certain techniques may result in measurements that are qualitatively and quantitatively different from other sets of DNA, that are amplified by other methods, and/or measured with different techniques. In some cases these errors may be due to the method of measurement. In some cases this error may be due to the state of the DNA. In some cases this bias may be due to the tendency of some types of DNA to respond differently to a given genetic measurement method. In some cases, the measurements may differ in ways that correlate with the number of cells used. In some cases, the measurements may differ based on the measurement technique, for example, which sequencing technique or array genotyping technique is used. In some cases different chromosomes may amplify to different extents. In some cases, certain alleles may be more or less likely to amplify. In some cases, the error, bias, or differential response may be due to a combination of factors. In many or all of these cases, the statistical predictability of these measurement differences, termed the ‘platform response’, may be used to correct for these factors, and can result in data that with an accuracy that is maximized, and where each measurement is associated with an appropriate confidence.

The platform response may be described as a mathematical characterization of the input/output characteristics of a genetic measurement platform, such as TAQMAN or Infinium. The input to the channel is the amplified genetic material with any annealed, fluorescently tagged genetic material. The channel output could be allele calls (qualitative) or raw numerical measurements (quantitative), depending on the context. For example, in the case in which the platform's raw numeric output is reduced to qualitative genotype calls, the platform response may consist of an error transition matrix that describes the conditional probability of seeing a particular output genotype call given a particular true genotype input. In one embodiment, in which the platform's output is left as raw numeric measurements, the platform response may be a conditional probability density function that describes the probability of the numerical outputs given a particular true genotype input.

In some embodiments of the present disclosure, the knowledge of the platform response may be used to statistically correct for the bias. In some embodiments of the present disclosure, the knowledge of the platform response may be used to increase the accuracy of the genetic data. This may be done by performing a statistical operation on the data that acts in the opposite manner as the biasing tendency of the measuring process. It may involve attaching the appropriate confidence to a given datum, such that when combined with other data, the hypothesis found to be most likely is indeed most likely to correspond to the actual genetic state of the individual in question.

### Other Notes

As noted previously, given the benefit of this disclosure, there are more embodiments that may implement one or more of the systems, methods, and features, disclosed herein.

In some embodiments of the present disclosure, a statistical method may be used to remove the bias in the data due to the tendency for maternal alleles to amplify in a disproportionate manner to the other alleles. In some embodiments of the present disclosure, a statistical method may be used to remove the bias in the data due to the tendency for paternal alleles to amplify in a disproportionate manner to the other alleles. In some embodiments of the present disclosure, a statistical method may be used to remove the bias in the data due to the tendency for certain probes to amplify certain SNPs in a manner that is disproportionate to other SNPs.

Imagine the two dimensional space where the x-coordinate is the x channel intensity and the y-coordinate is the y channel intensity. In this space, one may expect that the context means should fall on the line defined by the means for contexts BB|BB and AA|AA. In some cases, it may be observed that the average contexts means do not fall on this lone, but are biased in a statistical manner; this may be termed “off line bias”. In some embodiments of the present disclosure, a statistical method may be used to correct for the offline bias in the data.

In some cases splayed dots on the context means plot could be caused by translocation. If a translocation occurs, then one may expect to see abnormalities on the endpoints of the chromosome only. Therefore, if the chromosome is broken up into segments, and the context mean plots of each segment are plotted, then those segments that lie on the of a translocation may be expected to respond like a true trisomy or monosomy, while the remaining segments look disomic. In some embodiments of the present disclosure, a statistical method may be used to determine if translocation has occurred on a given chromosome by looking at the context means of different segments of the chromosome.

In some cases, it may be desirable to include a large number of related individuals into the calculation to determine the most likely genetic state of a target. In some cases, running the algorithm with all of the desired related individuals may not be feasible due to limits of computational power or time. The computing power needed to calculate the most likely allele values for the target increases exponentially with the number of sperm, blastomeres, and other input genotypes from related individuals. In one embodiment, these problems may be overcome by using a method termed “subsetting”, where the computations may be divided into smaller sets, run separately, and then combined. In one embodiment of the present disclosure, one may have the genetic data of the parents along with that of ten embryos and ten sperm. In this embodiment, one could run several smaller sub-algorithms with, for example three embryos and three sperm, and then pool the results. In one embodiment the number of sibling embryos used in the determination may be from one to three, from three to five, from five to ten, from ten to twenty, or more than twenty. In one embodiment the number of sperm whose genetic data is known may be from one to three, from three to five, from five to ten, from ten to twenty, or more than twenty. In one embodiment each chromosome may be divided into two to five, five to ten, ten to twenty, or more than twenty subsets.

In one embodiment of the present disclosure, any of the methods described herein may be modified to allow for multiple targets to come from same target individual. This may improve the accuracy of the model, as multiple genetic measurements may provide more data with which the target genotype may be determined. In prior methods, one set of target genetic data served as the primary data which was reported, and the other served as data to double-check the primary target genetic data. This embodiment of the present disclosure is an improvement over prior methods in that a plurality of sets of genetic data, each measured from genetic material taken from the target individual, are considered in parallel, and thus both sets of target genetic data serve to help determine which sections of parental genetic data, measured with high accuracy, composes the embryonic genome. In one embodiment of the present disclosure, the target individual is an embryo, and the different genotype measurements are made on a plurality of biopsied blastomeres. In another embodiment, one could also use multiple blastomeres from different embryos, from the same embryo, cells from born children, or some combination thereof

In some embodiments of the present disclosure, the methods described herein may be used to determine the genetic state of a developing fetus prenatally and in a non-invasive manner. The source of the genetic material to be used in determining the genetic state of the fetus may be fetal cells, such as nucleated fetal red blood cells, isolated from the maternal blood. The method may involve obtaining a blood sample from the pregnant mother. The method may involve isolating a fetal red blood cell using visual techniques, based on the idea that a certain combination of colors are uniquely associated with nucleated red blood cell, and a similar combination of colors is not associated with any other present cell in the maternal blood. The combination of colors associated with the nucleated red blood cells may include the red color of the hemoglobin around the nucleus, which color may be made more distinct by staining, and the color of the nuclear material which can be stained, for example, blue. By isolating the cells from maternal blood and spreading them over a slide, and then identifying those points at which one sees both red (from the Hemoglobin) and blue (from the nuclear material) one may be able to identify the location of nucleated red blood cells. One may then extract those nucleated red blood cells using a micromanipulator, use genotyping and/or sequencing techniques to measure aspects of the genotype of the genetic material in those cells. In one embodiment of the present disclosure, one may then use an informatics based technique such as the ones described in this disclosure to determine whether or not the cells are in fact fetal in origin. In one embodiment of the present disclosure, one may then use an informatics based technique such as the ones described in this disclosure to determine the ploidy state of one or a set of chromosomes in those cells. In one embodiment of the present disclosure, one may then use an informatics based technique such as the ones described in this disclosure to determine the genetic state of the cells. When applied to the genetic data of the cell, PARENTAL SUPPORT™ could indicate whether or not a nucleated red blood cell is fetal or maternal in origin by identifying whether the cell contains one chromosome from the mother and one from the father, or two chromosomes from the mother.

In one embodiment, one may stain the nucleated red blood cell with a die that only fluoresces in the presence of fetal hemoglobin and not maternal hemoglobin, and so remove the ambiguity between whether a nucleated red blood cell is derived from the mother or the fetus. Some embodiments of the present disclosure may involve staining or otherwise marking nuclear material. Some embodiments of the present disclosure may involve specifically marking fetal nuclear material using fetal cell specific antibodies. Some embodiments of the present disclosure may involve isolating, using a variety of possible methods, one or a number of cells, some or all of which are fetal in origin. Some embodiments of the present disclosure may involve amplifying the DNA in those cells, and using a high throughput genotyping microarray, such as the ILLUMINA INFINIUM array, to genotype the amplified DNA. Some embodiments of the present disclosure may involve using the measured or known parental DNA to infer the more accurate genetic data of the fetus. In some embodiments, a confidence may be associated with the determination of one or more alleles, or the ploidy state of the fetus. Some embodiments of the present disclosure may involve staining the nucleated red blood cell with a die that only fluoresces in the presence of fetal hemoglobin and not maternal hemoglobin, and so remove the ambiguity between whether a nucleated red blood cell is derived from the mother or the fetus.

There are many other ways to isolate fetal cells from maternal blood, or fetal DNA from maternal blood, or to enrich samples of fetal genetic material in the presence of maternal genetic material. Some of these methods are listed here, but this is not intended to be an exhaustive list. Some appropriate techniques are listed here for convenience: using fluorescently or otherwise tagged antibodies, size exclusion chromatography, magnetically or otherwise labeled affinity tags, epigenetic differences, such as differential methylation between the maternal and fetal cells at specific alleles, density gradient centrifugation succeeded by CD45/14 depletion and CD71-positive selection from CD45/14 negative-cells, single or double Percoll gradients with different osmolalities, or galactose specific lectin method.

One embodiment of the present disclosure could be as follows: a pregnant woman wants to know if her fetus is afflicted with Down Syndrome, and if it will suffer from Cystic Fibrosis. A doctor takes her blood, and stains the hemoglobin with one marker so that it appears clearly red, and stains nuclear material with another marker so that it appears clearly blue. Knowing that maternal red blood cells are typically anuclear, while a high proportion of fetal cells contain a nucleus, he is able to visually isolate a number of nucleated red blood cells by identifying those cells that show both a red and blue color. The doctor picks up these cells off the slide with a micromanipulator and sends them to a lab which amplifies and genotypes ten individual cells. By looking at the genetic measurements, the PARENTAL SUPPORT™ is able to determine that six of the ten cells are maternal blood cells, and four of the ten cells are fetal cells. If a child has already been born to a pregnant mother, PARENTAL SUPPORT™ can also be used to determine that the fetal cell is distinct from the cells of the born child by making reliable allele calls on the fetal cells and showing that they are dissimilar to those of the born child. The genetic data measured from the fetal cells is of very poor quality, containing many allele drop outs, due to the difficulty of genotyping single cells. The clinician is able to use the measured fetal DNA along with the reliable DNA measurements of the parents to infer the genome of the fetus with high accuracy using PARENTAL SUPPORT. The clinician is able to determine both the ploidy state of the fetus, and the presence or absence of a plurality of disease-linked genes of interest.

In some embodiments of the present disclosure, a plurality of parameters may be changed without changing the essence of the present disclosure. For example, the genetic data may be obtained using any high throughput genotyping platform, or it may be obtained from any genotyping method, or it may be simulated, inferred or otherwise known. A variety of computational languages could be used to encode the algorithms described in this disclosure, and a variety of computational platforms could be used to execute the calculations. For example, the calculations could be executed using personal computers, supercomputers, a massively parallel computing platform, or even non-silicon based computational platforms such as a sufficiently large number of people armed with abacuses.

Some of the math in this disclosure makes hypotheses concerning a limited number of states of aneuploidy. In some cases, for example, only zero, one or two chromosomes are expected to originate from each parent. In some embodiments of the present disclosure, the mathematical derivations can be expanded to take into account other forms of aneuploidy, such as quadrosomy, where three chromosomes originate from one parent, pentasomy, etc., without changing the fundamental concepts of the present disclosure.

In some embodiments of the present disclosure, a related individual may refer to any individual who is genetically related, and thus shares haplotype blocks with the target individual. Some examples of related individuals include: biological father, biological mother, son, daughter, brother, sister, half-brother, half-sister, grandfather, grandmother, uncle, aunt, nephew, niece, grandson, granddaughter, cousin, clone, the target individual himself/herself/itself, and other individuals with known genetic relationship to the target. The term ‘related individual’ also encompasses any embryo, fetus, sperm, egg, blastomere, blastocyst, or polar body derived from a related individual.

In some embodiments of the present disclosure, the target individual may refer to an adult, a juvenile, a fetus, an embryo, a blastocyst, a blastomere, a cell or set of cells from an individual, or from a cell line, or any set of genetic material. The target individual may be alive, dead, frozen, or in stasis.

In some embodiments of the present disclosure, where the target individual refers to a blastomere that is used to diagnose an embryo, there may be cases caused by mosaicism where the genome of the blastomere analyzed does not correspond exactly to the genomes of all other cells in the embryo.

In some embodiments of the present disclosure, it is possible to use the method disclosed herein in the context of cancer genotyping and/or karyotyping, where one or more cancer cells is considered the target individual, and the non-cancerous tissue of the individual afflicted with cancer is considered to be the related individual. The non-cancerous tissue of the individual afflicted with the target could provide the set of genotype calls of the related individual that would allow chromosome copy number determination of the cancerous cell or cells using the methods disclosed herein.

In some embodiments of the present disclosure, as all living or once living creatures contain genetic data, the methods are equally applicable to any live or dead human, animal, or plant that inherits or inherited chromosomes from other individuals.

It is also important to note that the embryonic genetic data that can be generated by measuring the amplified DNA from one blastomere can be used for multiple purposes. For example, it can be used for detecting aneuploidy, uniparental disomy, sexing the individual, as well as for making a plurality of phenotypic predictions based on phenotype-associated alleles. Currently, in IVF laboratories, due to the techniques used, it is often the case that one blastomere can only provide enough genetic material to test for one disorder, such as aneuploidy, or a particular monogenic disease. Since the method disclosed herein has the common first step of measuring a large set of SNPs from a blastomere, regardless of the type of prediction to be made, a physician, parent, or other agent is not forced to choose a limited number of disorders for which to screen. Instead, the option exists to screen for as many genes and/or phenotypes as the state of medical knowledge will allow. With the disclosed method, one advantage to identifying particular conditions to screen for prior to genotyping the blastomere is that if it is decided that certain loci are especially relevant, then a more appropriate set of SNPs which are more likely to co-segregate with the locus of interest, can be selected, thus increasing the confidence of the allele calls of interest.

In some embodiments, the systems, methods and techniques of the present disclosure may be used to decrease the chances that an implanted embryo, obtained by in vitro fertilization, undergoes spontaneous abortion.

In some embodiments of the present disclosure, the systems, methods, and techniques of the present disclosure may be used to in conjunction with other embryo screening or prenatal testing procedures. The systems, methods, and techniques of the present disclosure are employed in methods of increasing the probability that the embryos and fetuses obtain by in vitro fertilization are successfully implanted and carried through the full gestation period. Further, the systems, methods, and techniques of the present disclosure are employed in methods that may decrease the probability that the embryos and fetuses obtained by in vitro fertilization and that are implanted are not specifically at risk for a congenital disorder.

In some embodiments, the systems, methods, and techniques of the present disclosure are used in methods to decrease the probability for the implantation of an embryo specifically at risk for a congenital disorder by testing at least one cell removed from early embryos conceived by in vitro fertilization and transferring to the mother's uterus only those embryos determined not to have inherited the congenital disorder.

In some embodiments, the systems, methods, and techniques of the present disclosure are used in methods to decrease the probability for the implantation of an embryo specifically at risk for a chromosome abnormality by testing at least one cell removed from early embryos conceived by in vitro fertilization and transferring to the mother's uterus only those embryos determined not to have chromosome abnormalities.

In some embodiments, the systems, methods, and techniques of the present disclosure are used in methods to increase the probability of implantation of an embryo that was obtained by in vitro fertilization, is transferred, and that is at a reduced risk of carrying a congenital disorder.

In some embodiments, the congenital disorder is a malformation, neural tube defect, chromosome abnormality, Down's syndrome (or trisomy 21), Trisomy 18, spina bifida, cleft palate, Tay Sachs disease, sickle cell anemia, thalassemia, cystic fibrosis, Huntington's disease, Cri du chat syndrome, and/or fragile X syndrome. Chromosome abnormalities may include, but are not limited to, Down syndrome (extra chromosome 21), Turner Syndrome (45X0) and Klinefelter's syndrome (a male with 2 X chromosomes).

In some embodiments, the malformation may be a limb malformation. Limb malformations may include, but are not limited to, amelia, ectrodactyly, phocomelia, polymelia, polydactyly, syndactyly, polysyndactyly, oligodactyly, brachydactyly, achondroplasia, congenital aplasia or hypoplasia, amniotic band syndrome, and cleidocranial dysostosis.

In some embodiments, the malformation may be a congenital malformation of the heart. Congenital malformations of the heart may include, but are not limited to, patent ductus arteriosus, atrial septal defect, ventricular septal defect, and tetralogy of fallot.

In some embodiments, the malformation may be a congenital malformation of the nervous system. Congenital malformations of the nervous system include, but are not limited to, neural tube defects (e.g., spina bifida, meningocele, meningomyelocele, encephalocele and anencephaly), Arnold-Chiari malformation, the Dandy-Walker malformation, hydrocephalus, microencephaly, megencephaly, lissencephaly, polymicrogyria, holoprosencephaly, and agenesis of the corpus callosum.

In some embodiments, the malformation may be a congenital malformation of the gastrointestinal system. Congenital malformations of the gastrointestinal system include, but are not limited to, stenosis, atresia, and imperforate anus.

In some embodiments, the systems, methods, and techniques of the present disclosure are used in methods to increase the probability of implanting an embryo obtained by in vitro fertilization that is at a reduced risk of carrying a predisposition for a genetic disease.

In some embodiments, the genetic disease is either monogenic or multigenic. Genetic diseases include, but are not limited to, Bloom Syndrome, Canavan Disease, Cystic fibrosis, Familial Dysautonomia, Riley-Day syndrome, Fanconi Anemia (Group C), Gaucher Disease, Glycogen storage disease la, Maple syrup urine disease, Mucolipidosis IV, Niemann-Pick Disease, Tay-Sachs disease, Beta thalessemia, Sickle cell anemia, Alpha thalessemia, Beta thalessemia, Factor XI Deficiency, Friedreich's Ataxia, MCAD, Parkinson disease—juvenile, Connexin26, SMA, Rett syndrome, Phenylketonuria, Becker Muscular Dystrophy, Duchennes Muscular Dystrophy, Fragile X syndrome, Hemophilia A, Alzheimer dementia—early onset, Breast/Ovarian cancer, Colon cancer, Diabetes/MODY, Huntington disease, Myotonic Muscular Dystrophy, Parkinson Disease—early onset, Peutz-Jeghers syndrome, Polycystic Kidney Disease, Torsion Dystonia

### Combinations of the Aspects of the Present Disclosure

As noted previously, given the benefit of this disclosure, there are more aspects and embodiments that may implement one or more of the systems, methods, and features, disclosed herein. Below is a short list of examples illustrating situations in which the various aspects of the present disclosure can be combined in a plurality of ways. It is important to note that this list is not meant to be comprehensive; many other combinations of the aspects, methods, features and embodiments of this present disclosure are possible.

The key to one aspect of the present disclosure is the fact that ploidy determination techniques that make use of phased parental data of the target may be much more accurate than techniques that do not make use of such data. However, in the context of IVF, phasing the measured genotypic data obtained from bulk parental tissue is non-trivial. One method to determine the phased parental data from the unphased parental genetic data, along with the unphased genetic data from one or more embryos, zero or more siblings, and zero or more sperm is described in this disclosure. This method for phasing parental data assumes that the embryo genetic data is euploid at a given chromosome. Of course it may not be possible to determine the ploidy state at the given chromosome, to ensure euploidy, using a method that requires phased parental data as input, before that genetic data has been phased, presenting a boot strapping problem.

In some embodiments of the present disclosure, a method is disclosed herein wherein a technique for ploidy state determination is used to make a preliminary determination as to the ploidy state at a given chromosome for a set of cells derived from one or more embryos. Then, the method described herein for determining the phased parental data may be executed, using only the data from embryonic chromosomes that have been determined, with high confidence using the preliminary method, to be euploid. Once the parental data has been phased, then the ploidy state determination method that requires phased parental data may be used to give high accuracy ploidy determinations. The output from this method may be used on its own, or it may be combined with other ploidy determination methods.

Some of the expert techniques for copy number calling described in this disclosure, for example the “presence of homologues” technique, rely on phased parental genomic data. Some methods to phase data, such as some of those described in this disclosure, operate on the assumption that the input data is from euploid genetic material. When the target is a fetus or an embryo, it is particularly likely that one or more chromosomes are not euploid. In one embodiment of the present disclosure, one or a set of ploidy determination techniques that do not rely on phased parental data may be used to determine which chromosomes are euploid, such that genetic data from those euploid chromosomes may be used as part of an allele calling algorithm that outputs phased parental data, which may then be used in the copy number calling technique that requires phased parental data.

In one embodiment of the present disclosure, a method to determine the ploidy state of at least one chromosome in a target individual includes obtaining genetic data from the target individual, and from both parent of the target individual, and from one or more siblings of the target individual, wherein the genetic data includes data relating to at least one chromosome; determining a ploidy state of the at least one chromosome in the target individual and in the one or more siblings of the target individual by using one or more expert techniques, wherein none of the expert techniques requires phased genetic data as input; determining phased genetic data of the target individual, and of the parents of the target individual, and of the one or more siblings of the target individual, using an informatics based method, and the obtained genetic data from the target individual, and from the parents of the target individual, and from the one or more siblings of the target individual that were determined to be euploid at that chromosome; and redetermining the ploidy state of the at least one chromosome of the target individual, using one or more expert techniques, at least one of which requires phased genetic data as input, and the determined phased genetic data of the target individual, and of the parents of the target individual, and of the one or more siblings of the target individual. In an embodiment, the ploidy state determination can be performed in the context of in vitro fertilization, and where the target individual is an embryo. The determined ploidy state of the chromosome on the target individual can be used to make a clinical decision about the target individual.

First, genetic data may be obtained from the target individual and from the parents of the target individual, and possibly from one or more individuals that are siblings of the target individual. This genetic data from individuals may be obtained in a number of ways, and these are described elsewhere in this disclosure. The target individual's genetic data can be measured using tools and or techniques taken from a group including, but not limited to, MOLECULAR INVERSION PROBES (MIP), Genotyping Microarrays, the TAQMAN SNP Genotyping Assay, the ILLUMINA Genotyping System, other genotyping assays, fluorescent in-situ hybridization (FISH), sequencing, other high through-put genotyping platforms, and combinations thereof. The target individual's genetic data can be measured by analyzing substances taken from a group including, but not limited to, one or more diploid cells from the target individual, one or more haploid cells from the target individual, one or more blastomeres from the target individual, extra-cellular genetic material found on the target individual, extra-cellular genetic material from the target individual found in maternal blood, cells from the target individual found in maternal blood, genetic material known to have originated from the target individual, and combinations thereof. The related individual's genetic data can be measured by analyzing substances taken from a group including, but not limited to, the related individual's bulk diploid tissue, one or more diploid cells from the related individual, one or more haploid cells taken from the related individual, one or more embryos created from (a) gamete(s) from the related individual, one or more blastomeres taken from such an embryo, extra-cellular genetic material found on the related individual, genetic material known to have originated from the related individual, and combinations thereof

Second, a set of at least one ploidy state hypothesis may be created for one or more chromosome of the target individual and of the siblings. Each of the ploidy state hypotheses may refer to one possible ploidy state of the chromosome of the individuals.

Third, using one or more of the expert techniques, such as those discussed in this disclosure, a statistical probability may be determined for each ploidy state hypothesis in the set. In this step, the expert techniques is an expert technique that does not required phased genetic data as input. Some examples of expert techniques that do not require phased genetic data as input include, but are not limited to, the permutation technique, the whole chromosome mean technique, and the presence of parents technique. The mathematics underlying the various appropriate expert techniques is described elsewhere in this disclosure.

Fourth, if more than one expert method was used in the third step, then the set of determined probabilities may then be combined and normalized. The set of the products of the probabilities for each hypothesis in the set of hypotheses is then output as the combined probabilities of the hypotheses.

Fifth, the most likely ploidy state for the target individual, and for each of the sibling individual(s), is determined to be the ploidy state that is associated with the hypothesis whose probability is the greatest.

Sixth, an informatics based method, such as the allele calling method disclosed in this document, or other aspects of the PARENTAL SUPPORT™ method, along with unordered parental genetic data, and the genetic data of siblings that were found to be euploid in the fifth step, at that chromosome, may be used to determine the most likely allelic state of the target individual, and of the sibling individuals. In some embodiments, the target individuals may be treated the same, algorithmically, as the siblings. In some embodiments, the allelic state of a sibling may be determined by letting the target individual act as a sibling, and the sibling act as a target. In some embodiments, the informatics based method should also output the allelic state of the parents, including the haplotypic genetic data. In some embodiments of the present disclosure the informatics based method used may also determine the most likely phased genetic state of the parent(s) and of the other siblings.

Seventh, a new set of at least one ploidy state hypothesis may be created for one or more chromosome of the target individual and of the siblings. As before, each of the ploidy state hypotheses may refer to one possible ploidy state of the chromosome of the individuals.

Eighth, using one or more of the expert techniques, such as those discussed in this disclosure, a statistical probability may be determined for each ploidy state hypothesis in the set. In this step, at least one of the expert techniques is an expert technique that does require phased genetic data as input, such as the ‘presence of homologs’ technique.

Ninth, the set of determined probabilities may then be combined as described in the fourth step.

Lastly, the most likely ploidy state for the target individual, at that chromosome, is determined to be the ploidy state that is associated with the hypothesis whose probability is the greatest. In some embodiments, the ploidy state will only be called if the hypothesis whose probability is the greatest exceeds a certain threshold of confidence and/or probability.

In one embodiment of this method, in the third step, the following three expert techniques can be used in the initial ploidy state determination: the permutation technique, the whole chromosome mean technique, and the presence of parents technique. In one embodiment of the present disclosure, in the eighth step, the following set of expert techniques can be used in the final ploidy determination: the permutation technique, the whole chromosome mean technique, the presence of parents technique, and the presence of homologues technique. In some embodiments of the present disclosure different sets of expert techniques may be used in the third step. In some embodiments of the present disclosure different sets of expert techniques may be used in the eighth step. In one embodiment of the present disclosure, it is possible to combine several of the aspects of the present disclosure such that one could perform both allele calling as well as aneuploidy calling using one algorithm.

In an embodiment of the present disclosure, the disclosed method is employed to determine the genetic state of one or more embryos for the purpose of embryo selection in the context of IVF. This may include the harvesting of eggs from the prospective mother and fertilizing those eggs with sperm from the prospective father to create one or more embryos. It may involve performing embryo biopsy to isolate a blastomere from each of the embryos. It may involve amplifying and genotyping the genetic data from each of the blastomeres. It may include obtaining, amplifying and genotyping a sample of diploid genetic material from each of the parents, as well as one or more individual sperm from the father. It may involve incorporating the measured diploid and haploid data of both the mother and the father, along with the measured genetic data of the embryo of interest into a dataset. It may involve using one or more of the statistical methods disclosed in this patent to determine the most likely state of the genetic material in the embryo given the measured or determined genetic data. It may involve the determination of the ploidy state of the embryo of interest. It may involve the determination of the presence of a plurality of known disease-linked alleles in the genome of the embryo. It may involve making phenotypic predictions about the embryo. It may involve generating a report that is sent to the physician of the couple so that they may make an informed decision about which embryo(s) to transfer to the prospective mother.

Another example could be a situation where a 44-year old woman undergoing IVF is having trouble conceiving. The couple arranges to have her eggs harvested and fertilized with sperm from the man, producing nine viable embryos. A blastomere is harvested from each embryo, and the genetic data from the blastomeres are measured using an ILLUMINA INFINIUM BEAD array. Meanwhile, the diploid data are measured from tissue taken from both parents also using the ILLUMINA INFINIUM BEAD array. Haploid data from the father's sperm is measured using the same method. The method disclosed herein is applied to the genetic data of the nine blastomeres, of the diploid maternal and paternal genetic data, and of three sperm from the father. The methods described herein are used to clean and phase all of the genetic data used as input, plus to make ploidy calls for all of the chromosomes on all of the embryos, with high confidences. Six of the nine embryos are found to be aneuploid, and three embryos are found to be euploid. A report is generated that discloses these diagnoses, and is sent to the doctor. The doctor, along with the prospective parents, decides to transfer two of the three euploid embryos, one of which implants in the mother's uterus.

Another example may involve a pregnant woman who has been artificially inseminated by a sperm donor, and is pregnant. She is wants to minimize the risk that the fetus she is carrying has a genetic disease. She has blood drawn at a phlebotomist, and techniques described in this disclosure are used to isolate three nucleated fetal red blood cells, and a tissue sample is also collected from the mother and father. The genetic material from the fetus and from the mother and father are amplified as appropriate, and genotyped using the ILLUMINA INFINIUM BEAD array, and the methods described herein clean and phase the parental and fetal genotype with high accuracy, as well as to make ploidy calls for the fetus. The fetus is found to be euploid, and phenotypic susceptibilities are predicted from the reconstructed fetal genotype, and a report is generated and sent to the mother's physician so that they can decide what actions may be best.

Another example could be a situation where a racehorse breeder wants to increase the likelihood that the foals sired by his champion racehorse become champions themselves. He arranges for the desired mare to be impregnated by IVF, and uses genetic data from the stallion and the mare to clean the genetic data measured from the viable embryos. The cleaned embryonic genetic data allows the breeder to select the embryos for implantation that are most likely to produce a desirable racehorse.

A method for determining a ploidy state of at least one chromosome in a target individual includes obtaining genetic data from the target individual and from one or more related individuals; creating a set of at least one ploidy state hypothesis for each of the chromosomes of the target individual; determining a statistical probability for each ploidy state hypothesis in the set given the obtained genetic data and using one or more expert techniques; combining, for each ploidy state hypothesis, the statistical probabilities as determined by the one or more expert techniques; and determining the ploidy state for each of the chromosomes in the target individual based on the combined statistical probabilities of each of the ploidy state hypotheses.

A method for determining allelic data of one or more target individuals, and one or both of the target individuals' parents, at a set of alleles, includes obtaining genetic data from the one or more target individuals and from one or both of the parents; creating a set of at least one allelic hypothesis for each of the alleles of the target individuals and for each of the alleles of the parents; determining a statistical probability for each allelic hypothesis in the set given the obtained genetic data; and determining the allelic state for each of the alleles in the one or more target individuals and the one or both parents based on the statistical probabilities of each of the allelic hypothesis.

A method for determining a ploidy state of at least one chromosome in a target individual includes obtaining genetic data from the target individual, from both of the target individual's parents, and from one or more siblings of the target individual, wherein the genetic data includes data relating to at least one chromosome; determining a ploidy state of the at least one chromosome in the target individual and in the one or more siblings of the target individual by using one or more expert techniques, wherein none of the expert techniques requires phased genetic data as input; determining phased genetic data of the target individual, of the parents of the target individual, and of the one or more siblings of the target individual, using an informatics based method, and the obtained genetic data from the target individual, from the parents of the target individual, and from the one or more siblings of the target individual that were determined to be euploid at that chromosome; and redetermining the ploidy state of the at least one chromosome of the target individual, using one or more expert techniques, at least one of which requires phased genetic data as input, and the determined phased genetic data of the target individual, of the parents of the target individual, and of the one or more siblings of the target individual.

### Further Discussion of Embodiments of the Present Invention

In an embodiment, the present disclosure may be used to enable a clinician, or other agent, to identify one or more embryos, from among a set of embryos, that are the most likely to develop as desired. Typically, embryos that test negative for chromosomal abnormalities, such as aneuploidy, may be chosen for transfer. However, in some cases, there may be insufficient or no embryos that test negative for chromosomal abnormalities such as aneuploidy. In this case, embryos from which one cell has tested positive for a chromosomal abnormality may be aneuploid, or they may be mosaic. Mosaic cells may self correct, and have the potential to implant and develop as desired. In an embodiment, the present disclosure may be used to determine which embryo(s) are most likely to develop as desired. In an embodiment, the grouping or relative ranking of embryos may be made based on a model of mosaicism and how it arises during the development of the embryo.

Within an embryo, different distributions of cells of different ploidy states may occur, and embryos with some of those distributions are more likely than others to develop as desired. An embodiment may utilize the measured genetic condition in one cell from one or more embryo to predict the likely genetic condition in the remaining cells in the embryo. In this embodiment, the genetic condition may be the ploidy state. This measurement may be used to determine whether the cells of an embryo are likely to be euploid, aneuploid, or mosaic, and hence the relative likelihood of that embryo to develop as desired.

In an embodiment of the present disclosure, the present method may assume that the rates of aneuploidy and mosaicism may tend to increase as an embryo develops from the 2 cell to the 8 cell stage. This embodiment may also assume that aneuploidy in embryos often may be accompanied by mosaicism. In an embodiment, the above assumptions may be used to determine the distribution of aneuploidy states in one or more cells from an embryo. In an embodiment, the method may also assume that mosaicism is caused predominantly by errors in mitotic disjunction during embryo growth.

For example, consider that each chromosome has a probability of a non-disjunction error during mitosis. Each time a disjunction error occurs during the mitosis of a cell that is euploid at a given chromosome, that chromosome will have 0 copies of that chromosome in one of the post-division cells and 2 identical copies of that chromosome in the other post-division cell; therefore, both of these post-division cells are now aneuploid. If no error occurs, a chromosome will have 1 copy of each of the identical chromosomes in each of the two post-division cells. Further divisions of such an aneuploid cell will result in daughter aneuploid cells, with the exception of the unlikely event that a non-disjunction error occurs during the division of a cell that is trisomic at a chromosome that results in one of the duplicated identical chromosomes not being passed on to the daughter cell.

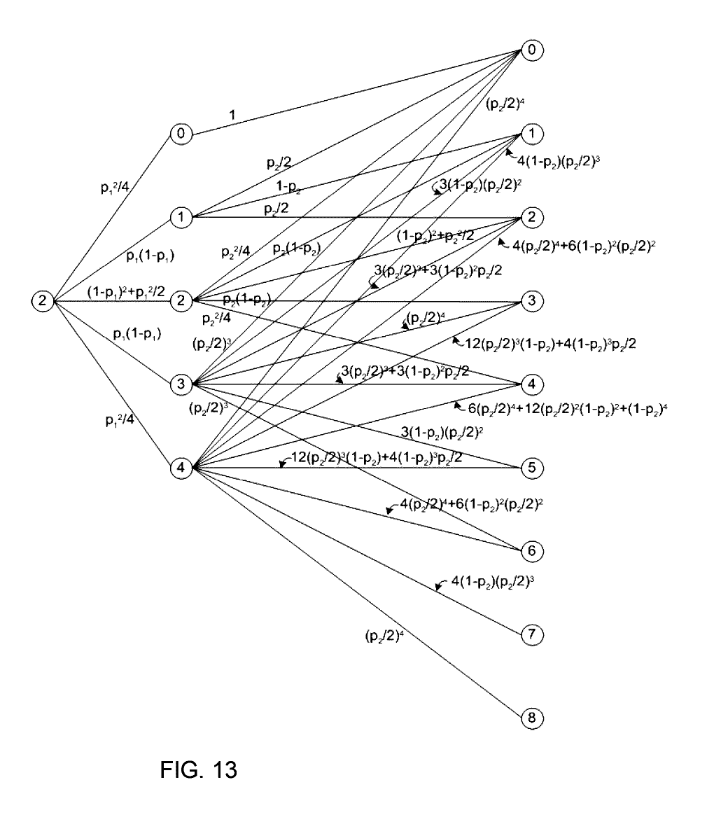

FIG. 13 is a graphical illustration of how, after two divisions, there will be a distribution of probabilities on each of the possible copy numbers of a particular chromosome in a cell. The number of copies of the chromosome is shown in the circles, and the lines between circles represent the transition probability of going from some number of chromosomes to the other during a division. The circle on the left represents a euploid parent cell. The column of circles in the middle represent the possible ploidy states of that chromosome after one division, and the column of circles on the right represent the possible ploidy states after two divisions. One may assume that the probability of a non-disjunction error is the same for each chromosome and that the probability is independent of the number of chromosomes in the pre-division cell. For the first division, the probability of a non-disjunction error is p₁ and for the second division the probability is p₂.

The ploidy state of a cell may be measured using the assumption that most errors occur during the first two cell divisions for a series of cells on day 3 embryos. The resulting measurements can be matched with the results of the model in order to estimate p₁ and p₂. Using the transition probabilities illustrated in FIG. 13, it may be possible to compute the probability of each of the possible ploidy states for that chromosome (1 through 8) in terms of p₁ and p₂. Each of these possible states may be considered hypotheses. In one embodiment, these computed probabilities may be compared with the empirical probabilities on each of the measured chromosome numbers in order to solve for p₁ and p₂ that most closely fit the data under a maximum-likelihood algorithm.

One relevant parameter from this analysis is r₁₂=p₁/p₂, describing the ratio of the probabilities of a mitotic disjunction error in the first and second division. If r₁₂ is close to 1, the distinction between p₁ and p₂ may be eliminated and the disjunction error at each division can be characterized simply as p. This model may be extended to incorporate errors at the third division (the probability of which is indicated by p₃). The model in FIG. 13 may be extended to a third or later division by algebraic methods, or by automated computer simulation, for example using a Monte Carlo method. In one embodiment of the present disclosure, this method may be used to calculate the likelihood of various ploidy states by modeling potential disjunction errors over fewer than two divisions. In an embodiment, this method may be used to calculate the likelihood of various ploidy states by modeling potential disjunction errors over two divisions. In one embodiment, the method can be used to calculate the likelihood of various ploidy states by modeling disjunction errors over three divisions. In another embodiment, the method can be used to calculate the likelihood of various ploidy states by modeling disjunction errors over four, five, six, seven or more divisions.

For the purpose of explanation, one may assume that the first division represents the first mitotic division after the completion of Meiosis II and the extrusion of the polar body following fertilization of an egg by a sperm. Disjunction errors that affect the formation of the sperm or the egg will tend to give rise to cells with additional chromosomes that do not exactly match other chromosomes because crossovers were involved in their formation which are different to the crossovers that gave rise to the other chromosomes in the post-division cell. However, disjunction errors in the divisions illustrated in FIG. 13 will give rise to cells with chromosomes that are exact copies of other chromosomes in the post-division cell. These are referred to as matching chromosomes aneuploidies, or MCAs. If the error occurs before the divisions in FIG. 13, either affecting the sperm or the egg or the fertilized egg, then it is likely that this would cause a unique chromosome aneuploidy, or a UCA.

In one embodiment of the present disclosure, a mechanism that may be used to explain mosaicism in embryos is used, together with the determination of one or more characteristic made on one or more cells, in order to determine one or more characteristic of other, untested cells within the embryo. If the egg or sperm is affected by an aneuploidy, then it is likely that all blastomeres in the embryo will be affected. Hence, if a UCA is measured, then the embryo has a relatively low probability of having any normal cells; if an MCA is measured, then there is a relatively high probability that the embryo contains some normal cells. In one embodiment of the present disclosure, the one or more characteristic may include the genetic condition of the one or more cells. In one embodiment, the one or more characteristic may include the ploidy state of one or more cells. In one embodiment of the present disclosure, a method, such as PARENTAL SUPPORT™, may be used to determine the subcharacteristics of the one or more cells, such as the type of aneuploidy in a cell.

An embodiment of the present disclosure may include a method of characterizing an embryo for insertion into a uterus, comprising: selecting at least one characteristic; determining a first at least one characteristic of at least one cell from an embryo; using the determined first characteristic, predicting a probability of a second cell from the embryo having a second characteristic; and characterizing the embryo based on the predicted probability. In an embodiment of the present disclosure, the determination step is performed on more than one cell from an embryo. In an embodiment of the method, the predicting step encompasses using the first characteristic determined to predict probabilities of a plurality of cells from the embryo having a plurality of characteristics. In an embodiment of the present disclosure, characterizing an embryo includes characterizing the embryo based on all of the predicted probabilities associated with each determined one or more characteristics. An embodiment of the present disclosure further comprises repeating the determining, predicting and characterizing steps for a plurality of embryos. In an embodiment, the determining step includes using an informatics based method to determine the first characteristic, such as the PARENTAL SUPPORT™ method.

In an embodiment, the at least one characteristic may include at least one genetic condition. In an embodiment, the first characteristic may include a ploidy state. In an embodiment, the first characteristic may be one of euploid or aneuploid. In an embodiment, the at least one characteristic includes at least one of: (i) a ploidy state; (ii) any trisomies being UCA or MCA; (iii) parental origin of any aneuploidy; (iv) a presence or absence of a disease linked gene; (v) a count of any aneuploid chromosomes; (vi) a chromosomal identity of any aneuploid chromosomes; and (vii) any other genetic condition. In an embodiment, the first at least one characteristic is defined by one or more subcharacteristics.

In one embodiment of the present disclosure, the characterizing step includes grouping the embryo into a group defined by at least one characteristic, wherein each group contains zero, one or more embryos, and any embryos within a particular group share at least one characteristic. In an embodiment of the present disclosure, the characterizing step includes ranking the embryo based on an estimated likelihood of that embryo developing as desired. In an embodiment of the present disclosure, the ranking of embryos is performed to select at least one embryo to insert into a uterus. In an embodiment, a Monte-Carlo simulation is used to predict the probability of the second cell.

In an embodiment of the present disclosure, the subcharacteristic may include at least one of (i) aneuploid, mosaic or euploid; (ii) UCA trisomy or MCA trisomy; (iii) maternal or paternal; (iv) present or absent; (v) one, two, three, four, five, six, seven, eight, nine, ten, eleven, twelve, thirteen, fourteen, fifteen, sixteen, seventeen, eighteen, nineteen, twenty, twenty-one, twenty-two, twenty-three or twenty-four; (vi) chromosome one, chromosome two, chromosome three, chromosome four, chromosome five, chromosome six, chromosome seven, chromosome eight, chromosome nine, chromosome ten, chromosome eleven, chromosome twelve, chromosome thirteen, chromosome fourteen, chromosome fifteen, chromosome sixteen, chromosome seventeen, chromosome eighteen, chromosome nineteen, chromosome twenty, chromosome twenty-one, chromosome twenty-two, X chromosome or Y chromosome; (vii) Aneuploidy, Breast cancer (BRCA1), Congenital Adrenal Hyperplasia, Cystic Fibrosis, Duchenne Muscular Dystrophy, Familial Adenomatous polyposis coli (FAP), Familial Alzheimer's disease, Fragile X, Hemophilia, Huntingtons Disease, Klienfelters Syndrome, Marfans Syndrome, Myotonic Dystrophy, Sickle Cell Disease, Spinal Muscular Dystrophy, Tay Sach's Disease, Thalassemia, Translocation, Wiskott-Aldrich syndrome or X-Linked Mental Retardation; and (viii) nullsomy, monosomy, disomy, trisomy, or tetrasomy.

In an embodiment, an embryo may be excluded a priori from consideration of insertion into a uterus due to a prediction, in at least one cell of the embryo, of at least one of: (i) a viable trisomy; (ii) a viable uniparental disomy; (iii) any other chromosomal abnormality; (iv) an undesired disease linked gene; and (v) poor physical characteristics of an embryo.

Embryos that are euploid are typically considered most likely to develop as desired; embryos that are mosaic may be considered less likely to develop as desired, and embryos that are aneuploid may be considered the least likely to develop as desired. An embodiment may use the determined ploidy state of one or more cells from an embryo, along with a model of how mosaicism arises, to determine the likely ploidy states of the untested cells in an embryo. In this embodiment, the determined ploidy state of the measured cells may be used to predict the fraction of remaining, untested cells that are euploid, and therefore the likelihood that a given embryo will develop as desired if transferred to a receptive uterus. Another embodiment of the present disclosure may use the determined ploidy state of one or more cells from an embryo, in combination with empirical embryo development data to predict the probability of the ploidy state of the untested cells. In the above embodiments, the information generated above on the tested and untested cells may be used to determine the likelihood that a given embryo will develop as desired if transferred to a receptive uterus.

### Calculating Aneuploidy Type Probabilities

In one embodiment of the present disclosure, the type of aneuploidy measured in cell(s) taken from an embryo may be used to determine the relative likelihood that some or all of the remaining cells in the embryo are euploid. This determination is based in part on the fact that UCAs are indicative of meiotic errors and MCAs are indicative of mitotic errors, and that embryos containing cells with meiotic stage errors are less likely to contain euploid cells than embryos that have one or more cell with a mitotic state error. Additionally, it is assumed that embryos completely made up of aneuploid cells are less likely to develop as desired than those containing euploid or mosaic cells. Given the nature of the various disjunction errors, may be assumed that embryos with measured UCAs are less likely to develop as desired than embryos with measured MCAs. In the case of uniparental disomies (UPD) and tetrasomies it is possible to conduct a similar analysis to determine whether the observed aneuploidy is more likely due to a meiotic error or whether the observed aneuploidy is due to a mitotic error. In an embodiment of the present disclosure, it may be assumed that the chance of euploid cells after a mitotic error has occurred is greater than the chance of euploid cells after a meiotic error has occurred.

In one embodiment of the present disclosure, the probability that a given embryo that tested aneuploid at one or more cells may contain some euploid cells may be calculated. The probability that an untested cell taken from the an embryo in which one or more cells is tested is euploid is designated P(E). In an embodiment, P(E) may be estimated using the probability of each of the trajectories t₁, i=1 . . . T in FIG. 13 that could have given rise to the measured copy number on each chromosome.

In one embodiment of the present disclosure, the present method may be used to estimate of the probability P(E) for one chromosome at a time. In order to estimate this probability, P(E) may be calculated as follows:

$$P ( E \vert M ) = {\sum }_{i = 1 … T} P ( E \vert {t}_{i} ) P ( {t}_{i} \vert M )$$

wherein M is the measurement of a chromosome copy number, P(E|tᵢ) is the probability that another cell in the embryo is diploid on the chromosome of interest, given trajectory tᵢ and P(tᵢ|M) denotes the probability of the trajectory t, given the measurement M This may be computed as follows:

$$P ( {t}_{i} \vert M ) = \frac{P ( M \vert {t}_{i} ) P ( {t}_{i} )}{P ( M )}$$

P(M|tᵢ)=1 if tᵢ is a trajectory that results in that measured number of chromosomes, M, and 0 otherwise. Hence, for relevant trajectories, it may be assumed that P(tᵢ|M)=P(tᵢ)/P(M), which can be computed from FIG. 13 by looking at the probability of trajectory tᵢ over all possible trajectories that give rise to measurement M.

$$P ( {t}_{i} \vert M ) = \frac{P ( {t}_{i} )}{{Σ}_{i s . t . trajectory ti generates M} P ( {t}_{i} )}$$

In one embodiment, the probability that another cell in the embryo is disomic at that chromosome P(E|tᵢ), may be computed, given that the biopsied cells followed trajectory tᵢ. This may be computed either in closed form or by a method such as a Monte-Carlo method where the replication and division of the chromosomes from one cell to the 8 cell stage is simulated. In one embodiment, it may be assumed that one cell is forced to follow trajectory tᵢ, and P(E|tᵢ) may be calculated by simply counting the number of other cells that are euploid on that chromosome over many simulations. In an embodiment, other mathematical or computer based methods may be used as applicable, and any number of divisions may be modeled. In an embodiment, two or three divisions may be modeled. In an embodiment, four, five, six, seven or more divisions may be modeled.

In an embodiment of the present disclosure, a method is given here to estimate P(E_{c}), for multiple chromosomes, where P(E_{c}) denotes the probability that a cell in the embryo is euploid on chromosome c, c=1 . . . 24. This embodiment may use the method for estimating P(E) for an individual chromosome, described above, and repeating it for all chromosomes. In an embodiment, one may compute P(E_{c}|M) to rank the embryos. Assuming that all chromosomes are independent, one may estimate the probability that a particular embryonic cell is euploid in all chromosomes as:

P(euploid on all chromosomes)=Π_{c}P(E_{c}|M_{c})

In one embodiment of the present disclosure, P(E_{c}) may be calculated as above for a subset of the 24 chromosomes by simply taking c to be the desired number between 2 and 23. In another embodiment of the present disclosure the expected number of euploid cells in an embryo may be computed with a set number, N, of cells before biopsy as follows:

Expected Euploid Cells=(N−1)P(euploid on all chromosomes).

In another embodiment of the present disclosure, the probability that another cell taken from the same embryo is euploid may be calculated, P(E), after the biopsy and analysis of a plurality of blastomeres.

To do this, let M_{c1} and M_{c2} represent the measurement on chromosome c in cells 1 and 2. In one embodiment, P(E|M_{c1},M_{c2}) may be calculated in closed form. In one embodiment, P(E|M_{c1},M_{c2}) may be computed by Monte-Carlo simulation of the model. In one embodiment, P(E|M_{c1},M_{c2}) may be calculated by simulating multiple three stage divisions as above, and for all cases that result in two cells with respective measurements M_{c1} and M_{c2}, find the fraction of the other cells in the embryo with a disomic chromosome c.

In another embodiment of the present disclosure, the probabilities p₁, p₂ and p₃, i.e., the scenario in which three mitotic events occur, may be calculated on a per-sample basis rather than aggregated over multiple samples. In this embodiment, the ratios r₁₂=p₁/p₂ and r₂₃=p₂/p₃ may be calculated from the aggregated data, as described above, using the assumption that this ratio stays roughly the same from one sample to another. This embodiment may use the estimate p₁ for each sample, which may be simplified as p, and M denotes the set of measurements on all chromosomes in a cell: M={M_{c}}, c=1 . . . 24. In this embodiment, p may be calculated using maximum a posteriori probability and Bayes Rule:

$$p = arg {max}_{p} P ( p \vert M ) = arg {max}_{p} \frac{P ( M \vert p ) P ( p )}{P ( M )}$$

In some embodiments, it is possible to maximize over p one may drop the denominator P(M), and P(p) may be computed from the aggregated data over multiple embryos. In an embodiment, each of the measurements M_{c} may be treated as conditionally independent given p, hence we find p from:

p=arg maxₚΠ_{c}P(M_{c}|p)P(p)

where P(M_{c}|p) is straightforward to compute based on simulation or in closed form from FIG. 13. This embodiment, may be extended to the two cell biopsy case, in which the ploidy state may be measured on all chromosomes on both cells M={M_{1,c}M_{2,c}}, c=1 . . . 24 and the determination of p may be written as:

p=argmaxₚΠ_{c}P(M_{1,c},M_{2,c}|p)P(p)

where P(M_{1,c},M_{2,c}|p) may be found by simulation. In this embodiment, the resultant value of p may be used to compute P (euploid on all chromosomes) as described above, which may be then used to rank embryos.

In another embodiment, one could use a similar approach to compute the probability that at least one cell is euploid at that chromosome. In an embodiment, the above calculations may be used to determine whether at least 25%, at least 50%, at least 75% or at least 100% of the cells are euploid at that chromosome. In an embodiment, P(N_{ec}|M_{c}) may also be directly estimated by Monte-Carlo, or other computer based simulation, rather than breaking it down into the constituent terms.

In an embodiment, the probability calculations above account for the assumption that the number of cells with various types of aneuploidy in a cell may change as the embryo develops, and the probability that an embryo will develop as desired may depend partly on the number and ploidy state of those cells.

In one embodiment of the present disclosure, cells with aneuploidy on a preselected set of chromosomes, for example trisomy 8, 13, 21, X and/or Y, may be eliminated from consideration for implantation a priori. In another embodiment, other sets of ploidy states on other chromosomes may be used for a priori selection.

In another embodiment, a model of mosaicism which allows for chromosomes to be lost may be used. In the embodiment described above, an assumption is made that the two post-division cells contain, between them, both of the copies of a chromosome that divides during mitosis, either equally (1,1) or in an imbalanced fashion due to mitotic non-disjunction (0,2) or (2,0). In this embodiment, a model may be used that allows for the possibility that a chromosome is completely lost during disjunction so that the state of the chromosome in the post-division may be any of the following: 1,1 or 0,2 or 2,0 or 1,0 or 0,1 or 0,0. In another embodiment of the present disclosure, a model may be used that assumes that other possibilities may occur upon cell division, such as extra copies of a chromosome being produced.

In an embodiment, data from Hapmap or similar data concerning crossover likelihoods during meiosis, may be used to determine the probability that a non-disjunction error occurred during meiosis to give rise to a UCA or an MCA. In this embodiment, an informatics based approach, such as PARENTAL SUPPORT™, may be used to take advantage of crossover probabilities, and may phase the genetic data of the blastomere. In this embodiment, one may identify chromosomes that have matching crossovers, or other characteristics that indicate that the non-disjunction error occurred during meiosis, and make that determination for each chromosome.

### Embryo Ranking

The general concept behind embryo ranking is to categorize embryos into groups or bins that have different probabilities of developing normally, and then to rank the embryos by those relative probabilities. In one embodiment of the present disclosure, the ranking may be used to decide which embryo(s) to transfer in the context of IVF. In one embodiment, the first step is to differentiate embryos into groups and then calculate the probability that the embryos in each of the bins have to develop as desired. In an embodiment, the relative probability of an embryo to develop as desired may be calculated, using contingency tables, using published embryo development data, using other sources of empirical embryo development data, using a combination of various sources of embryo development data, or using embryo development theories. In an embodiment, those probabilities can then be used to determine which embryo(s) to transfer in the context of IVF; this may be done by selecting the embryo whose calculated probability of developing normally is the greatest. Many of the embodiments described herein focus on methods of differentiating the embryos using bins related to particular ploidy states. Some examples of the types of ploidy states that may be used to categorize the embryos include MCA trisomy and UCA trisomy, the parental origin of any aneuploid chromosomes, the number of aneuploid chromosomes observed, the identity of aneuploid chromosomes, or some combination thereof. The embryos may also be differentiated using other physical characteristics, for example, embryo morphology, embryo size, or the absence or presence of certain genotypes.

In one embodiment, the first step may be to decide on a set of groups, or bins, and a method that may be used to divide the embryos into those groups. Each bin may be defined by a set number of characteristics that are each associated with a probability of normal embryo development. In this embodiment, the next step may be to determine the probabilities that the embryos in each of those groups is likely to develop as desired. In this embodiment, the probabilities may be determined using empirical data and calculating those probabilities, or by other methods described elsewhere in this document.

The number of bins may be very small, for example two, or the number of bins may be very large, such that after categorization, only a small percentage of the bins are populated, or the number may be anywhere in between. Any number of bins may be used. In one embodiment, a large number of bins may be used so that each embryo may be differentiated from every other, and the ranking will be more specific. In some embodiments, some of the bins may have essentially equal probabilities associated with them. In an embodiment, a small number of bins may be used so that the calculation of the likelihood that embryos in a given bin have to develop as desired is based on a limited amount of empirical embryo development data. The fewer the bins, the more empirical data will be available for each bin, and thus the more accurate the prediction may be.

In one embodiment, subcharacteristics, such as basic ploidy states may be used as bins: nullsomy, monosomy, disomy and trisomy. In another embodiment, the trisomic bin may be separated into MCA trisomies, and UCA trisomies. In another embodiment, each chromosome may be considered separately, so that, for example, if each chromosome is categorized into five bins, then 5²³ bins would be used. Some bins may contain no embryos. In some embodiments the bins may reflect the possibility of the ploidy state being known for more than one cells from an embryo, and that those ploidy determinations may or may not correspond. In some embodiments, two or three bins may be used. In some embodiments five to ten bins may be used. In some embodiments, ten to one hundred bins may be used. In some embodiments, one hundred to one million bins may be used. In another embodiment, one could train more fine grained probabilities than just the P(D/t), P(D/m), P(D/n). In one embodiment, embryos may be ranked based on more complex abnormalities, for example, a combination of a monosomy and a trisomy, or two trisomies.

### Distinguishing Meiosis I/II Errors and Mitosis Errors

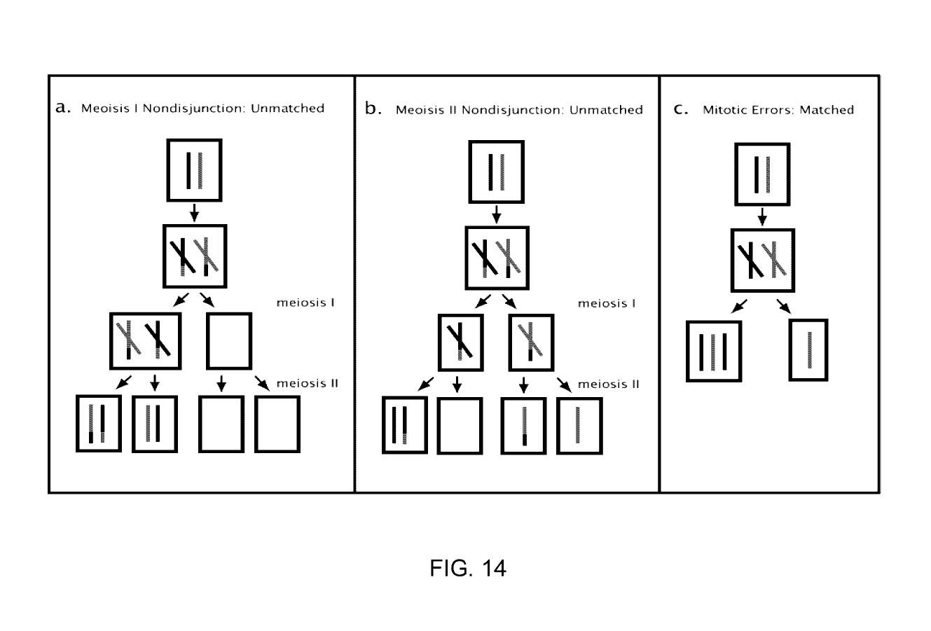

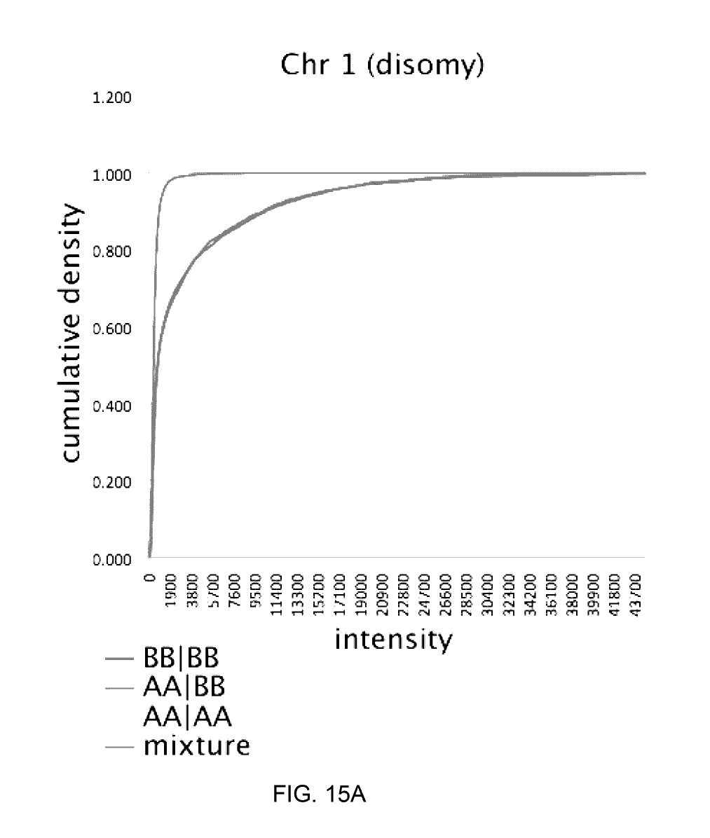

In one embodiment of the present disclosure, the embryos may be differentiated by the type of non-disjunction error. For example, they may be differentiated by errors that most likely occurred during meiosis, and those that likely occurred during mitosis. Matched errors, (MCA) where two of the three chromosomes of a trisomy are identical, will generally indicate mitotic errors; unmatched errors, (UCA) where all three homologues of a trisomic pair of chromosomes are different, will generally indicate that recombination likely occurred in meiosis I between homologous chromosomes to create a tetratype chromosome state. This concept is illustrated in FIG. 14. In an embodiment, the method illustrated in FIG. 14 may be used to determine the type of non-disjunction errors. In an embodiment, other methods may be used to decipher the type of non-disjunction errors. In one embodiment of the present disclosure, one may use a method that uses the parental genotype contexts or parental haplotypes. In one embodiment, a partial or full delineation of parental haplotypes is made, and those haplotypes, along with the measured genetic information from the blastomere, and an informatics method such as PARENTAL SUPPORT™ are used to help determine the ploidy state of the blastomere.

Parental contexts can be highly informative when attempting to determine the embryonic chromosome state. The parental context for a given SNP is the identity of the two corresponding SNPs on both the mother and the father, representing the set of possible SNP identities from which the embryo genotype originates. According to the mechanism of meiosis, in the case of a normal euploid embryo, at a given locus, one SNP will be maternal in origin, and the corresponding SNP on the homologous chromosome will be paternal in origin. The identity of the SNP of maternal origin will be that of one of the two maternal SNPs at that locus, and the identity of the SNP of paternal origin will be that of one of the two paternal SNPs at that locus. The parental context for a given SNP may be written as “m₁m₂|p₁p₂”, where m₁ and m₂ are the genetic state of the given SNP on the two maternal chromosomes, and the p₁ and p₂ are the genetic state of the given SNP on the two paternal chromosomes. The genotype at a given SNP of a euploid embryo with the parental context of m₁m₂|p₁p₂ could be m₁,p₁, m₁,p₂, m₂,p₁ or m₂,p₂.

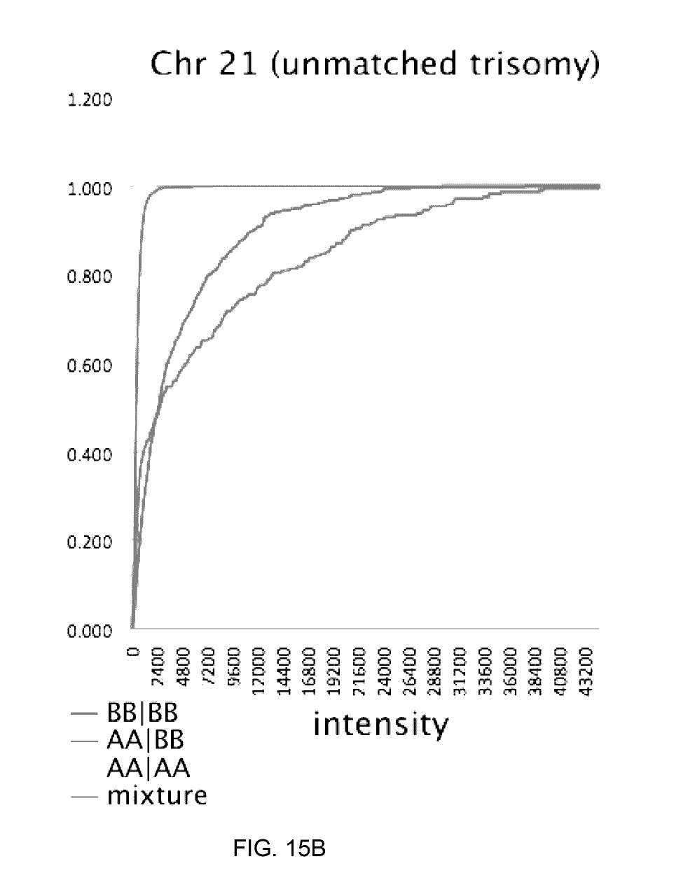

In one embodiment of the present disclosure, the matched/unmatched discrimination algorithm may use the parental contexts. This embodiment may use a method to determine the difference in the distribution of measured embryonic SNPs between the different parental contexts under matched and unmatched errors. This embodiment is illustrated in FIGS. 15A-15B. The distribution of measured embryonic SNPs in the heterozygous context is expected to be different for different ploidy states, and when the distributions are considered for all of the contexts, each different embryonic ploidy state has its own characteristic set of distributions. Typically, heterozygosity increases under unmatched errors but stays constant under matched errors.

For example, suppose that loci are randomly selected from the AA|BB and BB|BB contexts on the A microarray detection channel. Under maternal trisomy caused by a MCA, the distribution of AB|BB should look like a bimodal mixture of the loci randomly selected from AA|BB and BB|BB. To illustrate this example, subdivide the A and B contexts each into four subcontexts: A₁ and B₁ are alleles from chromosome copy 1, and A₂ and B₂ are from chromosome copy 2. A matched error consistently results in loci that are A₁B₂B₂|BB and A₂A₂B₁|BB, which results in a context distribution no different than a random selection from A₁A₂|BB, B₁B₂|BB. In contrast, consider the case where the trisomy is caused by a UCA. With an unmatched copy error, there are two more subcontexts, i.e., A₃ and B₃. This results in 3-factorial (six) types of loci in the AB|BB context: A₁B₂B₃|BB, A₁A₂B₃|BB, A₁A₃B₂|BB, A₂A₃B₁|BB, A₃B₁B₂|BB, and A₂B₁B₃|BB. As a result, AB|BB under unmatched trisomy has a trimodal distribution and does not look like a mixture of the distributions of AA|BB and BB|BB. This is because heterozygosity is higher than expected in the case of unmatched trisomy. Thus, to discriminate matched from unmatched errors, one may formulate the null hypothesis as maternal trisomy caused by a matching error, and then attempt to match the cumulative density function of AB|BB with a mixture of the AA|BB and BB|BB cumulative density functions. Established statistical methods such as the Kolmogorov-Smirnov goodness of fit test may be used to determine a confidence interval, and if the difference between the AA|BB/BB|BB mixture and the actual cumulative distribution function (CDF) of AB|BB is in the rejection region, the null hypothesis may be rejected, and it can be concluded that the trisomy is caused by an unmatched error. This may be done separately for both detection channels (X and Y) on Infinium, or other, microarrays, and then the probability of rejection is combined.

Differentiating Meiotic from Mitotic Errors with Phasing (Sperm Genotyping)

In another embodiment of the present disclosure, a method may be used that includes phasing the embryonic data, and determining which chromosomes or segments of chromosomes in the embryo originate from which parent. This method may be particularly useful, for example, in a case where, due to crossover(s) during meiosis, limited exchange of genetic material between homologous chromosomes results in a tetratype where sister chromatids are mostly identical. Although phasing is a challenging problem, methods have been described elsewhere, such as the PARENTAL SUPPORT method, that are specifically designed to phase noisy unordered single cell genotype measurements. It is possible to use this capability to differentiate meiotic (UCA) from mitotic (MCA) errors.

In an embodiment of the present disclosure, the present method is used in conjunction with PARENTAL SUPPORT™ and may, assume disomy but may also consider the possibility of trisomy in its theoretical derivation. In this embodiment, for each chromosome, on n SNPs data D=(D₁, . . . , Dₙ) is generated where data on i^{th} SNP consists of (X,Y) channel data for all k blastomeres, l sperm cells, mother genomic and father genomic, i.e. Dᵢ=(D^{e}ᵢ,D^{s}ᵢ,D^{m}ᵢ,D^{f}ᵢ), where D^{e}ᵢ=(X^{e}ᵢ₁,Y^{e}ᵢ₁), . . . , (X^{e}ᵢₖ,Y^{e}ᵢₖ)), D^{s}ᵢ=((X^{s}ᵢ₁,Y^{s}ᵢ₁), . . . , (X^{s}ᵢₗ,Y^{s}ᵢₗ)), D^{m}ᵢ=(X^{m}ᵢY^{m}ᵢ) D^{f}ᵢ=(X^{f}ᵢ,Y^{f}ᵢ). In this embodiment, for each embryo target, j=1, . . . , k, on each SNP i, the goal is to derive the most likely allele call g^{j}ᵢ=(n^{A}ᵢⱼ,n^{B}ᵢⱼ), by calculating P(gᵢⱼ|D) for all possible allele values, returning the value with highest probability, and returning that probability as the confidence in that call. In this embodiment, by first calling the copy number classification algorithm, it is possible to derive the copy number hypothesis likelihood given the data P(fⱼ|D,j)=P(copy number hypothesis=fⱼ on jth target|D). For SNP i, on blastomere j:

P(gᵢⱼ=D)=Σ_{F=(f1 . . . fk)}P(gᵢⱼ|F,D)(Π_{t=1 . . . k}P(fₜ|D,t))

where F is the set of copy number hypotheses for all blastomeres. The sum over F=(f₁ . . . . . fk) represents the sum over all possible combinations of hypotheses over all embryo targets 1 . . . k, P(g^{ij}|P,D) is the conditional probability of the allele call gᵢⱼ assuming a particular set of copy number hypotheses (F) over all blastomeres given the data. It is possible to derive this probability for any value of F, which may include trisomies on particular blastomeres, and to analyze the hypotheses in a set F since the probability of each hypothesis on each blastomere is dependent on the probabilities of the hypotheses on the other blastomeres. If two haplotypes are most likely in a trisomic state, the chromosome may be called matched, and if the hypothesis of three haplotypes is most likely, the chromosomes may be called unmatched. Because the haplotyping method specifically orders the genotype measurements into haplotypes, it may achieve higher sensitivity than some methods.

### Analyzing Polar Bodies and Multiple Single Cells Simultaneously

In another embodiment, polar bodies and/or other cells may be a source of extra information from which embryos can be ranked. In an embodiment, any source of genetic information that correlates with the ploidy state of the embryo can be used, for example, additional cells taken from or originating from the embryo, including polar bodies or any other appropriate source. In an embodiment, the genetic information is gathered from two cells of a 3-day embryo. In another embodiment, the genetic information is gathered from two or more cells from a 5-day embryo. In any of the above embodiments, the additional genetic data is used to validate the prediction of a “normal” embryo based on the scoring scheme. In any of these embodiments, various sets of data can be combined to make increasingly accurate predictions of the actual genetic state of the embryo. In any of these embodiments, the additional genetic information may improve the chance of correctly deducing the ploidy state of the remaining cells in the embryo.

In one embodiment of the present disclosure, the probabilities (e.g. P(D/t1)) may be computed on a per chromosome basis. In another embodiment, this method may be executed on each chromosome segment; that is segment by segment. For example, in a case where low confidences are caused by de novo mitotic translocations, this could be caused by embryos in which one blastomere has a trisomy on a tip and another blastomere has a monosomy on the corresponding tip. This embodiment of the method takes into account unbalanced translocations, and may give more accurate results when said translocations occur at a significant level.

In one embodiment of the present disclosure, the embryos may be grouped based on the parental origin of the chromosomes in the cell. For example, some studies indicate that if a trisomy is detected at a given chromosome on a blastomere, the likelihood that the embryo from which the blastomere was biopsied contains euploid cells is higher if two of the three trisomic chromosomes originate from the father, as opposed to if two of the three trisomic chromosomes originate from the mother. In an embodiment, the parental origin of chromosomes in the case of a uniparental disomy, or a monosomy may be used to categorize the embryos. In this embodiment, if a blastomere is measured to have a paternal monosomy, one would expect an increased likelihood of another cell in the embryo containing a maternal MCA trisomy.

In another embodiment, one may use the number of MCAs in a single cell in order to rank the embryo. In this embodiment, if a cell is determined to have MCAs measured at more than one chromosome, is the embryo would be considered to be less likely to contain euploid cells than an embryo from which one blastomere has been determined to have MCAs measured at only one chromosome. In another embodiment of the present disclosure, different combinations of aneuploidy types at different chromosomes, as measured on a blastomere from that embryo, may be used to categorize the embryos. In another embodiment of the present disclosure, the chromosomal identity of MCAs, or other ploidy states, may be used to rank the embryos. For example, data may show that embryos with an MCA measured at chromosome 3 may be more likely to develop as desired than embryos with an MCA measured at chromosome 6. In another example, a paternal trisomy at chromosome 9 may be considered more likely to develop as desired than a maternal trisomy at chromosome 9. In another example, a monosomy at chromosome 4 may be more likely to develop as desired than a monosomy at chromosome 2.

In another embodiment of the present disclosure, embryos may be differentiated into bins based on properties other than types of aneuploidy. For example, embryos may be differentiated based on the presence or absence of any alleles known to be correlated with implantation and/or the health of a baby. In one embodiment, embryos may be differentiated into bins based on physical characteristics, such as morphology, size, shape, color, transparency, or the presence or absence of various features. In some embodiments of the present disclosure, embryos may be differentiated based on a combination of qualities, such as those listed here. For example, embryos may be differentiated based on ploidy state and morphology; embryos may be differentiated based on ploidy state and the presence of an implantation related alleles; embryos may be ranked based on ploidy state and the parental origin of any trisomies.

In one embodiment of the present disclosure, the embryos are biopsied at day 5 from the tropechtoderm. Trophectoderm biopsy is a newer approach to PGD that assesses the chromosomal status of the trophectoderm immediately prior to implantation. In contrast with single cell biopsies at the 3 day stage, the trophectoderm biopsy typically yields between 4-10 cells. In one embodiment of the present disclosure, the biopsied cells are genotyped together. In this embodiment, the genotyping results may need to be interpreted using non-standard methods. In some embodiments, the tropechtoderm sample may consist of a mosaic population of cells. In this embodiment, the present method may be used in combination with an informatics based methods such as the PARENTAL SUPPORT™ algorithm to choose the optimal hypothesis among a set of hypotheses that describe the various possible states of mosaic aneuploidy in the trophectoderm. In another embodiment of the present disclosure, the individual cells from the tropechtoderm biopsy are separated, and the ploidy state of one or more of them are called individually. In one embodiment, one or two cells may be biopsied from the embryo. In one embodiment, three to ten cells may be biopsied. In one embodiment, eleven to twenty cells may be biopsied. In one embodiment, more than twenty cells may be biopsied. In one embodiment, an unknown number of cells may be biopsied. In one embodiment, the cells may be biopsied at day 2 or day 3. In one embodiment, the cells may be biopsied at day 4, 5 or 6. In one embodiment, the cells may be biopsied later than day 6.

### ADDITIONAL EXPERIMENTAL SECTION

In one embodiment of the present disclosure, the method was implemented as follows: once the IVF cycle commenced on Day 0 (when harvested eggs had undergone fertilization), the clinic alerted the lab as to the number of fertilized eggs. The embryos underwent morphological evaluation during their development in vitro, and embryos of good morphological quality on Day 3 underwent a single blastomere biopsy for PGD according to standard IVF protocols. The IVF laboratory cultured the embryos to the blastocyst stage using sequential, stage-specific culture media and an advanced, ultra-stable, low-oxygen culture system that is able to adapt to the changing metabolism of the blastulating embryos. The IVF centers then shipped the blastomeres on ice by courier, and the lab received the samples on the morning of Day 4.

Single cells were manually isolated using a micromanipulator (Transferman NK2-Eppendorf). All single cells were washed sequentially in three drops of hypotonic buffer (5.6 mg/ml KCl, 6 mg/ml bovine serum albumin) to reduce the possibility of contamination. Three different lysis/amplification protocols have been used in the analysis: (i) Multiple Displacement Amplification (MDA, GE Healthcare, Piscataway, N.J.) with Alkaline Lysis Buffer (ALB), (ii) Sigma Single Cell Amplification Kit (WGA, Sigma, St. Louis, Mo., USA) with Sigma Proteinase K Buffer (Sigma PKB), (iii) and MDA with Proteinase K Buffer (PKB). In protocol (i) cells were frozen at −20° C. in ALB (200 mM KOH, 50 mM dTT) for 30 minutes, thawed, and neutralized with an acid buffer (900 mM Tris-HCl, pH 8.3, 300 mM KCl, 200 mM HCl). Protocol (ii) was performed according to the manufacturer's instructions. For protocol (iii), cells were placed in PKB (Arcturus PICOPURE Lysis Buffer, 50 mM DTT), incubated at 56° C. for one hour, and then heat inactivated at 95° C. for ten minutes. For protocols (i) and (iii), MDA reactions were incubated at 30° C. for 2.5 hours and then 95° C. for five minutes. Genomic DNA from bulk tissue (Epicentre MASTERAMP Buccal Swabs, Madison, Wis., USA) was isolated using the DNEASY Blood and Tissue Kit (Qiagen, Hilden, Germany). No template controls (hypotonic buffer blanks) were performed for each amplification method.

Both amplified single cells and bulk parental tissue were genotyped using the Illumina (San Diego, Calif., USA) INFINIUM II genome-wide genotyping microarrays (HapMap CNV370DUO or CNV370QUAD chips). For the bulk tissue, the standard Infinium II protocol (www.illumina.com) was used and required call rates of >97% using standard BEADSTUDIO allele calling. Single cells were genotyped using a modified Infinium II genotyping protocol, such that the entire protocol, from single cell lysis through array scanning, was completed in fewer than 24 hours. A variety of time saving modifications were made to the protocol, for example, the duration of the amplification and hybridization steps were reduced by 50% and 63%, respectively. Samples and analytes were tracked using a laboratory information management system (LIMS). Raw data were parsed and used as input for ploidy state analysis.

Upon completing the genotyping assays, the PARENTAL SUPPORT™ method was used to determine the ploidy state of each of the chromosomes in each embryo, including whether any detected trisomies were MCAs or UCAs, and the parental origin of the chromosomes. Each of the 23 chromosomes from the embryos were then categorized into five bins: (1) euploid, (2) one monosomic chromosome, (3) one trisomic chromosome (4) one nullsomic chromosome and (5) other aneuploidy, for a total of 5²³ bins, many of which were statistically treated the same. Embryos whose biopsied blastomere was euploid were considered to be the most likely to implant, and in the cases where euploid embryos were available, those were transferred. A number of aneuploidy states were rejected a priori, these include: trisomy 8, 9, 13, 16, 18, 21, 22 and 23, as well as paternal UPD 6, 11, maternal UPD 7, and any UPD at 14, 15 or 23. Nine embryos that were determined to be aneuploid and were ranked were transferred, along with one euploid embryo, in six IVF cycles. Of those cycles, one pregnancy results. The transferred aneuploid embryos had the following aneuploidy states: (1) monosomy 16, (2) trisomy 16, (3) monosomy 22, (4) monosomy 14, (5) trisomy 15+monosomy 8, 10, 22, (6) monosomy 19, (7) monosomy 16, (8) trisomy 14, and (9) monosomy 1+trisomy 9.

### Statistical Demonstration of the Method

A set of virtual embryos were assembled, a virtual blastomere was biopsied from each embryo, and the ploidy state was determined. The embryo ranking method was then used to rank the embryos, and the rate of expected implantation using the embryo ranking method was compared to the expected implantation when embryos were selected randomly. The ploidy state distributions of the virtual embryos were determined using empirically measured data from both internal and published studies, and the calculated relative probabilities that the embryos have to develop as desired were estimated based on empirical embryo development data.

Data from two published studies, in which 112 embryos were studied both on Day 3 and Day 5 for chromosome copy number using fluorescent in situ hybridization (FISH) technology, (Baart et al., Hum. Reprod., 2006, Vol 21(1), p. 223-233; and Baart et al., Hum Reprod., 2004, Vol 19(3), p. 685-693) were analyzed to create different groups, and determine the relative development probabilities. Note that the data from these studies was performed with FISH, only 8 chromosomes per cell were analyzed and the ploidy calling on these chromosomes may be expected to have a high error rate. The results were analyzed in order to convert the data into a computable format where each embryo has 205 features. The features were clustered into 2 groups: (1) features at Day 3 such as number of copies of each chromosome, the concordance between results when two cells are analyzed from each embryo, and summary features such as the total number of nullsomies, monosomies, and trisomies observed in each cell; and (2) features at Day 5 such as the percentage of cells that have 0, 1, 2, 3 or 4 copies of each chromosome over the 8 chromosomes measured; the clinical diagnosis at Day 5 of normal or abnormal; and the growth state of the embryos as determined by the number of cells on Day 5 and whether arrested or not.

The Day 3 features were analyzed and the embryos were scored for the likelihood of being euploid on Day 5 after a particular abnormality was observed in one or two biopsied blastomeres on Day 3. The Day 5 features were used as the key outcomes to be modeled and the inputs to the model were the measurements on Day 3. The model was trained using the probability P(D) of embryos in the training dataset being euploid (disomic on the relevant chromosomes across more than 80% of cells analyzed in the blastocyst) on Day 5 after a chromosome was found to be either (1) trisomic in one biopsied cell on Day 3 (P(D/t₁)), (2) trisomic in both biopsied cells on Day 3 (P(D/t₂)), (3) monosomic in one biopsied cell on Day 3 (P(D/m₁)), (4) monosomic in both biopsied cells on Day 3 (P(D/m₂)), (5) nullsomic in one biopsied cell on Day 3 (P(D/n₁)), or (6) nullsomic on both biopsied cells on Day 3 (P(D/n₂)) as described below. Leave-one-out training was used, i.e., the embryo to be scored was left out while the algorithm learned these probabilities. Other methods of training predictive algorithms are well known in the literature, and may equally well be used here. Two alternate approaches were used to learn the probabilities P(D/t₁) . . . P(D/n₂): (1) by ignoring chromosome identity (e.g. chromosome 1, 22, X, etc) and pooling the results over all chromosomes to determine these six probabilities; and (2) in a chromosome specific manner where the probabilities P(D/t₁) . . . P(D/n₂) were learned on a per chromosome basis so that a total of 6×8=48 probabilities were learned. Considered first is the non-chromosome specific model. For the embryo to be scored, the number of chromosomes that were (1) trisomic in one biopsied cell on Day 3 (giving count cu), (2) trisomic in both biopsied cells (cₜ₂), (3) monosomic in one cell (cₘ₁), (4) monosomic in both cells (cₘ₂), (5) nullisomic in one cell (cₙ₁), and (6) nullisomic in both cells (cₙ₂) were counted. The counts cₜ₁, cₜ₂, cₘ₁, cₘ₂, cₙ₁, and cₙ₂ were used for each embryo and a score, S, was computed for that embryo using the model:

S=(P(D|t₁))^{c}^{ₜ₁}(P(D|t₂))^{c}^{ₜ₂}(P(D|m₁))^{c}^{ₘ₁}(P(D|m₂))^{c}^{ₘ₂}(P(D|n₁))^{c}^{ₙ₁}(P(D|n₂))^{c}^{ₙ₂}

The score S represents the probability that an embryo will be euploid on more than a threshold percentage of cells on Day 5 (for the purposes of the training discussed herein, 80% was used as a threshold) for all chromosomes measured, given the observed counts on Day 3, the learned probabilities from the training dataset, and the simplifying assumption that any chromosomes measured disomic on Day 3 will also be disomic on Day 5. In the case where the probabilities are learned on a chromosome specific manner, the algorithm is similar, except that state of each chromosome is evaluated on Day 3 separately. In this case the state of each chromosomes, of index i, is described the values c_{t1,i}, c_{t2,i}, c_{m1,i}, c_{m2,i}, c_{n1,i}, c_{n2,i} where only one these values is 1, corresponding to the state of the chromosome, and the others are 0. The chromosome specific scores were then combined as follows:

$$S = {\prod }_{\text{?}} ( {P}_{i} ( D \vert {t}_{1} ) ) \text{?} ( {P}_{i} ( D \vert {t}_{2} ) ) \text{?} ( {P}_{i} ( D \vert {m}_{1} ) ) \text{?} ( {P}_{i} ( D \vert {m}_{2} ) ) \text{?} ( {P}_{i} ( D \vert {n}_{1} ) ) \text{?} ( {P}_{i} ( D \vert {n}_{2} ) ) \text{?}$$

$$\text{?} \text{indicates text missing or illegible when filed}$$

To demonstrate whether this embryo ranking method has the potential to improve implantation rates, despite the effects of mosaicism, it was determined whether results of a Day 3 biopsy would improve the probability of selecting normal embryos on Day 5. The design of the simulation was to randomly assign the 112 embryos into 14 virtual families with the number of embryos per family ranging from 5 to 12. For each virtual family, either Day 3 embryos were chosen at random or Day 3 embryos were chosen with the highest score S based on the ranking model. It was then determined whether the chosen embryos were euploid on Day 5, and the rate of normal embryos selected with the rate of normal embryos selected on Day 5 was also determined if the embryos were chosen at random, without ploidy data, from the set of embryos that were morphologically normal on Day 5. For the purposes of this evaluation, the assumption was made that the diagnosis of an embryo as “normal” on Day 5 would be highly correlated with successful implantation.

For each virtual family the estimated improvement in the number of normal embryos selected was then calculated under two scenarios: (1) performing a single cell biopsy on Day 3; (2) performing a two-cell biopsy on Day 3. Since the Baart datasets included biopsies of 2 blastomeres, it was possible to emulate a single cell biopsy by leaving one cell out. Note that in the single cell biopsy scenario, the terms P(D/t₂), P(D/m₂), P(D/n₂) and the corresponding counts cₜ₂, cₘ₂, cₙ₂ are all zero and the model becomes simpler. One thousand simulations were performed, involving assigning the embryos to virtual families and estimating the improvement in rate of normal embryo selection. The mean improvement in rates of selecting normal Day 5 embryos using the model of the present disclosure, as compared to using random selection, is shown in Table 6 for both the chromosome-specific model and the non-chromosome specific model.

TABLE 6
The mean improvement in rates of selecting normal Day 5 embryos with
algorithm over random selection.

| | Non - chromosome specific model | | | | | |
|---|---|---|---|---|---|---|
| | | “Implantation | | Chromosome specific model | | |
| | “Implantation | rates” based on | | “Implantation | “Implantation | |
| | rates” based | random | | rates” based on | rates” based on | |
| | on Model | selection | Improvement | Model | random selection | Improvement |
| 1 cell | 42.83 | 27.5 | 55.75% | 44.16 | 28.3 | 56.04% |
| biopsy | | | | | | |
| 2 cell | 47.10 | 27.23 | 72.97% | 48.79 | 27.55 | 77.10% |
| biopsy | | | | | | |

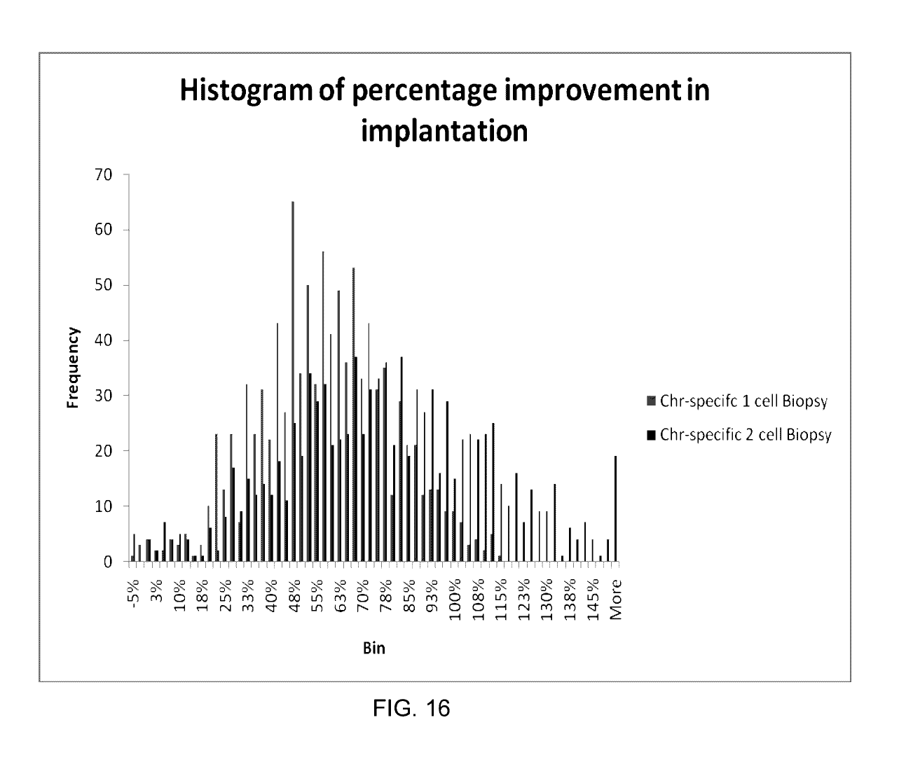

FIG. 16 shows histograms of the improvement in virtual implantation rates for the chromosome specific model and compares the percentage improvement in normal embryo rates on applying the model to a 1-cell biopsy and a 2-cell biopsy. When, using this model system, one cell was biopsied, an improvement of between 50 and 60% in the implantation rates was observed. When two cells per embryo were biopsied, an improvement of between 70% and 80% in the implantation rates was observed.

A similar analysis was performed using data collected internally from donated embryos which had been disaggregated and where the ploidy state for each cell had been determined. In this case, there were no day 5 outcomes, instead, a surrogate was used, in the form of the euploidy status of the remaining cells after the one blastomere has been biopsied. Since it is not known how many euploid cells are necessary for an embryo to develop as desired, the assumption was made that if a certain fraction of cells among the remaining cells are euploid, then that embryo will develop as desired. Several cutoff thresholds were used for the fraction of cells required for the embryo to be considered one that would develop as desired for the purposes of the surrogate outcome. The results are shown in Table 7 where the mean improvement in implantation rates using the model of the present disclosure and internal data, as compared using random selection, is shown. When the threshold was set at 100%, that is, the cell would be considered one which will implant and develop as desired only if 100% of the remaining cells in the virtual embryo are euploid, and only those cells were chosen, then the improvement rate in predicted implantation was 100%. When the threshold was set at 75%, the predicted improvement was 57%; when the threshold was set at 50%, the predicted improvement was 24%; when the threshold was set at 25%, then the predicted improvement was 15%; and when the threshold was that at least one cell in the embryo was euploid, then the predicted improvement was 18%.

TABLE 7
Mean improvement in implantation rates using Model.

| Tresh- | # of | Random | Using | Improve- | |
|---|---|---|---|---|---|
| old | Embryos | Selection | Model | ment | Imp. Range |
| 100% | 3 | 8.33 | 16.67 | 100% | 89-111, SD = 5 |
| 75% | 6 | 21.29 | 33.33 | 57% | 53-61, SD = 2 |
| 50% | 9 | 26.83 | 33.33 | 24% | 21-28, SD = 1 |
| 25% | 13 | 38.02 | 43.93 | 15% | 13-19, SD = 1 |
| 1-cell | 15 | 43.51 | 51.34 | 18% | 15-21, SD = 1 |
| euploid | | | | | |

In another embodiment, a different simulation was run where the model was trained using model parameters from internal day-3 data and the Baart datasets, and the corresponding 5 outcomes were used for validation. In these simulations, an improvement of 55-60% was consistently measured when selecting a highly ranked embryo as compared to a random selection, where a successful implantation was judged as an embryo that was deemed euploid at day 5.

In another embodiment, to address a shortcoming on the Baart datasets, namely that only eight chromosomes were measured using FISH, and that those measurements are error prone (FISH error rates typically run between 10 and 15%), and the embryos were not grouped into relevant families in the published study, a parallel analysis was performed on internally generated data. These data consisted of measured ploidy data taken from disaggregated blastomeres originating from 27 embryos from 8 different families, where the average number of embryos per family was 3.37, and ranged between 1 and 6. The total number of blastomeres analyzed was 110. The minimum number of blastomeres analyzed per embryo was 2 and the maximum number of blastomeres analyzed per embryo was 8. In this analysis, a single-cell biopsy was assumed and a chromosome-specific model was used as described above. In contrast to the previous analysis, only Day 3 data is analyzed: each of the probabilities Pᵢ(D|t₁), Pᵢ(D|m₁), Pᵢ(D|n₁) represent the likelihood that, given a particular state on the biopsied cell (trisomy, monsomy or nullsomy), another cell chosen from the same embryo will be euploid on that chromosome. One implicit assumption was that embryos that contain at least one euploid cell are more likely to self-correct to euploidy by Day 5 than embryos that do not contain any euploid cells. As in other methods described above, a score was assigned to the embryos, except that this score was computed over all 23 chromosomes:

$$S = {{Π}_{i = 1 …23} ( {P}_{i} ( D \vert {t}_{i} ) )}^{{c}_{t 1 , i}} {( {P}_{i} ( D \vert {m}_{1} ) )}^{{c}_{m 1 , i}} {( {P}_{i} ( D \vert {n}_{1} ) )}^{{c}_{n 1 , i}}$$

In this case, the score S represents the probability, given the measurement on the biopsied blastomere, that another blastomere taken from the same embryo would be euploid across all chromosomes. This score was use to rank the embryos for each family and the top scoring embryo for each family was chosen for “implantation”. A Day 3 embryo was considered “normal” if that embryo contained one or more fully euploid cells after the single-cell biopsy. One thousand simulations were run and in each simulation a blastomere was chosen at random from each of the embryos in each of the families. If selected at random, the fraction of embryos that contained at least one normal cell was found to be 44.4%. If selected based on the results of the single biopsied cell, the fraction of normal embryos selected was 78.4%, suggesting an improvement in the rate of selection of normal embryos of 76.3%. Leave-one-out training of the model was used.

In order to evaluate the statistical significance of the result over the 27 embryos, the average score S that an embryo received was based on the computed the score for each blastomere that could be biopsied from that embryo; that was computed for each embryo. From that average score, the 27 embryos were ranked. The sum of the ranks of all of the embryos was then computed and compared to expected sum of the ranks if the embryos were randomly ordered. This canonical statistical technique functioned as a way of determining the statistical significance of a ranking method. It was found that the sum of the rank of the embryos using the Day 3 biopsy was improved as compared to the sum of the random ranks with a p-value of 0.0153.

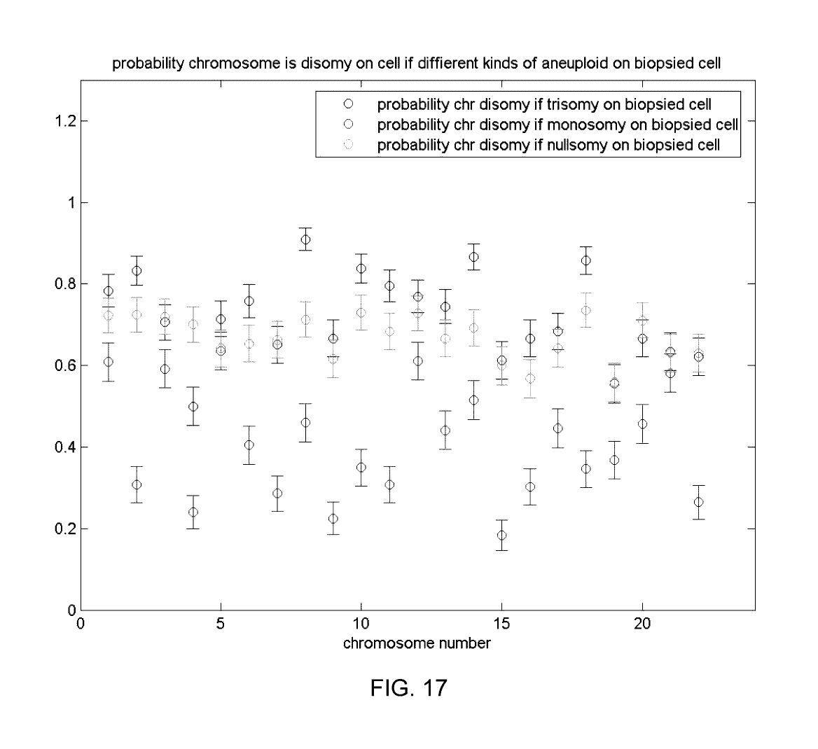

Analysis of the data showed that the improvement in implantation rates is roughly 8% higher when a chromosome-specific model is used. One explanation for this is illustrated in FIG. 17 below where the probabilities Pᵢ(D|t₁), Pᵢ(D|m₁), Pᵢ(D|n₁) for chromosome number i=1 . . . 22 are illustrated. FIG. 17 illustrates the probability of a blastomere in an embryo being diploid on a chromosome if the biopsied cell from that embryo is triploid (blue), monosome (red) or nullisome (green) on that chromosome. The 1-sigma error bar on the estimate of each of these probabilities with limited data is shown. These probabilities vary between chromosomes in a statistically significant manner.

Another example is given here that trains probabilities for 9 bins: trisomy, monosomy, nullisomy: P(D/t), P(D/m), P(D/n); also trisomy of two chromosomes, monosomy of two chromosomes and nullisomy of two chromosomes: P(D/t2), P(D/m2), P(D/n2); and then trisomy+monosomy, trisomy+nullisomy, monosomy+nullisomy: P(D/tm), P(D/tn), P(D/mn). The scoring function (or model) would be:

$$( P ( \frac{D}{t} ) ) \text{?} ( P ( \frac{D}{m} ) ) \text{?} ( P ( \frac{D}{n} ) ) \text{?} x ( P ( \frac{D}{t 2} ) ) \text{?} x ( P ( \frac{D}{m 2} ) ) \text{?} x ( P ( \frac{D}{n 2} ) ) \text{?} x ( P ( \frac{D}{tm} ) ) \text{?} x ( P ( \frac{D}{tn} ) ) \text{?} x ( P ( \frac{D}{mn} ) ) \text{?}$$

$$\text{?} \text{indicates text missing or illegible when filed}$$

Such a model, with a greater number of bins will allow more accurate probabilities to be computed for: (1) how likely that another cell would be euploid if drawn from same embryo; (2) how likely the embryo is to contain normal cells; (3) how likely the embryo is to be normal on day 5.

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A method for identifying an embryo with a highest probability for developing into a euploid individual, from a set of embryos, the method comprising:
obtaining at least one cell from each embryo in the set of embryos;
determining separately for each embryo of the set of embryos, on a computer, a ploidy state of at least one chromosome from the at least one cell, wherein the determining comprises:
measuring genetic data from the embryo and from one or more related individuals, said one or more related individuals comprising one or both parents of the embryo;
creating a set of at least one ploidy state hypothesis for the at least one chromosome of the embryo, wherein each of the ploidy state hypothesis is one possible ploidy state of the at least one chromosome where 0, 1, or 2 copies of the chromosome come from each parent;
using two or more expert techniques which are algorithms operating on the measured genetic data to determine, for each expert technique used, a statistical probability of each ploidy state hypothesis in the set, given the measured genetic data, wherein the expert techniques are selected from:
a presence of homologs technique, which technique uses genetic data measured for both parents where one parent is heterozygous at a SNP and the other parent is homozygous at that SNP, wherein the presence of homologs technique comprises;
(1) phasing the measured genetic data from the parents and calculating noise floors per chromosome;
(2) segmenting the at least one chromosome;
(3) calculating SNP dropout rates per segment for parental genotypes of interest;
(4) calculating SNP dropout rates for each parent on the at least one chromosome and hypothesis likelihoods on each segment;
(5) combining the likelihoods across chromosome segments to produce a probability of data given parent strand hypothesis for whole chromosomes; and
(6) checking for invalid calls and calculating a probability for each ploidy state hypothesis;
a permutation technique, which technique compares the relationship between distributions of the measured genetic data of the embryo for different parental genotypes using a statistical algorithm to determine the probability of each ploidy state hypothesis given the measured genetic data; and
a presence of parent technique, which technique detects, independently for each parent, for a given chromosome of the at least one chromosome, whether or not there is a contribution from that parent's genome based on distances between sets of parental genotypes at the widest point on cumulative distribution function curves which plot observed distributions of measured genetic data for different parental genotypes, and assigns probabilities to each ploidy state hypothesis by calculating a summary statistic for each parent and comparing to data models for cases where a parent chromosome is present and cases where a parent chromosome is not present;
combining, for each ploidy state hypothesis, the statistical probabilities as determined by the two or more expert techniques to determine combined statistical probabilities;
determining the ploidy state for the at least one chromosome in the embryo based on the combined statistical probabilities of each of the ploidy state hypothesis, wherein the ploidy state with the highest combined statistical probability is determined to be the ploidy state of the at least one chromosome; and
using the determined ploidy state of the at least one chromosome to identify the embryo with a highest probability for developing into a euploid individual, from the set of embryos.

2. The method according to of claim 1, further comprising selecting at least one embryo from the set of embryos to transfer into a uterus, where the embryo(s) with a relatively higher likelihood of developing into a euploid individual is selected.

3. The method of claim 2, further comprising inserting the selected embryo(s) into a uterus.

4. The method of claim 1, wherein the method is capable of detecting a ploidy state from any of the ploidy states selected from euploidy, monosomy, uniparental disomy, matched trisomy, unmatched trisomy, and tetrasomy.

5. The method of claim 1, wherein the measured genetic data comprises single nucleotide polymorphism alleles measured using a genotyping array, DNA sequence data, and combinations thereof.

6. The method of claim 1, wherein the related individuals comprise both parents of the embryo.

7. The method of claim 1, wherein the method further comprises phasing the genetic data of one or both parents.

8. The method of claim 7, wherein the phasing is performed using an informatics based method.

9. The method of claim 8, further comprising determining phased genetic data of the embryo using an informatics based method.

10. The method of claim 1, wherein one of the selected expert techniques used is the presence of parents technique.

11. The method of claim 1, wherein one of the selected expert techniques used is the presence of homologs technique.

12. The method of claim 1, wherein one of the selected expert techniques is the permutations technique.

13. The method of claim 1, wherein the presence of parents technique, the presence of homologs technique, and the permutations technique are used to determine the ploidy state of the at least one chromosome.

14. The method of claim 1, wherein the genetic data is measured using a technique selected from the group consisting of molecular inversion probes, genotyping microarrays, a genotyping assay, fluorescence in-situ hybridization (FISH), sequencing, other high throughput genotyping platforms, and combinations thereof.

15. The method of claim 14, and wherein the genetic data is the measured responses at various single nucleotide polymorphism (SNP) loci on the at least one chromosomes.

16. The method of claim 15, wherein the method is capable of detecting a ploidy state from any of the ploidy states selected from euploidy, monosomy, uniparental disomy, matched trisomy, unmatched trisomy, and tetrasomy.

17. The method of claim 15, wherein the measured genetic data measured using a genotyping array, DNA sequence data, and combinations thereof.

18. The method of claim 1, further comprising using a statistical method to remove the bias in the genetic data before it is operated on by the two or more expert techniques.

19. The method of claim 1, wherein in addition to the two or more expert techniques, a whole chromosome mean technique is used to determine the ploidy state for the at least one chromosome, wherein the whole chromosome mean technique relies on the overall intensity of the measured genetic data, wherein a mean is determined for the measured internisities for certain sets of SNPs, and the characteristic behavior of the mean is used to determine the ploidy state of the at least one chromosome.

20. The method of claim 19, wherein the genetic data is normalized for variation in amplification before the characteristic behavior of the mean is used to determine the ploidy state of the at least one chromosome.
